SEQUENCE LISTING (120 sequences)

SEQ ID NO: 1 - protein, 317 aa, Halomonas elongata
MNPNYLDFEQPIAELQAKIEELRMVGNDSQVNLSDEIGRLEEKSRKLTESIFKDLSAWQVSQLSRHPQRPYTLDYLEHVFTDFDELHGDRRFADDAAIVGGVARLDDKPVMVIGHQKGRDVHEKVRRNFGMPRPEGYRKACRLMEMAERFHMPVLTFIDTPGAYPGIDAEERGQSEAIAYNLGVMSRLKTPIISTVVGEGGSGGALAIGVCDELAMLQYSTYSVISPEGCASILWKSADKASEAAQAMGITAERLKELGFVDTLIPEPLGGAHRQPSATAERIKTALLESLDRLETMETDALLERRYERLMSYGAPV

SEQ ID NO: 2 - DNA, 954 bp, Artificial Sequence
  Description of Artificial Sequence: Synthetic polynucleotide
atgaatccgaactatctggactttgaacaaccgatcgctgaactgcaagccaaaatcgaagaactgcgtatggtgggcaacgactcacaggtgaacctgtctgatgaaattggccgtctggaagaaaaaagtcgcaaactgaccgaatccatctttaaagacctgtcagcgtggcaagttagccaactgtctcgtcatccgcaacgcccgtataccctggattacctggaacatgtctttacggatttcgacgaactgcacggtgaccgtcgctttgcagatgacgcggccattgttggcggtgtcgctcgtctggatgacaaaccggtcatggtgatcggccatcagaaaggtcgtgatgtgcacgaaaaagttcgtcgcaacttcggcatgccgcgcccggaaggttatcgtaaagcgtgccgcctgatggaaatggccgaacgctttcacatgccggtgctgaccttcattgatacgccgggcgcatatccgggtatcgacgctgaagaacgtggccaaagcgaagcgattgcctacaatctgggtgttatgtcgcgcctgaaaaccccgattatcagcacggtggttggcgaaggcggttctggcggtgcactggctatcggtgtctgcgatgaactggcgatgctgcaatatagtacctactccgtgatttcaccggaaggctgtgcctcgatcctgtggaaaagcgcagataaagcttctgaagcagctcaagcgatgggcattaccgccgaacgtctgaaagaactgggtttcgttgacacgctgatcccggaaccgctgggcggtgcacatcgtcagccgagtgcgaccgccgaacgcattaaaacggccctgctggaaagcctggatcgcctggaaacgatggaaacggatgccctgctggaacgccgctatgaacgcctgatgtcttacggtgccccggtctga

SEQ ID NO: 3 - protein, 155 aa, Halomonas elongata
MDIRKVKKLIELLEESNISEIEIQEGEESVRISRHPNGTEHPQPAAPAWPATAAAPAPQPAAAPVESPAEVDEGPAYQGQPIVSPMVGTFYRAPAPGAKAFVELGQSVKKGETVCIVEAMKMMNQIEADRDGVVEAILVEDGEPVEFEQPMVVIS

SEQ ID NO: 4 - DNA, 468 bp, Artificial Sequence
  Description of Artificial Sequence: Synthetic polynucleotide
atggacatccgcaaagtgaaaaaactgatcgaactgctggaagaaagtaacatctctgaaattgaaatccaagaaggcgaagaaagcgtgcgtattagtcgccatccgaacggcaccgaacacccgcagccggcggcaccggcatggccggccacggcagctgcgccggcgccgcaaccggccgcagctccggtggaaagcccggcagaagttgatgaaggcccggcttatcagggtcaaccgatcgtttctccgatggtcggcaccttttaccgtgcgccggcaccgggtgcaaaagctttcgtcgaactgggccagagcgttaaaaaaggtgaaacggtctgcattgtggaagccatgaaaatgatgaatcaaatcgaagccgatcgcgacggtgtggttgaagcaatcctggtggaagatggtgaaccggtggaatttgaacagccgatggtggtgattagttaa

SEQ ID NO: 5 - protein, 446 aa, Halomonas elongata
MLDKVLIANRGEIALRILRACKELGIRTVAVHSKADRELMHVRLADEAVCIGPASSAQSYLNIPALISAAEVTDTSAIHPGYGFLSENADFAEQVERSGFTFIGPSAETIRLMGDKVSAINAMKEAGVPTVPGSNGPLGDDEGEILATARRIGYPVIIKAAAGGGGRGMRVVHAEGHLLSAVNVTRTEAHSSFGDGTVYMEKFLENPRHVEVQVLADGQGNAIHLYDRDCSLQRRHQKVLEEAPAPGLDQQAREQVFKACRDACVKIGYRGAGTFEFLYENGEFFFIEMNTRVQVEHPVTEMVTGVDIVREQLRIASGLPLSIRQEDVELSGHAFECRINAEDSRTFMPSPGRVTLYHPPGGLGVRMDSHVYTGYTVPPHYDSLIGKLITWGDDRETALIRMRNALDELLVEGIKTNTDLHKDLVRDGYFQQGGVNIHYLEKKLGL

SEQ ID NO: 6 - DNA, 1341 bp, Artificial Sequence
  Description of Artificial Sequence: Synthetic polynucleotide
atgctggacaaagtgctgattgcgaatcgtggcgaaattgcgctgcgtatcctgcgtgcgtgtaaagaactgggtatccgtaccgtcgctgttcattcaaaagcggaccgtgaactgatgcacgtccgcctggccgatgaagcagtgtgcattggtccggctagctctgcgcagtcgtatctgaacattccggcactgatcagtgcggccgaagtgaccgacacgtccgcgatccatccgggctacggtttcctgagcgaaaatgccgattttgcagaacaagtcgaacgttcaggtttcacctttattggcccgtcggccgaaacgatccgcctgatgggtgataaagttagtgctattaacgcgatgaaagaagcaggcgtgccgaccgttccgggttccaatggtccgctgggtgatgacgaaggtgaaattctggccaccgcacgtcgcatcggctatccggttattatcaaagcagctgcgggcggtggcggtcgtggtatgcgtgtggttcatgctgaaggccacctgctgagcgcggtcaacgtgacccgtacggaagcgcatagttccttcggcgatggcaccgtttatatggaaaaatttctggaaaacccgcgtcacgttgaagtccaggtgctggccgatggccagggtaatgcaattcatctgtacgatcgcgactgctctctgcaacgtcgccaccaaaaagtgctggaagaagctccggcaccgggtctggaccagcaagcacgtgaacaggtttttaaagcctgccgcgatgcatgtgtcaaaattggttatcgtggcgcgggcaccttcgaatttctgtacgaaaacggcgaatttttctttatcgaaatgaatacgcgcgttcaggtcgaacatccggtgaccgaaatggtcacgggtgtggatattgttcgtgaacagctgcgtatcgcatcaggtctgccgctgtcgattcgccaagaagacgttgaactgagcggtcatgccttcgaatgtcgtatcaatgcagaagatagccgcacctttatgccgtctccgggtcgtgtcacgctgtatcacccgccgggcggtctgggtgtccgtatggacagccatgtgtataccggctacacggttccgccgcactacgattctctgattggtaaactgatcacctggggcgatgaccgtgaaacggctctgattcgtatgcgcaacgccctggatgaactgctggttgaaggcatcaaaaccaatacggatctgcacaaagacctggttcgcgatggctactttcagcaaggcggtgtcaacattcactacctggaaaaaaaactgggtctgtaa

SEQ ID NO: 7 - protein, 346 aa, Halomonas elongata
MSWLDKIVPSVGRIQRKERRTSVPDGLWRKCPKCESVLYLPELEKHHNVCPKCDHHLRLTARKRLDWFLDKEGREEIAADLEPVDRLKFRDSKKYKDRLSAAQKATGEKDGLVAMRGTLEGLPVVAVAFEFTFMGGSMGAVVGEKFVRAATQALDEGVPLVCFSASGGARMQEALFSLMQMAKTSAALEKLKQAGVPYISVLTDPVFGGVSASLAMLGDLNIAEPNALIGFAGPRVIEQTVREQLPEGFQRSEFLLEHGAVDMIVHRQQIRERLGGVLRKLTHQPASGPAVVENDEPDLVDAAEQAEPQPEAPEAVETSESEAPTEKGVEADSEETDESPRSGDNR

SEQ ID NO: 8 - DNA, 1041 bp, Artificial Sequence
  Description of Artificial Sequence: Synthetic polynucleotide
atgtcttggttagataaaatcgtcccgtcagtgggtcgcattcaacgcaaagaacgtcgcacctcggtcccggatggtctgtggcgtaaatgcccgaaatgtgaatcagttctgtatctgccggaactggaaaaacatcacaacgtctgcccgaaatgtgatcatcacctgcgtctgaccgcgcgtaaacgcctggactggttcctggataaagaaggccgcgaagaaattgcggccgacctggaaccggtggatcgtctgaaatttcgcgacagcaaaaaatacaaagatcgtctgagcgcggcgcagaaagcaaccggtgaaaaagacggtctggtggccatgcgtggcacgctggaaggtctgccggtggttgcagttgcttttgaatttacctttatgggcggtagcatgggcgcagtcgtgggtgaaaaattcgttcgtgcggccacgcaggctctggatgaaggtgtgccgctggtttgcttcagcgcatctggcggtgcccgcatgcaggaagcactgtttagtctgatgcaaatggctaaaacctccgcagctctggaaaaactgaaacaggcgggcgtgccgtatatttctgttctgacggacccggtcttcggcggtgtgagtgcgtccctggccatgctgggtgatctgaacattgcagaaccgaatgctctgatcggctttgcgggtccgcgtgtcatcgaacagaccgtgcgcgaacaactgccggaaggcttccagcgttcagaatttctgctggaacatggtgccgttgatatgattgtccaccgtcagcaaatccgtgaacgcctgggcggtgtgctgcgcaaactgacgcatcaaccggcatcgggtccggccgttgtcgaaaatgatgaaccggacctggtcgatgcggccgaacaggcagaaccgcaaccggaagcaccggaagctgttgaaacctcagaatcggaagcaccgacggaaaaaggcgtggaagcagactcggaagaaacggatgaatcaccgcgctcaggcgacaaccgctaa

SEQ ID NO: 9 - protein, 604 aa, Artificial Sequence
  Description of Artificial Sequence: Synthetic polypeptide
MLDKIVIANRGEIALRILRACKELGIKTVAVHSSADRDLKHVLLADETVCIGPAPSVKSYLNIPAIISAAEITGAVAIHPGYGFLSENANFAEQVERSGFIFIGPKAETIRLMGDKVSAIAAMKKAGVPCVPGSDGPLGDDMDKNRAIAKRIGYPVIIKASGGGGGRGMRVVRGDAELAQSISMTRAEAKAAFSNDMVYMEKYLENPRHVEIQVLADGQGNAIYLAERDCSMQRRHQKVVEEAPAPGITPELRRYIGERCAKACVDIGYRGAGTFEFLFENGEFYFIEMNTRIQVEHPVTEMITGVDLIKEQLRIAAGQPLSIKQEEVHVRGHAVECRINAEDPNTFLPSPGKITRFHAPGGFGVRWESHIYAGYTVPPYYDSMIGKLICYGENRDVAIARMKNALQELIIDGIKTNVDLQIRIMNDENFQHGGTNIHYLEKKLGLQEKDIRKIKKLIELVEESGISELEISEGEESVRISRAAPAASFPVMQQAYAAPMMQQPAQSNAAAPATVPSMEAPAAAEISGHIVRSPMVGTFYRTPSPDAKAFIEVGQKVNVGDTLCIVEAMKMMNQIEADKSGTVKAILVESGQPVEFDEPLVVIE

SEQ ID NO: 10 - DNA, 1815 bp, Artificial Sequence
  Description of Artificial Sequence: Synthetic polynucleotide
atgctggacaagatcgtcatcgccaaccgcggcgaaatcgccctgcgcatcttgcgcgcgtgtaaagagctgggcattaagactgttgccgtgcattccagcgcagaccgcgacctgaagcatgttctgctggccgacgaaacggtttgcatcggtccggcaccgagcgtgaaaagctatctgaacatcccggccatcatctctgcggcagagatcaccggtgcagtggcgattcatccgggctacggtttcctgagcgagaacgctaactttgctgaacaagtggagcgtagcggtttcatcttcattggccctaaggcggagacgattcgcctgatgggcgacaaagtgagcgccattgcagcgatgaaaaaggccggtgtgccgtgtgttccgggcagcgatggtccgctgggtgacgatatggacaagaaccgtgccatcgctaaacgtattggctacccggtcattatcaaagcctctggtggtggcggtggccgtggtatgcgtgtcgtccgtggtgatgcggaactggcgcaaagcatcagcatgacccgtgcggaagccaaagcggcgttctctaacgatatggtgtatatggagaagtatctggagaatccgcgccacgttgaaatccaagttctggcggatggtcagggcaatgcgatctacttggcagaacgtgattgctccatgcaacgccgtcatcagaaggtggtggaagaggcaccggctccgggtattacgccggaactgcgtcgctacatcggtgagcgctgtgcgaaagcgtgtgtggacattggttaccgtggtgcgggtacgtttgagttcctgttcgaaaatggtgagttttacttcattgaaatgaatacccgcatccaggttgagcacccggtgaccgagatgattactggcgttgatctgatcaaagagcaactgcgcattgcggctggtcagccgctgtcgatcaagcaagaagaggtgcacgttcgtggtcacgcggtcgagtgccgtatcaatgcggaggacccgaatacctttctgccgagccctggtaagatcactcgttttcacgcgccaggtggttttggcgttcgttgggagtctcacatctacgccggttacaccgtgccgccgtactatgacagcatgattggtaaactgatctgctatggcgaaaatcgtgatgtcgcgatcgcccgcatgaaaaacgcgctgcaagagctgatcattgatggcattaagaccaatgtggatttgcagatccgcattatgaacgacgagaatttccagcacggcggtacgaacattcactacctggaaaagaaactgggcctgcaagagaaagacatccgcaagatcaagaagctgatcgaactggtggaagagtctggcatcagcgagctggagatcagcgaaggtgaagagagcgtccgtatttcccgtgcggcaccggcagcgagctttccggttatgcagcaagcatacgccgctccgatgatgcaacagccggcacagagcaacgccgctgcaccggcgaccgttccaagcatggaggcaccggcagcggccgagatttcgggtcatatcgtgcgtagcccgatggtgggcaccttctatcgcacgccgtcgccggacgcaaaagccttcatcgaagtcggccagaaggtcaatgtcggcgacacgctgtgtatcgttgaggcaatgaaaatgatgaaccagattgaagcggataagagcggtactgttaaagcgatcctggtggaatccggccagcctgttgagttcgatgaaccgctggttgtgatcgagtaa

SEQ ID NO: 11 - protein, 647 aa, Artificial Sequence
  Description of Artificial Sequence: Synthetic polypeptide
MSWIERIKSNITPTRKASIPEGVWTKCDSCGQVLYRAELERNLEVCPKCDHHMRMTARNRLHSLLDEGSLVELGSELEPKDVLKFRDSKKYKDRLASAQKETGEKDALVVMKGTLYGMPVVAAAFEFAFMGGSMGSVVGARFVRAVEQALEDNCPLICFSASGGARMQEALMSLMQMAKTSAALAKMQERGLPYISVLTDPTMGGVSASFAMLGDLNIAEPKALIGFAGPRVIEQTVREKLPPGFQRSEFLIEKGAIDMIVRRPEMRLKLASILAKLMNLPAPNPEAPREGVVVPPVPDQEPEALSGGGGSGGGGSGGGGSGGGGSAAASLNFLDFEQPIAELEAKIDSLTAVSRQDEKLDINIDEEVHRLREKSVELTRKIFADLGAWQIAQLARHPQRPYTLDYVRLAFDEFDELAGDRAYADDKAIVGGIARLDGRPVMIIGHQKGRETKEKIRRNFGMPAPEGYRKALRLMQMAERFKMPIITFIDTPGAYPGVGAEERGQSEAIARNLREMSRLGVPVVCTVIGEGGSGGALAIGVGDKVNMLQYSTYSVISPEGCASILWKSADKAPLAAEAMGIIAPRLKELKLIDSIIPEPLGGAHRNPEAMAASLKAQLLADLADLDVLSTEDLKNRRYQRLMSYGYA

SEQ ID NO: 12 - DNA, 1944 bp, Artificial Sequence
  Description of Artificial Sequence: Synthetic polynucleotide
atgagctggatcgagcgcatcaagagcaacatcaccccgacccgcaaggcgagcatccctgaaggcgtctggaccaaatgcgatagctgcggtcaggttttgtatcgtgcggagctggagcgtaacctggaagtgtgcccgaaatgcgaccatcacatgcgtatgaccgctcgtaatcgtctgcatagcctgctggatgagggcagcctggtcgagctgggtagcgaactggaaccgaaagatgttctgaaattccgtgattccaagaagtataaggatcgtttggcatctgcacaaaaagaaaccggtgagaaggacgcactggttgttatgaaaggcaccctgtatggtatgccggttgttgctgcggcgttcgagtttgcgtttatgggtggcagcatgggttccgtggtgggcgcacgctttgtgcgtgccgtggagcaggcgctggaggataactgtcctctgatttgtttcagcgcgagcggtggtgcgcgtatgcaagaggccctgatgagcctgatgcagatggcaaaaacctcggcagccctggcgaagatgcaagaacgcggcctgccgtacatttccgtcctgaccgaccctacgatgggcggtgtcagcgccagctttgcgatgctgggtgatttgaacatcgcagagccgaaggctctgattggttttgctggtccgcgtgttattgaacagacggttcgcgaaaagttgccgcctggtttccagcgcagcgagttcctgattgagaaaggtgccatcgacatgatcgttcgccgtccagaaatgcgtctgaaactggcgagcattctggcgaaattgatgaatctgccggctccgaatcctgaagcaccgcgtgagggtgtcgtggttccgccggtcccggaccaagagccggaggctctgagcggcggaggtggctctggtggaggcggttcaggaggcggtggcagtggtggcggcggatctgcggcagcttctctgaacttcctggacttcgagcagccgatcgccgaactggaggcgaagattgacagcctgaccgcggttagccgtcaagatgagaaactggacattaacatcgacgaagaggtccaccgtttgcgtgagaagtctgttgaactgactcgcaaaatctttgctgatttgggcgcatggcagattgcccagttggctcgccacccacaacgcccatataccctggactacgtgcgcctggcgtttgacgagttcgacgaactggcaggcgaccgcgcctatgcggacgataaagcaattgtcggcggtattgctcgtttggatggccgtccggtgatgattatcggccatcaaaaaggccgcgagacaaaggaaaagattcgtcgtaatttcggaatgccggcaccggagggctaccgcaaggccctgcgtctgatgcaaatggccgaacgctttaagatgccgattatcacgttcattgatacgcctggtgcgtacccaggcgttggtgcggaagagcgtggtcagagcgaggccatcgcacgtaacctgcgtgagatgtctcgtctgggtgtgccggtcgtttgcaccgtgattggcgagggcggtagcggtggtgcgttggcgatcggtgtcggtgataaggtcaacatgctgcaatacagcacgtacagcgtcattagcccggaaggttgcgcttccattctgtggaagagcgcggataaagcaccattggcagcggaagcgatgggtatcatcgcaccgcgtctgaaagaactgaagttgattgattctatcatcccggaaccgctgggcggtgctcaccgtaatccggaggcgatggcagccagcctgaaggcccagctgctggcggacctggcggatctggacgtgctgagcacggaggatctgaaaaaccgtcgctatcagcgcttgatgagctatggctacgcttaa

SEQ ID NO: 13 - protein, 566 aa, Synechococcus sp.
MQITNKIHFRNIRGDIFGGLTAAVIALPMALAFGVASGAGAEAGLWGAVLVGFFAALFGGTPTLISEPTGPMTVVMTAVIAHFTASAATPEEGLAIAFTVVMMAGVFQIIFGSLKLGKYVTMMPYTVISGFMSGIGIILVILQLAPFLGQASPGGGVIGTLQNLPTLLSNIQPGETALALGTVAIIWFMPEKFKKVIPPQLVALVLGTVIAFFVFPPEVSDLRRIGEIRAGFPELVRPSFSPVEFQRMILDAAVLGMLGCIDALLTSVVADSLTRTEHNSNKELIGQGLGNLFSGLFGGIAGAGATMGTVVNIQSGGRTALSGLVRAFVLLVVILGAASLTATIPLAVLAGIAFKVGVDIIDWSFLKRAHEISPKGALIMYGVILLTVLVDLIVAVGVGVFVANVLTIERMSNLQSEKVQTVSDADDNIRLTTTEKRWLDEGQGRVLLFQLSGPMIFGVAKAIAREHNAMGDCDALVFDIGEVPHMGVTASLALENAIEEALDKERQVYIVGAAGQTRRRLEKLKLFKRVPPDKCLMSREEALKNAVLGIYPHLADGVTAPSSEMG

SEQ ID NO: 14 - DNA, 1701 bp, Artificial Sequence
  Description of Artificial Sequence: Synthetic polynucleotide
atgcagattaccaacaagatccatttccgtaacattcgtggcgacatttttggtggcctgaccgctgctgtgattgcgctgccgatggcactggcttttggtgtggcaagtggtgcaggtgcagaagcaggtctgtggggtgcagttctggtgggctttttcgcagcactgttcggtggtacgccgaccctgatttcagaaccgacgggcccgatgaccgtggttatgacggccgtgatcgcacattttaccgcatcggcagctacgccggaagaaggcctggctattgcgttcaccgtcgtgatgatggccggtgtttttcagattatcttcggcagcctgaaactgggcaagtatgttaccatgatgccgtacacggtcatcagtggttttatgtccggtattggcattatcctggtgatcctgcagctggcaccgttcctgggtcaagccagtccgggcggtggcgttattggcaccctgcagaacctgccgacgctgctgtccaatatccaaccgggtgaaaccgccctggcactgggtacggtcgcgattatctggttcatgccggaaaagttcaagaaggttatcccgccgcagctggttgcgctggttctgggcaccgtcatcgcgtttttcgtgtttccgccggaagttagcgatctgcgtcgcattggcgaaatccgtgcaggtttcccggaactggtgcgtccgagcttttctccggttgaatttcagcgcatgattctggatgcggccgtgctgggcatgctgggttgcatcgatgcgctgctgaccagcgttgtcgccgactctctgacgcgtaccgaacataacagcaataaagaactgattggtcagggcctgggtaacctgttttctggcctgttcggtggtattgctggtgcaggtgcaacgatgggcaccgtggttaatatccaaagtggtggccgtaccgcactgtccggtctggtgcgtgcttttgttctgctggtcgtgattctgggtgcagcttctctgacggcaaccattccgctggctgtgctggcaggcatcgcctttaaagtgggtgttgatattatcgactggtcattcctgaaacgcgcccacgaaatctcgccgaagggcgcactgattatgtatggtgtgatcctgctgaccgtcctggtggatctgattgttgcggtcggcgtgggtgtttttgtcgccaacgttctgaccatcgaacgtatgtcaaatctgcagtcggaaaaagtccaaaccgtgagcgatgcggatgacaacattcgcctgaccacgaccgaaaagcgttggctggacgagggtcagggtcgtgtgctgctgtttcaactgtctggcccgatgattttcggtgttgcaaaagctatcgcgcgtgaacataacgcaatgggtgattgcgacgctctggtgtttgatattggcgaagtcccgcacatgggtgtgaccgcaagtctggctctggaaaatgcgattgaagaagccctggacaaagaacgccaggtttacatcgtcggtgcagcaggtcaaacccgtcgccgtctggaaaaactgaagctgtttaaacgcgtgccgccggataagtgtctgatgtcccgtgaagaagcactgaagaatgctgttctgggtatctatccgcatctggctgacggtgttacggctccgagttccgaaatgggctaa

SEQ ID NO: 15 - DNA, 1071 bp, Sulfolobus tokodaii
atgtctcgtcgcaccctgaaagcggctatcctgggcgccaccggcctggttggtatcgaatatgtccgtatgctgtcaaatcatccgtatatcaaaccggcgtatctggccggcaaaggttcagttggcaaaccgtacggtgaagtggttcgttggcagaccgttggccaagtcccgaaagaaatcgccgatatggaaattaaaccgacggacccgaaactgatggatgacgtggatattatcttttcgccgctgccgcagggtgcggccggtccggttgaagaacaatttgcaaaagaaggcttcccggtcatcagcaactctccggatcatcgtttcgatccggacgtcccgctgctggtgccggaactgaatccgcacaccattagtctgatcgatgaacagcgcaaacgtcgcgaatggaaaggttttattgttaccacgccgctgtgcacggcacaaggtgcagctatcccgctgggtgctatcttcaaagattacaaaatggacggcgcgttcattaccacgatccagagtctgtccggtgcaggttacccgggtatcccgtctctggatgtcgtggacaacattctgccgctgggcgatggttatgacgcgaaaaccattaaagaaatcttccgtattctgtcagaagttaaacgcaatgtcgatgaaccgaaactggaagacgtttcgctggcggccaccacgcatcgtatcgccaccattcatggccactatgaagtgctgtacgttagttttaaagaagaaaccgcagctgaaaaagtgaaagaaacgctggaaaacttccgcggtgaaccgcaggatctgaaactgccgaccgcaccgtccaaaccgattatcgtcatgaatgaagatacgcgtccgcaagtgtactttgatcgctgggctggcgacattccgggtatgagcgttgtcgtgggccgtctgaaacaggtgaacaaacgtatgatccgcctggtgtctctgattcacaataccgttcgcggtgcggcgggcggtggcatcctggctgctgaactgctggttgaaaaaggttacattgaaaaa

SEQ ID NO: 16 - protein, 357 aa, Sulfolobus tokodaii
MSRRTLKAAILGATGLVGIEYVRMLSNHPYIKPAYLAGKGSVGKPYGEVVRWQTVGQVPKEIADMEIKPTDPKLMDDVDIIFSPLPQGAAGPVEEQFAKEGFPVISNSPDHRFDPDVPLLVPELNPHTISLIDEQRKRREWKGFIVTTPLCTAQGAAIPLGAIFKDYKMDGAFITTIQSLSGAGYPGIPSLDVVDNILPLGDGYDAKTIKEIFRILSEVKRNVDEPKLEDVSLAATTHRIATIHGHYEVLYVSFKEETAAEKVKETLENFRGEPQDLKLPTAPSKPIIVMNEDTRPQVYFDRWAGDIPGMSVVVGRLKQVNKRMIRLVSLIHNTVRGAAGGGILAAELLVEKGYIEK

SEQ ID NO: 17 - DNA, 1098 bp, Escherichia coli
atgtcatctgaaaaactgtattccccactgaaagtgggcgcgatcacggcggcaaaccgtatttttatggcaccgctgacgcgtctgcgcagtattgaaccgggtgacattcctaccccgttgatggcggaatactatcgccaacgtgccagtgccggtttgattattagtgaagccacgcaaatttctgcccaggcaaaaggatatgcaggtgcgcctggcatccatagtccggagcaaattgccgcatggaaaaaaatcaccgctggcgttcatgctgaaaatggtcatatggccgtgcagctgtggcacaccggacgcatttctcacgccagcctgcaacctggcggtcaggcaccggtagcgccttcagcacttagcgcgggaacacgtacttctctgcgcgatgaaaatggtcaggcgatccgtgttgaaacatccatgccgcgtgcgcttgaactggaagagattccaggtatcgtcaatgatttccgtcaggccattgctaacgcgcgtgaagccggttttgatctggtagagctccactctgctcacggttatttgctgcatcagttcctttctccttcttcaaaccatcgtaccgatcagtacggcggcagcgtggaaaatcgcgcacgtttggtactggaagtggtcgatgccgggattgaagaatggggtgccgatcgcattggcattcgcgtttcaccaatcggtactttccagaacacagataacggcccgaatgaagaagccgatgcactgtatctgattgaacaactgggtaaacgcggcattgcttatctgcatatgtcagaaccagattgggcggggggtgaaccgtatactgatgcgttccgcgaaaaagtacgcgcccgtttccacggtccgattatcggcgcaggtgcatacacagtagaaaaagctgaaacgctgatcggcaaagggttaattgatgcggtggcatttggtcgtgactggattgcgaacccggatctggtcgcccgcttgcagcgcaaagctgagcttaacccacagcgtgccgaaagtttctacggtggcggcgcggaaggctataccgattacccgacgttgtaa

SEQ ID NO: 18 - protein, 365 aa, Escherichia coli
MSSEKLYSPLKVGAITAANRIFMAPLTRLRSIEPGDIPTPLMAEYYRQRASAGLIISEATQISAQAKGYAGAPGIHSPEQIAAWKKITAGVHAENGHMAVQLWHTGRISHASLQPGGQAPVAPSALSAGTRTSLRDENGQAIRVETSMPRALELEEIPGIVNDFRQAIANAREAGFDLVELHSAHGYLLHQFLSPSSNHRTDQYGGSVENRARLVLEVVDAGIEEWGADRIGIRVSPIGTFQNTDNGPNEEADALYLIEQLGKRGIAYLHMSEPDWAGGEPYTDAFREKVRARFHGPIIGAGAYTVEKAETLIGKGLIDAVAFGRDWIANPDLVARLQRKAELNPQRAESFYGGGAEGYTDYPTL

SEQ ID NO: 19 - DNA, 591 bp, Escherichia coli
atgaacgaagccgttagcccaggtgcgcttagcaccctgttcaccgatgcccgcactcacaacggctggcgggagacacccgtcagcgatgagacgttacgggagatttatgccctgatgaaatgggggccgacatcagctaactgttctccggcacggatcgtgtttacccgcacggcagaaggaaaagaacgtctgcgcccggcactttccagcggcaatctgcaaaaaaccctgaccgcgcccgtcaccgctatcgtcgcctgggacagtgaattttatgaacggttaccactactgtttccccacggtgatgcccgcagttggtttacctccagcccacaacttgccgaagaaacagcgtttcgcaacagttccatgcaggcggcctatctgatcgtcgcctgccgggcgctgggactggataccggcccgatgtcgggctttgaccgtcaacacgtggacgacgccttttttacgggcagcacgctgaagagcaatctgctgattaatatcggctatggcgatagcagcaagctttatgcgcgcctgccacgtctgtcctttgaagaagcctgcgggctgttgtaa

SEQ ID NO: 20 - protein, 196 aa, Escherichia coli
MNEAVSPGALSTLFTDARTHNGWRETPVSDETLREIYALMKWGPTSANCSPARIVFTRTAEGKERLRPALSSGNLQKTLTAPVTAIVAWDSEFYERLPLLFPHGDARSWFTSSPQLAEETAFRNSSMQAAYLIVACRALGLDTGPMSGFDRQHVDDAFFTGSTLKSNLLINIGYGDSSKLYARLPRLSFEEACGLL

SEQ ID NO: 21 - DNA, 747 bp, Escherichia coli
atggtcgttttagtaactggagcaacggcaggttttggtgaatgcattactcgtcgttttattcaacaagggcataaagttatcgccactggccgtcgccaggaacggttgcaggagttaaaagacgaactgggagataatctgtatatcgcccaactggacgttcgcaaccgcgccgctattgaagagatgctggcatcgcttcctgccgagtggtgcaatattgatatcctggtaaataatgccggcctggcgttgggcatggagcctgcgcataaagccagcgttgaagactgggaaacgatgattgataccaacaacaaaggcctggtatatatgacgcgcgccgtcttaccgggtatggttgaacgtaatcatggtcatattattaacattggctcaacggcaggtagctggccgtatgccggtggtaacgtttacggtgcgacgaaagcgtttgttcgtcagtttagcctgaatctgcgtacggatctgcatggtacggcggtgcgcgtcaccgacatcgaaccgggtctggtgggtggtaccgagttttccaatgtccgctttaaaggcgatgacggtaaagcagaaaaaacctatcaaaataccgttgcattgacgccagaagatgtcagcgaagccgtctggtgggtgtcaacgctgcctgctcacgtcaatatcaataccctggaaatgatgccggttacccaaagctatgccggactgaatgtccaccgtcagtaa

SEQ ID NO: 22 - protein, 248 aa, Escherichia coli
MVVLVTGATAGFGECITRRFIQQGHKVIATGRRQERLQELKDELGDNLYIAQLDVRNRAAIEEMLASLPAEWCNIDILVNNAGLALGMEPAHKASVEDWETMIDTNNKGLVYMTRAVLPGMVERNHGHIINIGSTAGSWPYAGGNVYGATKAFVRQFSLNLRTDLHGTAVRVTDIEPGLVGGTEFSNVRFKGDDGKAEKTYQNTVALTPEDVSEAVWWVSTLPAHVNINTLEMMPVTQSYAGLNVHRQ

SEQ ID NO: 23 - DNA, 897 bp, Pseudomonas aeruginosa
atggccgacattgcgtttctgggtctgggcaatatgggcggtccgatggccgcgaacctgctgaaagccggccaccgtgtgaatgtgttcgacctgcaaccaaaagcggtcctgggcttggttgagcaaggcgcgcagggcgcagactctgctctgcaatgttgtgagggtgcggaggtcgtgatttctatgctgccagcaggccagcatgtggaaagcctgtacctgggcgatgatggtctgctggcacgcgtggcgggcaagcctttgctgattgactgtagcaccatcgcaccggaaacggcgcgtaaggtggcggaggcagccgcagcaaagggcctgacgctgctggatgccccggtttcgggcggtgtcggtggtgcccgtgcaggtacgctgtcgtttatcgtgggtggtccggcggagggttttgcgcgtgcgcgtccggttctggagaatatgggtcgcaacattttccacgcgggtgatcacggcgctggtcaggtggcgaaaatctgtaacaacatgctgctgggtatcttgatggcgggcaccgccgaagccttggcgctgggcgtcaaaaacggtctggacccggcagtgctgtccgaagtgatgaaacagagcagcggtggtaactgggcgctgaatctgtacaatccgtggccgggtgtgatgccgcaggccccagcctctaatggctacgcaggcggcttccaagtgcgcctgatgaacaaagacctgggcctggcgctggcgaatgcgcaagcggtccaagcgagcaccccgctgggcgcactggcccgtaacctgtttagcctgcacgctcaagccgacgccgagcacgaaggtctggacttcagctctattcaaaaactgtatcgcggtaaggattag

SEQ ID NO: 24 - protein, 298 aa, Pseudomonas aeruginosa
MADIAFLGLGNMGGPMAANLLKAGHRVNVFDLQPKAVLGLVEQGAQGADSALQCCEGAEVVISMLPAGQHVESLYLGDDGLLARVAGKPLLIDCSTIAPETARKVAEAAAAKGLTLLDAPVSGGVGGARAGTLSFIVGGPAEGFARARPVLENMGRNIFHAGDHGAGQVAKICNNMLLGILMAGTAEALALGVKNGLDPAVLSEVMKQSSGGNWALNLYNPWPGVMPQAPASNGYAGGFQVRLMNKDLGLALANAQAVQASTPLGALARNLFSLHAQADAEHEGLDFSSIQKLYRGKD

SEQ ID NO: 25 - DNA, 900 bp, Pseudomonas aeruginosa
atgggcaaacagatcgccttcatcggcctgggccatatgggcgcacctatggccaccaacctgctgaaggccggctacctgctgaatgtgttcgacctggtgcagagcgccgtggatggtttagtggccgcaggtgcaagtgcagcacgcagtgcacgcgatgccgttcagggtgccgacgtggtgatcagcatgctgcctgccagccaacacgtggagggtctgtacctggacgacgatggtctgctggcccacattgcccctggcaccttagtgctggagtgcagcacaatcgccccgaccagtgcacgcaagattcatgcagcagcccgcgagcgtggtctggcaatgctggacgcaccggttagcggtggtacagcaggtgccgcagcaggcaccctgaccttcatggtgggcggtgacgccgaagccctggaaaaagcacgcccgctgtttgaggcaatgggccgtaacatcttccatgccggccctgatggcgcaggtcaggtggccaaagtgtgcaataaccagctgctggcagtgctgatgatcggtaccgccgaggcaatggcactgggcgtggcaaacggcttagaggccaaggtgctggcagaaatcatgcgccgtagtagcggcggtaactgggccctggaggtgtacaacccgtggcctggcgtgatggagaatgcaccggccagtcgtgactacagcggcggtttcatggcacagctgatggccaaggacctgggcttagcccaagaggcagcccaagccagcgccagtagtaccccgatgggcagcttagccctgagtctgtaccgcttactgctgaagcagggctacgccgaacgcgacttcagcgtggtgcagaagctgttcgacccgacccaaggccagtaa

SEQ ID NO: 26 - protein, 299 aa, Pseudomonas aeruginosa
MGKQIAFIGLGHMGAPMATNLLKAGYLLNVFDLVQSAVDGLVAAGASAARSARDAVQGADVVISMLPASQHVEGLYLDDDGLLAHIAPGTLVLECSTIAPTSARKIHAAARERGLAMLDAPVSGGTAGAAAGTLTFMVGGDAEALEKARPLFEAMGRNIFHAGPDGAGQVAKVCNNQLLAVLMIGTAEAMALGVANGLEAKVLAEIMRRSSGGNWALEVYNPWPGVMENAPASRDYSGGFMAQLMAKDLGLAQEAAQASASSTPMGSLALSLYRLLLKQGYAERDFSVVQKLFDPTQGQ

SEQ ID NO: 27 - protein, 262 aa, Escherichia coli
MGFLSGKRILVTGVASKLSIAYGIAQAMHREGAELAFTYQNDKLKGRVEEFAAQLGSDIVLQCDVAEDASIDTMFAELGKVWPKFDGFVHSIGFAPGDQLDGDYVNAVTREGFKIAHDISSYSFVAMAKACRSMLNPGSALLTLSYLGAERAIPNYNVMGLAKASLEANVRYMANAMGPEGVRVNAISAGPIRTLAASGIKDFRKMLAHCEAVTPIRRTVTIEDVGNSAAFLCSDLSAGIFGEVVHVDGGFSIAAMNELELK

SEQ ID NO: 28 - protein, 406 aa, Escherichia coli
MKRAVITGLGIVSSIGNNQQEVLASLREGRSGITFSQELKDSGMRSHVWGNVKLDTTGLIDRKVVRFMSDASIYAFLSMEQAIADAGLSPEAYQNNPRVGLIAGSGGGSPRFQVFGADAMRGPRGLKAVGPYVVTKAMASGVSACLATPFKIHGVNYSISSACATSAHCIGNAVEQIQLGKQDIVFAGGGEELCWEMACEFDAMGALSTKYNDTPEKASRTYDAHRDGFVIAGGGGMVVVEELEHALARGAHIYAEIVGYGATSDGADMVAPSGEGAVRCMKMAMHGVDTPIDYLNSHGTSTPVGDVKELAAIREVFGDKSPAISATKVMTGHSLGAAGVQEAIYSLLMLEHGFIAPSINIEELDEQAAGLNIVTETTDRELTTVMSNSFGFGGTNATLVMRKLKD

SEQ ID NO: 29 - protein, 309 aa, Escherichia coli
MTQFAFVFPGQGSQTVGMLADMAASYPIVEETFAEASAALGYDLWALTQQGPAEELNKTWQTQPALLTASVALYRVWQQQGGKAPAMMAGHSLGEYSALVCAGVIDFADAVRLVEMRGKFMQEAVPEGTGAMAAIIGLDDASIAKACEEAAEGQVVSPVNFNSPGQVVIAGHKEAVERAGAACKAAGAKRALPLPVSVPSHCALMKPAADKLAVELAKITFNAPTVPVVNNVDVKCETNGDAIRDALVRQLYNPVQQTKSVEYMAAQGVEHLYEVGPGKVLTGLTKRIVDTLTASALNEPSAMAAALEL

SEQ ID NO: 30 - DNA, 888 bp, Escherichia coli
atgagcggttcattacgtaaaatgccggtctggttaccaatagtcatattgctcgttgccatggcgtctattcagggtggagcctcgttagctaagtcactttttcctctggtgggcgcaccgggtgtcactgcgctgcgtctggcattaggaacgctgatcctcatcgcgttctttaagccatggcgactgcgctttgccaaagagcaacggttaccgctgttgttttacggcgtttcgctgggtgggatgaattatcttttttatctttctattcagacagtaccgctgggtattgcggtggcgctggagttcaccggaccactggcggtggcgctgttctcttctcgtcgcccggtagatttcgtctgggttgtgctggcggttcttggtctgtggttcctgctaccgctggggcaagacgtttcccatgtcgatttaaccggctgtgcgctggcactgggggccggggcttgttgggctatttacattttaagtgggcaacgcgcaggagcggaacatggccctgcgacggtggcaattggttcgttgattgcagcgttaattttcgtgccaattggagcgcttcaggctggtgaagcactctggcactggtcggttattccattgggtctggctgtcgctattctctcgaccgctctgccttattcgctggaaatgattgccctcacccgtttgccaacacggacatttggtacgctgatgagcatggaaccggcgctggctgccgtttccgggatgattttcctcggagaaacactgacacccatacagctactggcgctcggcgctatcatcgccgcttcaatggggtctacgctgacagtacgcaaagagagcaaaataaaagaattagacattaattaa

SEQ ID NO: 31 - protein, 295 aa, Escherichia coli
MSGSLRKMPVWLPIVILLVAMASIQGGASLAKSLFPLVGAPGVTALRLALGTLILIAFFKPWRLRFAKEQRLPLLFYGVSLGGMNYLFYLSIQTVPLGIAVALEFTGPLAVALFSSRRPVDFVWVVLAVLGLWFLLPLGQDVSHVDLTGCALALGAGACWAIYILSGQRAGAEHGPATVAIGSLIAALIFVPIGALQAGEALWHWSVIPLGLAVAILSTALPYSLEMIALTRLPTRTFGTLMSMEPALAAVSGMIFLGETLTPIQLLALGAIIAASMGSTLTVRKESKIKELDIN

SEQ ID NO: 32 - DNA, 1269 bp, Escherichia coli
atgcgactcctccggcaaaacggaagtttatcacttgtgcgttataacggacaaatgctacggtgcctgtacgctataacgcacgaggtgactatgcgtctgttttctattcctccacccacgctactggcggggtttctggcggtattaattggctacgccagttcagcggcaataatctggcaagcagcgattgtcgccggagccaccactgcacaaatctctggctggatgacggcgctggggctggcaatgggcgtcagtacgctgactctgacattatggtatcgcgtacctgttctcaccgcatggtcaacgcctggcgcggctttgttggtcaccggattgcagggactaacacttaacgaagccatcggcgtttttattgtcaccaacgcgctaatagtcctctgcggcataacgggactctttgctcgtctgatgcgcattattccgcactcgcttgcggcggcaatgcttgccgggattttattacgctttggtttacaggcgtttgccagtctggacggtcaatttacgttgtgtggaagtatgttgctggtatggctggcaaccaaggccgttgcgccgcgctatgcggtaattgccgcgatgattattgggatcgtgatcgtcatcgcgcaaggtgacgttgtcacaactgatgttgtctttaaacccgttctccccacttatattacccctgatttttcgtttgctcacagcctgagcgttgcactccccctttttctggtgacgatggcatcgcaaaacgcaccgggtatcgcagcaatgaaagcagctggatattcggctcctgtttcgccattaattgtatttactggattgctggcactggttttttcccctttcggcgtttattccgtcggtattgcggcaatcaccgcggctatttgccaaagcccggaagcgcatccggataaagatcaacgttggctggccgctgccgttgcaggcattttctatttgctcgcaggtctgtttggtagtgccattaccgggatgatggctgccctgcccgtaagttggatccagatgctggcaggtctggcgctgttaagtaccatcggcggcagtttgtatcaggcgctgcataatgagcgtgagcgagacgcggcggtggtggcatttctggtaacggcaagtggattgacgctggtcgggattggttctgcgttttggggattaattgccggaggcgtttgttacgtggtgttgaatttaatcgctgacagaaaccgatattga

SEQ ID NO: 33 - protein, 422 aa, Escherichia coli
MRLLRQNGSLSLVRYNGQMLRCLYAITHEVTMRLFSIPPPTLLAGFLAVLIGYASSAAIIWQAAIVAGATTAQISGWMTALGLAMGVSTLTLTLWYRVPVLTAWSTPGAALLVTGLQGLTLNEAIGVFIVTNALIVLCGITGLFARLMRIIPHSLAAAMLAGILLRFGLQAFASLDGQFTLCGSMLLVWLATKAVAPRYAVIAAMIIGIVIVIAQGDVVTTDVVFKPVLPTYITPDFSFAHSLSVALPLFLVTMASQNAPGIAAMKAAGYSAPVSPLIVFTGLLALVFSPFGVYSVGIAAITAAICQSPEAHPDKDQRWLAAAVAGIFYLLAGLFGSAITGMMAALPVSWIQMLAGLALLSTIGGSLYQALHNERERDAAVVAFLVTASGLTLVGIGSAFWGLIAGGVCYVVLNLIADRNRY

SEQ ID NO: 34 - protein, 1037 aa, Escherichia coli
MANFFIDRPIFAWVLAILLCLTGTLAIFSLPVEQYPDLAPPNVRVTANYPGASAQTLENTVTQVIEQNMTGLDNLMYMSSQSSGTGQASVTLSFKAGTDPDEAVQQVQNQLQSAMRKLPQAVQNQGVTVRKTGDTNILTIAFVSTDGSMDKQDIADYVASNIQDPLSRVNGVGDIDAYGSQYSMRIWLDPAKLNSFQMTAKDVTDAIESQNAQIAVGQLGGTPSVDKQALNATINAQSLLQTPEQFRDITLRVNQDGSEVRLGDVATVEMGAEKYDYLSRFNGKPASGLGVKLASGANEMATAELVLNRLDELAQYFPHGLEYKVAYETTSFVKASIEDVVKTLLEAIALVFLVMYLFLQNFRATLIPTIAVPVVLMGTFSVLYAFGYSVNTLTMFAMVLAIGLLVDDAIVVVENVERIMSEEGLTPREATRKSMGQIQGALVGIAMVLSAVFVPMAFFGGTTGAIYRQFSITIVAAMVLSVLVAMILTPALCATLLKPLKKGEHHGQKGFFAWFNQMFNRNAERYEKGVAKILHRSLRWIVIYVLLLGGMVFLFLRLPTSFLPLEDRGMFTTSVQLPSGSTQQQTLKVVEQIEKYYFTHEKDNIMSVFATVGSGPGGNGQNVARMFIRLKDWSERDSKTGTSFAIIERATKAFNQIKEARVIASSPPAISGLGSSAGFDMELQDHAGAGHDALMAARNQLLALAAENPELTRVRHNGLDDSPQLQIDIDQRKAQALGVAIDDINDTLQTAWGSSYVNDFMDRGRVKKVYVQAAAPYRMLPDDINLWYVRNKDGGMVPFSAFATSRWETGSPRLERYNGYSAVEIVGEAAPGVSTGTAMDIMESLVKQLPNGFGLEWTAMSYQERLSGAQAPALYAISLLVVFLCLAALYESWSVPFSVMLVVPLGVIGALLATWMRGLENDVYFQVGLLTVIGLSAKNAILIVEFANEMNQKGHDLFEATLHACRQRLRPILMTSLAFIFGVLPMATSTGAGSGGQHAVGTGVMGGMISATILAIYFVPLFFVLVRRRFPLKPRPE

SEQ ID NO: 35 - protein, 591 aa, Escherichia coli
MAHPPLLHLQDITLSLGGNPLLDGAGFAVGRGERLCLVGRNGSGKSTLLKIAAGVIQPDSGSVFVQPGASLRYLPQEPDLSAYATTADYVVGQIGDPDMAWRATPLLDALGLTGRESTQNLSGGEGRRCAIAGVLAAAPDVLLLDEPTNHLDMPTIEWLERELLSLGAMVIISHDRRLLSTLSRSVVWLDRGVTRRLDEGFGRFEAWREEVLEQEERDAHKLDRKIAREEDWMRYGVTARRKRNVRRVRELADLRTARKEAIRAPGTLTLNTQLRPHRKLVAVAEDISKAWGEKQVVRHLDLRILRGDRLGIVGANGAGKTTLLRMLTGLDQPDSGTISLGPSLNMVTLDQQRRTLNPERTLADTLTEGGGDMVQVGTEKRHVVGYMKDFLFRPEQARTPVSALSGGERGRLMLACALAKPSNLLVLDEPTNDLDLETLDILQDMLASCEGTVLLVSHDRDFLDRVATSVLATEGDGNWIEYAGGYSDMLAQRHQKPLTTASVVENEPTKPKETTAARGPTKKLSYKDQFALDNLPKEMEKLEAQAANCVKNWQIQIYMEKTPRSLRNFRLIYRSSKQSWQNLKNAGWNWK

SEQ ID NO: 36 - protein, 396 aa, Escherichia coli
MTTRQHSSFAIVFILGLLAMLMPLSIDMYLPALPVISAQFGVPAGSTQMTLSTYILGFALGQLIYGPMADSFGRKPVVLGGTLVFAAAAVACALANTIDQLIVMRFFHGLAAAAASVVINALMRDIYPKEEFSRMMSFVMLVTTIAPLMAPIVGGWVLVWLSWHYIFWILALAAILASAMIFFLIKETLPPERRQPFHIRTTIGNFAALFRHKRVLSYMLASGFSFAGMFSFLSAGPFVYIEINHVAPENFGYYFALNIVFLFVMTIFNSRFVRRIGALNMFRSGLWIQFIMAAWMVISALLGLGFWSLVVGVAAFVGCVSMVSSNAMAVILDEFPHMAGTASSLAGTFRFGIGAIVGALLSLATFNSAWPMIWSIAFCATSSILFCLYASRPKKR

SEQ ID NO: 37 - protein, 1047 aa, Escherichia coli
MIEWIIRRSVANRFLVLMGALFLSIWGTWTIINTPVDALPDLSDVQVIIKTSYPGQAPQIVENQVTYPLTTTMLSVPGAKTVRGFSQFGDSYVYVIFEDGTDPYWARSRVLEYLNQVQGKLPAGVSAELGPDATGVGWIYEYALVDRSGKHDLADLRSLQDWFLKYELKTIPDVAEVASVGGVVKEYQVVIDPQRLAQYGISLAEVKSALDASNQEAGGSSIELAEAEYMVRASGYLQTLDDFNHIVLKASENGVPVYLRDVAKVQIGPEMRRGIAELNGEGEVAGGVVILRSGKNAREVIAAVKDKLETLKSSLPEGVEIVTTYDRSQLIDRAIDNLSGKLLEEFIVVAVVCALFLWHVRSALVAIISLPLGLCIAFIVMHFQGLNANIMSLGGIAIAVGAMVDAAIVMIENAHKRLEEWQHQHPDATLDNKTRWQVITDASVEVGPALFISLLIITLSFIPIFTLEGQEGRLFGPLAFTKTYAMAGAALLAIVVIPILMGYWIRGKIPPESSNPLNRFLIRVYHPLLLKVLHWPKTTLLVAALSVLTVLWPLNKVGGEFLPQINEGDLLYMPSTLPGISAAEAASMLQKTDKLIMSVPEVARVFGKTGKAETATDSAPLEMVETTIQLKPQEQWRPGMTMDKIIEELDNTVRLPGLANLWVPPIRNRIDMLSTGIKSPIGIKVSGTVLADIDAMAEQIEEVARTVPGVASALAERLEGGRYINVEINREKAARYGMTVADVQLFVTSAVGGAMVGETVEGIARYPINLRYPQSWRDSPQALRQLPILTPMKQQITLADVADIKVSTGPSMLKTENARPTSWIYIDARDRDMVSVVHDLQKAIAEKVQLKPGTSVAFSGQFELLERANHKLKLMVPMTLMIIFVLLYLAFRRVGEALLIISSVPFALVGGIWLLWWMGFHLSVATGTGFIALAGVAAEFGVVMLMYLRHAIEAVPSLNNPQTFSEQKLDEALYHGAVLRVRPKAMTVAVIIAGLLPILWGTGAGSEVMSRIAAPMIGGMITAPLLSLFIIPAAYKLMWLHRHRVRK

SEQ ID NO: 38 - protein, 384 aa, Escherichia coli
MLLVLVLIGLNMRPLLTSVGPLLPQLRQASGMSFSVAALLTALPVVTMGGLALAGSWLHQHVSERRSVAISLLLIAVGALMRELYPQSALLLSSALLGGVGIGIIQAVMPSVIKRRFQQRTPLVMGLWSAALMGGGGLGAAITPWLVQHSETWYQTLAWWALPAVVALFAWWWQSAREVASSHKTTTTPVRVVFTPRAWTLGVYFGLINGGYASLIAWLPAFYIEIGASAQYSGSLLALMTLGQAAGALLMPAMARHQDRRKLLMLALVLQLVGFCGFIWLPMQLPVLWAMVCGLGLGGAFPLCLLLALDHSVQPAIAGKLVAFMQGIGFIIAGLAPWFSGVLRSISGNYLMDWAFHALCVVGLMIITLRFAPVRFPQLWVKEA

SEQ ID NO: 39 - protein, 219 aa, Escherichia coli
MDLIYFLIDFILHIDVHLAELVAEYGVWVYAILFLILFCETGLVVTPFLPGDSLLFVAGALASLETNDLNVHMMVVLMLIAAIVGDAVNYTIGRLFGEKLFSNPNSKIFRRSYLDKTHQFYEKHGGKTIILARFVPIVRTFAPFVAGMGHMSYRHFAAYNVIGALLWVLLFTYAGYFFGTIPMVQDNLKLLIVGIIVVSILPGVIEIIRHKRAAARAAK

SEQ ID NO: 40 - protein, 299 aa, Escherichia coli
MSRKDGVLALLVVVVWGLNFVVIKVGLHNMPPLMLAGLRFMLVAFPAIFFVARPKVPLNLLLGYGLTISFAQFAFLFCAINFGMPAGLASLVLQAQAFFTIMLGAFTFGERLHGKQLAGIALAIFGVLVLIEDSLNGQHVAMLGFMLTLAAAFSWACGNIFNKKIMSHSTRPAVMSLVIWSALIPIIPFFVASLILDGSATMIHSLVTIDMTTILSLMYLAFVATIVGYGIWGTLLGRYETWRVAPLSLLVPVVGLASAALLLDERLTGLQFLGAVLIMTGLYINVFGLRWRKAVKVGS

SEQ ID NO: 41 - protein, 195 aa, Escherichia coli
MTPTLLSAFWTYTLITAMTPGPNNILALSSATSHGFRQSTRVLAGMSLGFLIVMLLCAGISFSLAVIDPAAVHLLSWAGAAYIVWLAWKIATSPTKEDGLQAKPISFWASFALQFVNVKIILYGVTALSTFVLPQTQALSWVVGVSVLLAMIGTFGNVCWALAGHLFQRLFRQYGRQLNIVLALLLVYCAVRIFY

SEQ ID NO: 42 - protein, 512 aa, Escherichia coli
MQQQKPLEGAQLVIMTIALSLATFMQVLDSTIANVAIPTIAGNLGSSLSQGTWVITSFGVANAISIPLTGWLAKRVGEVKLFLWSTIAFAIASWACGVSSSLNMLIFFRVIQGIVAGPLIPLSQSLLLNNYPPAKRSIALALWSMTVIVAPICGPILGGYISDNYHWGWIFFINVPIGVAVVLMTLQTLRGRETRTERRRIDAVGLALLVIGIGSLQIMLDRGKELDWFSSQEIIILTVVAVVAICFLIVWELTDDNPIVDLSLFKSRNFTIGCLCISLAYMLYFGAIVLLPQLLQEVYGYTATWAGLASAPVGIIPVILSPIIGRFAHKLDMRRLVTFSFIMYAVCFYWRAYTFEPGMDFGASAWPQFIQGFAVACFFMPLTTITLSGLPPERLAAASSLSNFTRTLAGSIGTSITTTMWTNRESMHHAQLTESVNPFNPNAQAMYSQLEGLGMTQQQASGWIAQQITNQGLIISANEIFWMSAGIFLVLLGLVWFAKPPFGAGGGGGGAH

SEQ ID NO: 43 - protein, 394 aa, Escherichia coli
MKRQRNVNLLLMLVLLVAVGQMAQTIYIPAIADMARDLNVREGAVQSVMGAYLLTYGVSQLFYGPISDRVGRRPVILVGMSIFMLATLVAVTTSSLTVLIAASAMQGMGTGVGGVMARTLPRDLYERTQLRHANSLLNMGILVSPLLAPLIGGLLDTMWNWRACYLFLLVLCAGVTFSMARWMPETRPVDAPRTRLLTSYKTLFGNSGFNCYLLMLIGGLAGIAAFEACSGVLMGAVLGLSSMTVSILFILPIPAAFFGAWFAGRPNKRFSTLMWQSVICCLLAGLLMWIPDWFGVMNVWTLLVPAALFFFGAGMLFPLATSGAMEPFPFLAGTAGALVGGLQNIGSGVLASLSAMLPQTGQGSLGLLMTLMGLLIVLCWLPLATRMSHQGQPV

SEQ ID NO: 44 - protein, 512 aa, Escherichia coli
MAITKSTPAPLTGGTLWCVTIALSLATFMQMLDSTISNVAIPTISGFLGASTDEGTWVITSFGVANAIAIPVTGRLAQRIGELRLFLLSVTFFSLSSLMCSLSTNLDVLIFFRVVQGLMAGPLIPLSQSLLLRNYPPEKRTFALALWSMTVIIAPICGPILGGYICDNFSWGWIFLINVPMGIIVLTLCLTLLKGRETETSPVKMNLPGLTLLVLGVGGLQIMLDKGRDLDWFNSSTIIILTVVSVISLISLVIWESTSENPILDLSLFKSRNFTIGIVSITCAYLFYSGAIVLMPQLLQETMGYNAIWAGLAYAPIGIMPLLISPLIGRYGNKIDMRLLVTFSFLMYAVCYYWRSVTFMPTIDFTGIILPQFFQGFAVACFFLPLTTISFSGLPDNKFANASSMSNFFRTLSGSVGTSLTMTLWGRRESLHHSQLTATIDQFNPVFNSSSQIMDKYYGSLSGVLNEINNEITQQSLSISANEIFRMAAIAFILLTVLVWFAKPPFTAKGVG

SEQ ID NO: 45 - protein, 512 aa, Escherichia coli; identical to SEQ ID NO: 44

SEQ ID NO: 46 - protein, 444 aa, Escherichia coli
MILDTVDEKKKGVHTRYLILLIIFIVTAVNYADRATLSIAGTEVAKELQLSAVSMGYIFSAFGWAYLLMQIPGGWLLDKFGSKKVYTYSLFFWSLFTFLQGFVDMFPLAWAGISMFFMRFMLGFSEAPSFPANARIVAAWFPTKERGTASAIFNSAQYFSLALFSPLLGWLTFAWGWEHVFTVMGVIGFVLTALWIKLIHNPTDHPRMSAEELKFISENGAVVDMDHKKPGSAAASGPKLHYIKQLLSNRMMLGVFFGQYFINTITWFFLTWFPIYLVQEKGMSILKVGLVASIPALCGFAGGVLGGVFSDYLIKRGLSLTLARKLPIVLGMLLASTIILCNYTNNTTLVVMLMALAFFGKGFGALGWPVISDTAPKEIVGLCGGVFNVFGNVASIVTPLVIGYLVSELHSFNAALVFVGCSALMAMVCYLFVVGDIKRMELQK

SEQ ID NO: 47 - protein, 450 aa, Escherichia coli
MSSLSQAASSVEKRTNARYWIVVMLFIVTSFNYGDRATLSIAGSEMAKDIGLDPVGMGYVFSAFSWAYVIGQIPGGWLLDRFGSKRVYFWSIFIWSMFTLLQGFVDIFSGFGIIVALFTLRFLVGLAEAPSFPGNSRIVAAWFPAQERGTAVSIFNSAQYFATVIFAPIMGWLTHEVGWSHVFFFMGGLGIVISFIWLKVIHEPNQHPGVNKKELEYIAAGGALINMDQQNTKVKVPFSVKWGQIKQLLGSRMMIGVYIGQYCINALTYFFITWFPVYLVQARGMSILKAGFVASVPAVCGFIGGVLGGIISDWLMRRTGSLNIARKTPIVMGMLLSMVMVFCNYVNVEWMIIGFMALAFFGKGIGALGWAVMADTAPKEISGLSGGLFNMFGNISGIVTPIAIGYIVGTTGSFNGALIYVGVHALIAVLSYLVLVGDIKRIELKPVAGQ

SEQ ID NO: 48 - protein, 475 aa, Escherichia coli
MSDKKKRSMAGLPWIAAMAFFMQALDATILNTALPAIAHSLNRSPLAMQSAIISYTLTVAMLIPVSGWLADRFGTRRIFTLAVSLFTLGSLACALSNSLPQLVVFRVIQGIGGAMMMPVARLALLRAYPRNELLPVLNFVAMPGLVGPILGPVLGGVLVTWATWHWIFLINIPIGIAGLLYARKHMPNFTTARRRFDITGFLLFGLSLVLFSSGIELFGEKIVASWIALTVIVTSIGLLLLYILHARRTPNPLISLDLFKTRTFSIGIVGNIATRLGTGCVPFLMPLMLQVGFGYQAFIAGCMMAPTALGSIIAKSMVTQVLRRLGYRHTLVGITVIIGLMIAQFSLQSPAMAIWMLILPLFILGMAMSTQFTAMNTITLADLTDDNASSGNSVLAVTQQLSISLGVAVSAAVLRVYEGMEGTTTVEQFHYTFITMGIITVASAAMFMLLKTTDGNNLIKRQRKSKPNRVPSESE

SEQ ID NO: 49 - protein, 212 aa, Escherichia coli
MFAEYGVLNYWTYLVGAIFIVLVPGPNTLFVLKNSVSSGMKGGYLAACGVFIGDAVLMFLAWAGVATLIKTTPILFNIVRYLGAFYLLYLGSKILYATLKGKNSEAKSDEPQYGAIFKRALILSLTNPKAILFYVSFFVQFIDVNAPHTGISFFILAATLELVSFCYLSFLIISGAFVTQYIRTKKKLAKVGNSLIGLMFVGFAARLATLQS

SEQ ID NO: 50 - protein, 593 aa, Escherichia coli
MRSFSQLWPTLKRLLAYGSPWRKPLGIAVLMMWVAAAAEVSGPLLISYFIDNMVAKNNLPLKVVAGLAAAYVGLQLFAAGLHYAQSLLFNRAAVGVVQQLRTDVMDAALRQPLSEFDTQPVGQVISRVTNDTEVIRDLYVTVVATVLRSAALVGAMLVAMFSLDWRMALVAIMIFPVVLVVMVIYQRYSTPIVRRVRAYLADINDGFNEIINGMSVIQQFRQQARFGERMGEASRSHYMARMQTLRLDGFLLRPLLSLFSSLILCGLLMLFGFSASGTIEVGVLYAFISYLGRLNEPLIELTTQQAMLQQAVVAGERVFELMDGPRQQYGNDDRPLQSGTIEVDNVSFAYRDDNLVLKNINLSVPSRNFVALVGHTGSGKSTLASLLMGYYPLTEGEIRLDGRPLSSLSHSALRQGVAMVQQDPVVLADTFLANVTLGRDISEERVWQALETVQLAELARSMSDGIYTPLGEQGNNLSVGQKQLLALARVLVETPQILILDEATASIDSGTEQAIQHALAAVREHTTLVVIAHRLSTIVDADTILVLHRGQAVEQGTHQQLLAAQGRYWQMYQLQLAGEELAASVREEESLSA

SEQ ID NO: 51 - protein, 471 aa, Escherichia coli
MTDLPDSTRWQLWIVAFGFFMQSLDTTIVNTALPSMAQSLGESPLHMHMVIVSYVLTVAVMLPASGWLADKVGVRNIFFTAIVLFTLGSLFCALSGTLNELLLARALQGVGGAMMVPVGRLTVMKIVPREQYMAAMTFVTLPGQVGPLLGPALGGLLVEYASWHWIFLINIPVGIIGAIATLLLMPNYTMQTRRFDLSGFLLLAVGMAVLTLALDGSKGTGLSPLTIAGLVAVGVVALVLYLLHARNNNRALFSLKLFRTRTFSLGLAGSFAGRIGSGMLPFMTPVFLQIGLGFSPFHAGLMMIPMVLGSMGMKRIVVQVVNRFGYRRVLVATTLGLSLVTLLFMTTALLGWYYVLPFVLFLQGMVNSTRFSSMNTLTLKDLPDNLASSGNSLLSMIMQLSMSIGVTIAGLLLGLFGSQHVSVDSGTTQTVFMYTWLSMALIIALPAFIFARVPNDTHQNVAISRRKRSAQ

SEQ ID NO: 52 - protein, 408 aa, Escherichia coli
MSPCENDTPINWKRNLIVAWLGCFLTGAAFSLVMPFLPLYVEQLGVTGHSALNMWSGIVFSITFLFSAIASPFWGGLADRKGRKLMLLRSALGMGIVMVLMGLAQNIWQFLILRALLGLLGGFVPNANALIATQVPRNKSGWALGTLSTGGVSGALLGPMAGGLLADSYGLRPVFFITASVLILCFFVTLFCIREKFQPVSKKEMLHMREVVTSLKNPKLVLSLFVTTLIIQVATGSIAPILTLYVRELAGNVSNVAFISGMIASVPGVAALLSAPRLGKLGDRIGPEKILITALIFSVLLLIPMSYVQTPLQLGILRFLLGAADGALLPAVQTLLVYNSSNQIAGRIFSYNQSFRDIGNVTGPLMGAAISANYGFRAVFLVTAGVVLFNAVYSWNSLRRRRIPQVSN

SEQ ID NO: 53 - protein, 391 aa, Escherichia coli
MSRFLICSFALVLLYPAGIDMYLVGLPRIAADLNASEAQLHIAFSVYLAGMAAAMLFAGKVADRSGRKPVAIPGAALFIIASVFCSLAETSTLFLAGRFLQGLGAGCCYVVAFAILRDTLDDRRRAKVLSLLNGITCIIPVLAPVLGHLIMLKFPWQSLFWAMAMMGIAVLMLSLFILKETRPAAPAASDKPRENSESLLNRFFLSRVVITTLSVSVILTFVNTSPVLLMEIMGFERGEYATIMALTAGVSMTVSFSTPFALGIFKPRTLMITSQVLFLAAGITLAVSPSHAVSLFGITLICAGFSVGFGVAMSQALGPFSLRAGVASSTLGIAQVCGSSLWIWLAAVVGIGAWNMLIGILIACSIVSLLLIMFVAPGRPVAAHEEIHHHA

SEQ ID NO: 54 - protein, 410 aa, Escherichia coli
MPRFFTRHAATLFFPMALILYDFAAYLSTDLIQPGIINVVRDFNADVSLAPAAVSLYLAGGMALQWLLGPLSDRIGRRPVLITGALIFTLACAATMFTTSMTQFLIARAIQGTSICFIATVGYVTVQEAFGQTKGIKLMAIITSIVLIAPIIGPLSGAALMHFMHWKVLFAIIAVMGFISFVGLLLAMPETVKRGAVPFSAKSVLRDFRNVFCNRLFLFGAATISLSYIPMMSWVAVSPVILIDAGSLTTSQFAWTQVPVFGAVIVANAIVARFVKDPTEPRFIWRAVPIQLVGLSLLIVGNLLSPHVWLWSVLGTSLYAFGIGLIFPTLFRFTLFSNKLPKGTVSASLNMVILMVMSVSVEIGRWLWFNGGRLPFHLLAVVAGVIVVFTLAGLLNRVRQHQAAELVEEQ

SEQ ID NO: 55 - protein, 403 aa, Escherichia coli
MSTRTPSSSSSRLMLTIGLCFLVALMEGLDLQAAGIAAGGIAQAFALDKMQMGWIFSAGILGLLPGALVGGMLADRYGRKRILIGSVALFGLFSLATAIAWDFPSLVFARLMTGVGLGAALPNLIALTSEAAGPRFRGTAVSLMYCGVPIGAALAATLGFAGANLAWQTVFWVGGVVPLILVPLLMRWLPESAVFAGEKQSAPPLRALFAPETATATLLLWLCYFFTLLVVYMLINWLPLLLVEQGFQPSQAAGVMFALQMGAASGTLMLGALMDKLRPVTMSLLIYSGMLASLLALGTVSSFNGMLLAGFVAGLFATGGQSVLYALAPLFYSSQIRATGVGTAVAVGRLGAMSGPLLAGKMLALGTGTVGVMAASAPGILVAGLAVFILMSRRSRIQPCADA

SEQ ID NO: 56 - protein, 295 aa, Escherichia coli
MPGSLRKMPVWLPIVILLVAMASIQGGASLAKSLFPLVGAPGVTALRLALGTLILIAFFKPWRLRFAKEQRLPLLFYGVSLGGMNYLFYLSIQTVPLGIAVALEFTGPLAVALFSSRRPVDFVWVVLAVLGLWFLLPLGQDVSHVDLTGCALALGAGACWAIYILSGQRAGAEHGPATVAIGSLIAALIFVPIGALQAGEALWHWSVIPLGLAVAILSTALPYSLEMIALTRLPTRTFGTLMSMEPALAAVSGMIFLGETLTPIQLLALGAIIAASMGSTLTVRKESKIKELDIN

SEQ ID NO: 57 - protein, 206 aa, Escherichia coli
MTLEWWFAYLLTSIILSLSPGSGAINTMTTSLNHGYRGAVASIAGLQTGLAIHIVLVGVGLGTLFSRSVIAFEVLKWAGAAYLIWLGIQQWRAAGAIDLKSLASTQSRRHLFQRAVFVNLTNPKSIVFLAALFPQFIMPQQPQLMQYIVLGVTTIVVDIIVMIGYATLAQRIALWIKGPKQMKALNKIFGSLFMLVGALLASARHA

SEQ ID NO: 58 - protein, 206 aa, Escherichia coli
MLMLFLTVAMVHIVALMSPGPDFFFVSQTAVSRSRKEAMMGVLGITCGVMVWAGIALLGLHLIIEKMAWLHTLIMVGGGLYLCWMGYQMLRGALKKEAVSAPAPQVELAKSGRSFLKGLLTNLANPKAIIYFGSVFSLFVGDNVGTTARWGIFALIIVETLAWFTVVASLFALPQMRRGYQRLAKWIDGFAGALFAGFGIHLIISR

SEQ ID NO: 59 - protein, 223 aa, Escherichia coli
MMQLVHLFMDEITMDPLHAVYLTVGLFVITFFNPGANLFVVVQTSLASGRRAGVLTGLGVALGDAFYSGLGLFGLATLITQCEEIFSLIRIVGGAYLLWFAWCSMRRQSTPQMSTLQQPISAPWYVFFRRGLITDLSNPQTVLFFISIFSVTLNAETPTWARLMAWAGIVLASIIWRVFLSQAFSLPAVRRAYGRMQRVASRVIGAIIGVFALRLIYEGVTQR

SEQ ID NO: 60 - protein, 454 aa, Escherichia coli
MNDYKMTPGERRATWGLGTVFSLRMLGMFMVLPVLTTYGMALQGASEALIGIAIGIYGLTQAVFQIPFGLLSDRIGRKPLIVGGLAVFAAGSVIAALSDSIWGIILGRALQGSGAIAAAVMALLSDLTREQNRTKAMAFIGVSFGITFAIAMVLGPIITHKLGLHALFWMIAILATTGIALTIWVVPNSSTHVLNRESGMVKGSFSKVLAEPRLLKLNFGIMCLHILLMSTFVALPGQLADAGFPAAEHWKVYLATMLIAFGSVVPFIIYAEVKRKMKQVFVFCVGLIVVAEIVLWNAQTQFWQLVVGVQLFFVAFNLMEALLPSLISKESPAGYKGTAMGVYSTSQFLGVAIGGSLGGWINGMFDGQGVFLAGAMLAAVWLTVASTMKEPPYVSSLRIEIPANIAANEALKVRLLETEGIKEVLIAEEEHSAYVKIDSKVTNRFEIEQAIRQA

SEQ ID NO: 61 - protein, 804 aa, Escherichia coli
MIARWFWREWRSPSLLIVWLALSLAVACVLALGNISDRMEKGLSQQSREFMAGDRALRSSREVPQAWLEEAQKRGLKVGKQLTFATMTFAGDTPQLANVKAVDDIYPMYGDLQTNPPGLKPQAGSVLLAPRLMALLNLKTGDTIDVGDATLRIAGEVIQEPDSGFNPFQMAPRLMMNLADVDKTGAVQPGSRVTWRYKFGGNENQLDGYEKWLLPQLKPEQRWYGLEQDEGALGRSMERSQQFLLLSALLTLLLAVAAVAVAMNHYCRSRYDLVAILKTLGAGRAQLRKLIVGQWLMVLTLSAVTGGAIGLLFENVLMVLLKPVLPAALPPASLWPWLWALGTMTVISLLVGLRPYRLLLATQPLRVLRNDVVANVWPLKFYLPIVSVVVVLLLAGLMGGSMLLWAVLAGAVVLALLCGVLGWMLLNVLRRMTLKSLPLRLAVSRLLRQPWSTLSQLSAFSLSFMLLALLLVLRGDLLDRWQQQLPPESPNYFLINIATEQVAPLKAFLAEHQIVPESFYPVVRARLTAINDKPTEGNEDEALNRELNLTWQNTRPDHNPIVAGNWPPKADEVSMEEGLAKRLNVALGDTVTFMGDTQEFRAKVTSLRKVDWESLRPNFYFIFPEGALDGQPQSWLTSFRWENGNGMLTQLNRQFPTISLLDIGAILKQVGQVLEQVSRALEVMVVLVTACGMLLLLAQVQVGMRQRHQELVVWRTLGAGKKLLRTTLWCEFAMLGFVSGLVAAIGAETALAVLQAKVFDFPWEPDWRLWIVLPCSGALLLSLFGGWLGARLVKGKALFRQFAG

SEQ ID NO: 62 - protein, 295 aa, Escherichia coli; identical to SEQ ID NO: 56

SEQ ID NO: 63 - protein, 402 aa, Escherichia coli
MTVNSSRNALKRRTWALFMFFFLPGLLMASWATRTPAIRDILSVSIAEMGGVLFGLSIGSMSGILCSAWLVKRFGTRNVILVTMSCALIGMMILSLALWLTSPLLFAVGLGVFGASFGSAEVAINVEGAAVEREMNKTVLPMMHGFYSLGTLAGAGVGMALTAFGVPATVHILLAALVGIAPIYIAIQAIPDGTGKNAADGTQHGEKGVPFYRDIQLLLIGVVVLAMAFAEGSANDWLPLLMVDGHGFSPTSGSLIYAGFTLGMTVGRFTGGWFIDRYSRVAVVRASALMGALGIGLIIFVDSAWVAGVSVVLWGLGASLGFPLTISAASDTGPDAPTRVSVVATTGYLAFLVGPPLLGYLGEHYGLRSAMLVVLALVILAAIVAKAVAKPDTKTQTAMENS

SEQ ID NO: 64 - protein, 230 aa, Escherichia coli
MKAFDLHRMAFDKVPFDFLGEVALRSLYTFVLVFLFLKMTGRRGVRQMSLFEVLIILTLGSAAGDVAFYDDVPMVPVLIVFITLALLYRLVMWLMAHSEKLEDLLEGKPVVIIEDGELAWSKLNNSNMTEFEFFMELRLRGVEQLGQVRLAILETNGQISVYFFEDDKVKPGLLILPSDCTQRYKVVPESADYACIRCSEIIHMKAGEKQLCPRCANPEWTKASRAKRVT

SEQ ID NO: 65 - protein, 391 aa, Escherichia coli
MRLFSIPPPTLLAGFLAVLIGYASSAAIIWQAAIVAGATTAQISGWMTALGLAMGVSTLTLTLWYRVPVLTAWSTPGAALLVTGLQGLTLNEAIGVFIVTNALIVLCGITGLFARLMRIIPHSLAAAMLAGILLRFGLQAFASLDGQFTLCGSMLLVWLATKAVAPRYAVIAAMIIGIVIVIAQGDVVTTDVVFKPVLPTYITPDFSFAHSLSVALPLFLVTMASQNAPGIAAMKAAGYSAPVSPLIVFTGLLALVFSPFGVYSVGIAAITAAICQSPEAHPDKDQRWLAAAVAGIFYLLAGLFGSAITGMMAALPVSWIQMLAGLALLSTIGGSLYQALHNERERDAAVVAFLVTASGLTLVGIGSAFWGLIAGGVCYVVLNLIADRNRY

SEQ ID NO: 66 - protein, 293 aa, Escherichia coli
MTRQKATLIGLIAIVLWSTMVGLIRGVSEGLGPVGGAAAIYSLSGLLLIFTVGFPRIRQIPKGYLLAGSLLFVSYEICLALSLGYAATHHQAIEVGMVNYLWPSLTILFAILFNGQKTNWLIVPGLLLALVGVCWVLGGDNGLHYDEIINNITTSPLSYFLAFIGAFIWAAYCTVTNKYARGFNGITVFVLLTGASLWVYYFLTPQPEMIFSTPVMIKLISAAFTLGFAYAAWNVGILHGNVTIMAVGSYFTPVLSSALAAVLLSAPLSFSFWQGALMVCGGSLLCWLATRRG

SEQ ID NO: 67 - protein, 299 aa, Escherichia coli; identical to SEQ ID NO: 40

SEQ ID NO: 68 - protein, 109 aa, Escherichia coli
MAQFEWVHAAWLALAIVLEIVANVFLKFSDGFRRKIFGLLSLAAVLAAFSALSQAVKGIDLSVAYALWGGFGIAATLAAGWILFGQRLNRKGWIGLVLLLAGMIMVKLA

SEQ ID NO: 69 - protein, 403 aa, Escherichia coli
MQPGKRFLVWLAGLSVLGFLATDMYLPAFAAIQADLQTPASAVSASLSLFLAGFAAAQLLWGPLSDRYGRKPVLLIGLTIFALGSLGMLWVENAATLLVLRFVQAVGVCAAAVIWQALVTDYYPSQKVNRIFAAIMPLVGLSPALAPLLGSWLLVHFSWQAIFATLFAITVVLILPIFWLKPTTKARNNSQDGLTFTDLLRSKTYRGNVLIYAACSASFFAWLTGSPFILSEMGYSPAVIGLSYVPQTIAFLIGGYGCRAALQKWQGKQLLPWLLVLFAVSVIATWAAGFISHVSLVEILIPFCVMAIANGAIYPIVVAQALRPFPHATGRAAALQNTLQLGLCFLASLVVSWLISISTPLLTTTSVMLSTVVLVALGYMMQRCEEVGCQNHGNAEVAHSESH

SEQ ID NO: 70 - protein, 389 aa, Escherichia coli
MKINYPLLALAIGAFGIGTTEFSPMGLLPVIARGVDVSIPAAGMLISAYAVGVMVGAPLMTLLLSHRARRSALIFLMAIFTLGNVLSAIAPDYMTLMLSRILTSLNHGAFFGLGSVVAASVVPKHKQASAVATMFMGLTLANIGGVPAATWLGETIGWRMSFLATAGLGVISMVSLFFSLPKGGAGARPEVKKELAVLMRPQVLSALLTTVLGAGAMFTLYTYISPVLQSITHATPVFVTAMLVLIGVGFSIGNYLGGKLADRSVNGTLKGFLLLLMVIMLAIPFLARNEFGAAISMVVWGAATFAVVPPLQMRVMRVASEAPGLSSSVNIGAFNLGNALGAAAGGAVISAGLGYSFVPVMGAIVAGLALLLVFMSARKQPETVCVANS

SEQ ID NO: 71 - protein, 421 aa, Escherichia coli
MSQNKAFSTPFILAVLCIYFSYFLHGISVITLAQNMSSLAEKFSTDNAGIAYLISGIGLGRLISILFFGVISDKFGRRAVILMAVIMYLLFFFGIPACPNLTLAYGLAVCVGIANSALDTGGYPALMECFPKASGSAVILVKAMVSFGQMFYPMLVSYMLLNNIWYGYGLIIPGILFVLITLMLLKSKFPSQLVDASVTNELPQMNSKPLVWLEGVSSVLFGVAAFSTFYVIVVWMPKYAMAFAGMSEAEALKTISYYSMGSLVCVFIFAALLKKMVRPIWANVFNSALATITAAIIYLYPSPLVCNAGAFVIGFSAAGGILQLGVSVMSEFFPKSKAKVTSIYMMMGGLANFVIPLITGYLSNIGLQYIIVLDFTFALLALITAIIVFIRYYRVFIIPENDVRFGERKFCTRLNTIKHRG

SEQ ID NO: 72 - protein, 404 aa, Escherichia coli
MKNPYFPTALGLYFNYLVHGMGVLLMSLNMASLETLWQTNAAGVSIVISSLGIGRLSVLLFAGLLSDRFGRRPFIMLGMCCYMAFFFGILQTNNIIIAYVFGFLAGMANSFLDAGTYPSLMEAFPRSPGTANILIKAFVSSGQFLLPLIISLLVWAELWFGWSFMIAAGIMFINALFLYRCTFPPHPGRRLPVIKKTTSSTEHRCSIIDLASYTLYGYISMATFYLVSQWLAQYGQFVAGMSYTMSIKLLSIYTVGSLLCVFITAPLIRNTVRPTTLLMLYTFISFIALFTVCLHPTFYVVIIFAFVIGFTSAGGVVQIGLTLMAERFPYAKGKATGIYYSAGSIATFTIPLITAHLSQRSIADIMWFDTAIAAIGFLLALFIGLRSRKKTRHHSLKENVAPGG

SEQ ID NO: 73 - protein, 452 aa, Escherichia coli
MEQYDQIGARLDRLPLARFHYRIFGIISFSLLLTGFLSYSGNVVLAKLVSNGWSNNFLNAAFTSALMFGYFIGSLTGGFIGDYFGRRRAFRINLLIVGIAATGAAFVPDMYWLIFFRFLMGTGMGALIMVGYASFTEFIPATVRGKWSARLSFVGNWSPMLSAAIGVVVIAFFSWRIMFLLGGIGILLAWFLSGKYFIESPRWLAGKGQIAGAECQLREVEQQIEREKSIRLPPLTSYQSNSKVKVIKGTFWLLFKGEMLRRTLVAITVLIAMNISLYTITVWIPTIFVNSGIDVDKSILMTAVIMIGAPVGIFIAALIIDHFPRRLFGSTLLIIIAVLGYIYSIQTTEWAILIYGLVMIFFLYMYVCFASAVYIPELWPTHLRLRGSGFVNAVGRIVAVFTPYGVAALLTHYGSITVFMVLGVMLLLCALVLSIFGIETRKVSLEEISEVN

SEQ ID NO: 74 - protein, 252 aa, Escherichia coli [sequence omitted]

SEQ ID NO: 75 - protein, 278 aa, Escherichia coli
MLADIRYWENDATNKHYAIAHFNVWNAEMLMGVIDAAEEAKSPVIISFGTGFVGNTSFEDFSHMMVSMAQKATVPVITHWDHGRSMEIIHNAWTHGMNSLMRDASAFDFEENIRLTKEAVDFFHPLGIPVEAELGHVGNETVYEEALAGYHYTDPDQAAEFVERTGCDSLAVAIGNQHGVYTSEPQLNFEVVKRVRDAVSVPLVLHGASGISDADIKTAISLGIAKINIHTELCQAAMVAVKENQDQPFLHLEREVRKAVKERALEKIKLFGSDGKAE

SEQ ID NO: 76 - protein, 459 aa, Escherichia coli
MEQITKPHCGARLDRLPDCRWHSSMFAIVAFGLLVCWSNAVGGLILAQLKALGWTDNSTTATFSAITTAGMFLGALVGGIIGDKTGRRNAFILYEAIHIASMVVGAFSPNMDFLIACRFVMGVGLGALLVTLFAGFTEYMPGRNRGTWSSRVSFIGNWSYPLCSLIAMGLTPLISAEWNWRVQLLIPAILSLIATALAWRYFPESPRWLESRGRYQEAEKVMRSIEEGVIRQTGKPLPPVVIADDGKAPQAVPYSALLTGVLLKRVILGSCVLIAMNVVQYTLINWLPTIFMTQGINLKDSIVLNTMSMFGAPFGIFIAMLVMDKIPRKTMGVGLLILIAVLGYIYSLQTSMLLITLIGFFLITFVYMYVCYASAVYVPEIWPTEAKLRGSGLANAVGRISGIAAPYAVAVLLSSYGVTGVFILLGAVSIIVAIAIATIGIETKGVSVESLSIDAVANK

SEQ ID NO: 77 - protein, 393 aa, Escherichia coli
MTCSTSLSGKNRIVLIAGILMIATTLRVTFTGAAPLLDTIRSAYSLTTAQTGLLTTLPLLAFALISPLAAPVARRFGMERSLFAALLLICAGIAIRSLPSPYLLFGGTAVIGGGIALGNVLLPGLIKRDFPHSVARLTGAYSLTMGAAAALGSAMVVPLALNGFGWQGALLMLMCFPLLALFLWLPQWRSQQHANLSTSRALHTRGIWRSPLAWQVTLFLGINSLVYYVIIGWLPAILISHGYSEAQAGSLHGLLQLATAAPGLLIPLFLHHVKDQRGIAAFVALMCAVGAVGLCFMPAHAITWTLLFGFGSGATMILGLTFIGLRASSAHQAAALSGMAQSVGYLLAACGPPLMGKIHDANGNWSVPLMGVAILSLLMAIFGLCAGRDKEIR

SEQ ID NO: 78 - protein, 212 aa, Escherichia coli; identical to SEQ ID NO: 49

SEQ ID NO: 79 - protein, 427 aa, Escherichia coli
MALNTPQITPTKKITVRAIGEELPRGDYQRCPQCDMLFSLPEINSHQSAYCPRCQAKIRDGRDWSLTRLAAMAFTMLLLMPFAWGEPLLHIWLLGIRIDANVMQGIWQMTKQGDAITGSMVFFCVIGAPLILVTSIAYLWFGNRLGMNLRPVLLMLERLKEWVMLDIYLVGIGVASIKVQDYAHIQAGVGLFSFVALVILTTVTLSHLNVEELWERFYPQRPATRRDEKLRVCLGCHFTGYPDQRGRCPRCHIPLRLRRRHSLQKCWAALLASIVLLLPANLLPISIIYLNGGRQEDTILSGIMSLASSNIAVAGIVFIASILVPFTKVIVMFTLLLSIHFKCQQGLRTRILLLRMVTWIGRWSMLDLFVISLTMSLINRDQILAFTMGPAAFYFGAAVILTILAVEWLDSRLLWDAHESGNARFDD

SEQ ID NO: 80 - protein, 306 aa, Escherichia coli
MRFRQLLPLFGALFALYIIWGSTYFVIRIGVESWPPLMMAGVRFLAAGILLLAFLLLRGHKLPPLRPLLNAALIGLLLLAVGNGMVTVAEHQNVPSGIAAVVVATVPLFTLCFSRLFGIKTRKLEWVGIAIGLAGIIMLNSGGNLSGNPWGAILILIGSISWAFGSVYGSRITLPVGMMAGAIEMLAAGVVLMIASMIAGEKLTALPSLSGFLAVGYLALFGSIIAINAYMYLIRNVSPALATSYAYVNPVVAVLLGTGLGGETLSKIEWLALGVIVFAVVLVTLGKYLFPAKPVVAPVIQDASSE

SEQ ID NO: 81-89 - skipped (9 entries)

SEQ ID NO: 90 - protein, 547 aa, Escherichia coli
MLRHILTAKNLLSNPIFKFPNCLPFLSTVCCICRQFVGENLCSFADSPSLFEMWFHFLQLRSALNISSALRQVVHGTRWHAKRKSYKVLFWREITPLAVPIFMENACVLLMGVLSTFLVSWLGKDAMAGVGLADSFNMVIMAFFAAIDLGTTVVVAFSLGKRDRRRARVATRQSLVIMTLFAVLLATLIHHFGEQIIDFVAGDATTEVKALALTYLELTVLSYPAAAITLIGSGALRGAGNTKIPLLINGSLNILNIIISGILIYGLFSWPGLGFVGAGLGLTISRYIGAVAILWVLAIGFNPALRISLKSYFKPLNFSIIWEVMGIGIPASVESVLFTSGRLLTQMFVAGMGTSVIAGNFIAFSIAALINLPGSALGSASTIITGRRLGVGQIAQAEIQLRHVFWLSTLGLTAIAWLTAPFAGVMASFYTQDPQVKHVVVILIWLNALFMPIWSASWVLPAGFKGARDARYAMWVSMLSMWGCRVVVGYVLGIMLGWGVVGVWMGMFADWAVRAVLFYWRMVTGRWLWKYPRPEPQKCEKKPVVSE

SEQ ID NO: 91 - protein, 527 aa, Escherichia coli
MEWIADPSIWAGLITLIVIELVLGIDNLVFIAILAEKLPPKQRDRARVTGLLLAMLMRLLLLASISWLVTLTQPLFSFRSFTFSARDLIMLFGGFFLLFKATMELNERLEGKDSNNPTQRKGAKFWGVVTQIVVLDAIFSLDSVITAVGMVDHLLVMMAAVVIAISLMLMASKPLTQFVNSHPTIVILCLSFLLMIGFSLVAEGFGFVIPKGYLYAAIGFSVMIEALNQLAIFNRRRFLSANQTLRQRTTEAVMRLLSGQKEDAELDAETASMLVDHGNQQIFNPQERRMIERVLNLNQRTVSSIMTSRHDIEHIDLNAPEEEIRQLLERNQHTRLVVTDGDDAEDLLGVVHVIDLLQQSLRGEPLNLRVLIRQPLVFPETLPLLPALEQFRNARTHFAFVVDEFGSVEGIVTLSDVTETIAGNLPNEVEEIDARHDIQKNADGSWTANGHMPLEDLVQYVPLPLDEKREYHTIAGLLMEYLQRIPKPGEEVQVGDYLLKTLQVESHRVQKVQIIPLRKDGEMEYEV

SEQ ID NO: 92 - protein, 211 aa, Escherichia coli
MFSYYFQGLALGAAMILPLGPQNAFVMNQGIRRQYHIMIALLCAISDLVLICAGIFGGSALLMQSPWLLALVTWGGVAFLLWYGFGAFKTAMSSNIELASAEVMKQGRWKIIATMLAVTWLNPHVYLDTFVVLGSLGGQLDVEPKRWFALGTISASFLWFFGLALLAAWLAPRLRTAKAQRIINLVVGCVMWFIALQLARDGIAHAQALFS

SEQ ID NO: 93 - protein, 392 aa, Escherichia coli [sequence omitted]

SEQ ID NO: 94 - protein, 195 aa, Escherichia coli; identical to SEQ ID NO: 41

SEQ ID NO: 95 - protein, 440 aa, Escherichia coli
MQATATTLDHEQEYTPINSRNKVLVASLIGTAIEFFDFYIYATAAVIVFPHIFFPQGDPTAATLQSLATFAIAFVARPIGSAVFGHFGDRVGRKATLVASLLTMGISTVVIGLLPGYATIGIFAPLLLALARFGQGLGLGGEWGGAALLATENAPPRKRALYGSFPQLGAPIGFFFANGTFLLLSWLLTDEQFMSWGWRVPFIFSAVLVIIGLYVRVSLHESPVFEKVAKAKKQVKIPLGTLLTKHVRVTVLGTFIMLATYTLFYIMTVYSMTFSTAAAPVGLGLPRNEVLWMLMMAVIGFGVMVPVAGLLADAFGRRKSMVIITTLIILFALFAFNPLLGSGNPILVFAFLLLGLSLMGLTFGPMGALLPELFPTEVRYTGASFSYNVASILGASVAPYIAAWLQTNYGLGAVGLYLAAMAGLTLIALLLTHETRHQSL

SEQ ID NO: 96 - protein, 553 aa, Escherichia coli
MSDIALTVSILALVAVVGLFIGNVKFRGIGLGIGGVLFGGIIVGHFVSQAGMTLSSDMLHVIQEFGLILFVYTIGIQVGPGFFASLRVSGLRLNLFAVLIVIIGGLVTAILHKLFDIPLPVVLGIFSGAVTNTPALGAGQQILRDLGTPMEMVDQMGMSYAMAYPFGICGILFTMWMLRVIFRVNVETEAQQHESSRTNGGALIKTINIRVENPNLHDLAIKDVPILNGDKIICSRLKREETLKVPSPDTIIQLGDLLHLVGQPADLHNAQLVIGQEVDTSLSTKGTDLRVERVVVTNENVLGKRIRDLHFKERYDVVISRLNRAGVELVASGDISLQFGDILNLVGRPSAIDAVANVLGNAQQKLQQVQMLPVFIGIGLGVLLGSIPVFVPGFPAALKLGLAGGPLIMALILGRIGSIGKLYWFMPPSANLALRELGIVLFLSVVGLKSGGDFVNTLVNGEGLSWIGYGALITAVPLITVGILARMLAKMNYLTMCGMLAGSMTDPPALAFANNLHPTSGAAALSYATVYPLVMFLRIITPQLLAVLFWSIG

SEQ ID NO: 97 - protein, 206 aa, Escherichia coli; identical to SEQ ID NO: 57

SEQ ID NO: 98 - protein, 206 aa, Escherichia coli; identical to SEQ ID NO: 58

SEQ ID NO: 99 - protein, 301 aa, Escherichia coli
MSAAGKSNPLAISGLVVLTLIWSYSWIFMKQVTSYIGAFDFTALRCIFGALVLFIVLLLRGRGMRPTPFKYTLAIALLQTCGMVGLAQWALVSGGAGKVAILSYTMPFWVVIFAALFLGERLRRGQYFAILIAAFGLFLVLQPWQLDFSSMKSAMLAILSGVSWGASAIVAKRLYARHPRVDLLSLTSWQMLYAALVMSVVALLVPQREIDWQPTVFWALAYSAILATALAWSLWLFVLKNLPASIASLSTLAVPVCGVLFSWWLLGENPGAVEGSGIVLIVLALALVSRKKKEAVSVKRI

SEQ ID NO: 100 - protein, 184 aa, Escherichia coli
MAKYNEKELADTSKFLSFVLRHKPEAIGIVLDREGWADIDKLILCAQKAGKRLTRALLDTVVATSDKKRFSYSSDGRCIRAVQGHSTSQVAISFAEKTPPQFLYHGTASRFLDEIKKQGLIAGERHYVHLSADEATARKVGARHGSPVILTVKAQEMAKRGLPFWQAENGVWLTSTVAVEFLEW

SEQ ID NO: 101 - protein, 392 aa, Escherichia coli
MPSSTHPVERFSFSTALFGMLVLTLGMGLGRFLYTPMLPVMMAEGSFSFSQLSWIASGNYAGYLAGSLLFSFGAFHQPSRLRPFLLASALASGLLILAMAWLPPFILVLLIRVLAGVASAGMLIFGSTLIMQHTRHPFVLAALFSGVGIGIALGNEYVLAGLHFDLSSQTLWQGAGALSGMMLIALTLLMPSKKHAITPMPLAKTEQQIMSWWLLAILYGLAGFGYIIVATYLPLMAKDAGSPLLTAHLWTLVGLSIVPGCFGWLWAAKRWGALPCLTANLLVQAICVLLTLASDSPLLLIISSLGFGGTFMGTTSLVMTIARQLSVPGNLNLLGFVTLIYGIGQILGPALTSMLSNGTSALASATLCGAAALFIAALISTVQLFKLQVVTS

SEQ ID NO: 102 - protein, 410 aa, Escherichia coli; identical to SEQ ID NO: 54

SEQ ID NO: 103 - protein, 222 aa, Escherichia coli
MREQIKQDIDLIEILFYLKKKIRVILFIMAICMAMVLLFLYINKDNIKVIYSLKINQTTPGILVSCDSNNNFACQTTMTEDVIQRITTFFHTSPDVKNREIRLEWSGDKRALPTAEEEISRVQASIIKWYASEYHNGRQVLDEIQTPSAINSELYTKMIYLTRNWSLYPNGDGCVTISSPEIKNKYPAAICLALGFFLSIVISVMFCLVKKMVDEYQQNSGQ

SEQ ID NO: 104 - protein, 288 aa, Escherichia coli
MQRLEQRSPDAILLLFLIAQTVDITMPVFALLALVAYSVSLALIVPGLLQKNGGWRRMAIISAVIALVCHAIALEARILPDGDSGQNLSLLNVGSLVSLMICTVMTIVASRNRGWLLLPIVYAFALINLALATFMPNEYITHLEATPGMLVHIGLSLFSYATLIIAALYALQLAWIDYQLKNKKLAFNQEMPPLMSIERKMFHITQIGVVLLTLTLCTGLFYMHNLFSMENIDKAVLSIVAWFVYIVLLWGHYHEGWRGRRVVWFNVAGAVILTLAYFGSRIVQQLIS

SEQ ID NO: 105 - protein, 324 aa, Escherichia coli
MPVMISGVLYALLAGLMWGLIFVGPLIVPEYPAMLQSMGRYLALGLIALPIAWLGRVRLRQLARRDWLTALMLTMMGNLIYYFCLASAIQRTGAPVSTMIIGTLPVVIPVFANLLYSQRDGKLAWGKLAPALICIGIGLACVNIAELNHGLPDFDWARYTSGIVLALVSVVCWAWYALRNARWLRENPDKHPMMWATAQALVTLPVSLIGYLVACYWLNTQTPDFSLPFGPRPLVFISLMVAIAVLCSWVGALCWNVASQLLPTVILGPLIVFETLAGLLYTFLLRQQMPPLMTLSGIALLVIGVVIAVRAKPEKPLTESVSES

SEQ ID NO: 106 - protein, 447 aa, Escherichia coli
MLNSILVILCLIAVSAFFSMSEISLAASRKIKLKLLADEGNINAQRVLNMQENPGMFFTVVQIGLNAVAILGGIVGDAAFSPAFHSLFSRYMSAELSEQLSFILSFSLVTGMFILFADLTPKRIGMIAPEAVALRIINPMRFCLYVCTPLVWFFNGLANIIFRIFKLPMVRKDDITSDDIYAVVEAGALAGVLRKQEHELIENVFELESRTVPSSMTPRENVIWFDLHEDEQSLKNKVAEHPHSKFLVCNEDIDHIIGYVDSKDLLNRVLANQSLALNSGVQIRNTLIVPDTLTLSEALESFKTAGEDFAVIMNEYALVVGIITLNDVMTTLMGDLVGQGLEEQIVARDENSWLIDGGTPIDDVMRVLDIDEFPQSGNYETIGGFMMFMLRKIPKRTDSVKFAGYKFEVVDIDNYRIDQLLVTRIDSKATALSPKLPDAKDKEESVA

SEQ ID NO: 107 - protein, 1219 aa, Chloroflexus aggregans
MSVTGRLAGKIALITGGAGNIGSEMTRRFLAEGATVIISGRNSAKLAALAERLRSEAGVPAKRIDLEVMDGSDPAAVRAGVAAIIGRHGHIDILVNNAGSTGAQRRLAEIPLNETDRDLDDEEALSTSVANLLGMAWHLMRILSPHMPPGSAIINISTIFSRAEYYGRIPYVVPKAALNTLTQIAARELGIRGIRVNTIFPGPIESERIQTVFQRMDQLKGRPEGDTASQFLATMRLYRANDQGQLERRFPTICDVADAAVFLASDEAAALTGETIEVTHGMELPTSSETSLLARTDLRTIDANGRTTLICAGDQIEEVMALTGMLRTCGSEVIIGFRSEAALAQFEQAIGESRRLAGESFIPPIALPIDLRNPSTIDALFDWAGENTGGIHAAVILPASGREPATQVIDIDDAHVQAFLNDEIVGSIIIASRLARYWQAQRIAPGARAREPRVIFLSNGASTAGNPYGRIQSAAIEQLIRVWRHEAALDYERATAAGERVLPAVWASQIVRFANRSLEGLEFACAWTAQLLHSQRRINEITLTIPADISATTGARSASVGWAESLIGLHLGKVALITGGSAGIGGQIGRLLALSGARVMLAARDPHKLEQIQATIRAELAEVGYTDVEERVQIAPGCDVSSEEQLVDLVERTLAAFGTVDYLINNAGIAGVEEMVIDMPVEGWRNTLYANLISNYSLMRKLAPLMKKQGSGYVLNVSSYFGGEKDAAIPYPNRADYAVSKAGQRAMAEVFARFLGPEIQINAIAPGPVEGDRLRGTGERPGLFARRARLILENKRLNELHAALITAARTDNRPMRELVELLLPNDVAALAQHPAAPDVLRTLAKRFQSEGDPAASSSSFLLNRSIAAKLLARLINGGYDLPADIFANLAVPPDPFFTRAQIDREARKVRDGIMGMLYLQRMPTEFDVAMATVYYLADRNVSGETFHPSGGLRYERTPTGGELFGLPAPERLAELVGSTVYLIGEHLTEHLNLLARAYLERYGARQVVMIVETEAGAEKMRHLLHDHVEAGRLPIIVAGDQIEAAIDQAIANYGRPGPVVCTPFRPLPSAPLVGRKDSDWSTVLSEAEFAELCEHQLTHHFRVARKIALSDGASLALVTPETTATSSTEQFALANFVKTTLHAFTATIGVESERTAQRILINQVDLTRRARAEEPRDPRERQQELERFIEAVLLVTAPLPPEADTRYAGRIHRGRAITV

SEQ ID NO: 108 - protein, 1221 aa, Oscillochloris trichoides
MFMTRLNDKIALITGGAGTIGEVITRRYLEEGATVVMAGRNRDKLDRYRERLITEFHALPERVMVVRMDGSSNAEVRMGIAAVVAHFGRIDILVNNAGSAGARQRLPAIPLLRSELQADETETLADSIGNLIGITWNLIRAAAPFMPAGSSVINISTIFARTDYYGRIPYVVPKAALHALTLAAATELGERGIRVNQINPGPIDSDRIRTVFRRMDELKGVPEQSTADGFFQMMRLRRPNAEGDLVKGFPKTLDVANVAVFLGSAESAALSGETLDVTHGMAVPTESRTTLTSRPGLRAVDGSGHTTLICVGDQIEEAAALTGVLRACGAEVVIGFRSRAAIARFDHLIERGRHLPSQEHVAPVLLYLNPTEPESIDQALRWMATNLDLPTSVIILPAQRQPLPPSVVRASDEEVAYFLRDELSGMIVLASRLARFWQQATLAPGNAPIQPRVLFMTNPDDGQGNLYAEILRAGVEQLCRVWRHESQLDYTRLAQMDAHPPHIRPVWANQLVRFANNEQENLEYCCAWVAKILLSERTIEELNLYLPRQIGSTTGSRQPSFGWAENLIGLHLGKTALITGGSAGIGSQIARLLALSGARVMLCARDERKLIQMRDMIIAELTEVGYNQVESRVQICAGCDVGEEEQLEIAVQRTLDLFGHVDYLINNAGIAGAEEMVLDLPLEAWQRTLRTNLISNYSLIRKLAPQMKSRGSGYILNVSSYFGGEKYAAIPYPNRADYAVSKAGQRALGEALARLLGPEVQINAMAPGPVEGERLRGSGDRPGLFLRRGRLILENKRLNDLHATLIAAERETQVGMRDLLARLLHNDVCALIDDPAAPTHLRALAERIWEQSDPNSYARAFFMNANIATKLLARLFNADQIDAQTFHTSQPNLPPEPFFARTQIDREARRVRDGVMSMLYLQRMPTEFDVALATVYYLNDRSVSGETFHPSGGLRHERTPTGAELYGSPAPQRLASLAGSTVYLIGESMAAHLEALARAYIERYAATRVVLICATPAGVERFSHHLADHLASGALAILSAEEGIEAALSEALRRFGPPGPVVSTPFQPLPSQPLIGRNDSDWSTVLDVAGFSAMCEQQLTHHFRVTRKLSLVAGVSLVLVTPETDSHSSTEQFALANFVKTTLHAFTATVGVECERTAHRILVNQVDLGRQARAEEPRSPAEQAQEMERFIDAIMLTTAPIPAEEDNRYTGRIYRGRAITV

SEQ ID NO: 109 - protein, 1220 aa, Chloroflexus aurantiacus
MSGTGRLAGKIALITGGAGNIGSELTRRFLAEGATVIISGRNRAKLTALAERMQAEAGVPAKRIDLEVMDGSDPVAVRAGIEAIVARHGQIDILVNNAGSAGAQRRLAEIPLTEAELGPGAEETLHASIANLLGMGWHLMRIAAPHMPVGSAVINVSTIFSRAEYYGRIPYVTPKAALNALSQLAARELGARGIRVNTIFPGPIESDRIRTVFQRMDQLKGRPEGDTAHHFLNTMRLCRANDQGALERRFPSVGDVADAAVFLASAESAALSGETIEVTHGMELPACSETSLLARTDLRTIDASGRTTLICAGDQIEEVMALTGMLRTCGSEVIIGFRSAAALAQFEQAVNESRRLAGADFTPPIALPLDPRDPATIDAVFDWGAGENTGGIHAAVILPATSHEPAPCVIEVDDERVLNFLADEITGTIVIASRLARYWQSQRLTPGARARGPRVIFLSNGADQNGNVYGRIQSAAIGQLIRVWRHEAELDYQRASAAGDHVLPPVWANQIVRFANRSLEGLEFACAWTAQLLHSQRHINEITLNIPANISATTGARSASVGWAESLIGLHLGKVALITGGSAGIGGQIGRLLALSGARVMLAARDRHKLEQMQAMIQSELAEVGYTDVEDRVHIAPGCDVSSEAQLADLVERTLSAFGTVDYLINNAGIAGVEEMVIDMPVEGWRHTLFANLISNYSLMRKLAPLMKKQGSGYILNVSSYFGGEKDAAIPYPNRADYAVSKAGQRAMAEVFARFLGPEIQINAIAPGPVEGDRLRGTGERPGLFARRARLILENKRLNELHAALIAAARTDERSMHELVELLLPNDVAALEQNPAAPTALRELARRFRSEGDPAASSSSALLNRSIAAKLLARLHNGGYVLPADIFANLPNPPDPFFTRAQIDREARKVRDGIMGMLYLQRMPTEFDVAMATVYYLADRNVSGETFHPSGGLRYERTPTGGELFGLPSPERLAELVGSTVYLIGEHLTEHLNLLARAYLERYGARQVVMIVETETGAETMRRLLHDHVEAGRLMTIVAGDQIEAAIDQAITRYGRPGPVVCTPFRPLPTVPLVGRKDSDWSTVLSEAEFAELCEHQLTHHFRVARKIALSDGASLALVTPETTATSTTEQFALANFIKTTLHAFTATIGVESERTAQRILINQVDLTRRARAEEPRDPHERQQELERFIEAVLLVTAPLPPEADTRYAGRIHRGRAITV

SEQ ID NO: 110 - DNA, 24 bp, Artificial Sequence
  Description of Artificial Sequence: Synthetic oligonucleotide
gcgtagtaaaggaggtaacatatg

SEQ ID NO: 111 - DNA, 29 bp, Artificial Sequence
  Description of Artificial Sequence: Synthetic oligonucleotide
caatttatttaaggaggactcttaagatg

SEQ ID NO: 112 - DNA, 37 bp, Artificial Sequence
  Description of Artificial Sequence: Synthetic oligonucleotide
gaaatttcataccacaggcgaaggaggaaaaaccatg

SEQ ID NO: 113 - DNA, 22 bp, Artificial Sequence
  Description of Artificial Sequence: Synthetic oligonucleotide
ggaagaacaaggggtgtacatg

SEQ ID NO: 114 - DNA, 32 bp, Artificial Sequence
  Description of Artificial Sequence: Synthetic oligonucleotide
ggaagaattaagggggacaagggggaataatg

SEQ ID NO: 115 - DNA, 34 bp, Artificial Sequence
  Description of Artificial Sequence: Synthetic oligonucleotide
gcgtagtagccgggtgataaggagccgtaacatg

SEQ ID NO: 116 - DNA, 34 bp, Artificial Sequence
  Description of Artificial Sequence: Synthetic oligonucleotide
gcgtagtagctgatataaaaggaggtaacggatg

SEQ ID NO: 117 - DNA, 37 bp, Artificial Sequence
  Description of Artificial Sequence: Synthetic oligonucleotide
caatttatttttgttcacccaaggagtattgctaatg

SEQ ID NO: 118 - DNA, 37 bp, Artificial Sequence
  Description of Artificial Sequence: Synthetic oligonucleotide
caatttatttaccgaaataaaaggagggatgcgaatg

SEQ ID NO: 119 - DNA, 31 bp, Artificial Sequence
  Description of Artificial Sequence: Synthetic oligonucleotide
tcttcccacaacactggcggactccatcatg

SEQ ID NO: 120 - protein, 25 aa, Artificial Sequence
  Description of Artificial Sequence: Synthetic peptide
LSGGGGSGGGGSGGGGSGGGGSAAA

(19) United States
(12) Patent Application Publication
(10) Pub. No.: US 2015/0064754 A1
(43) Pub. Date: Mar. 5, 2015

(54) BIOPRODUCTION OF CHEMICALS

(71) Applicant: OPX Biotechnologies, Inc., Boulder, CO (US)

(72) Inventors: Hans LIAO, Superior, CO (US); Christopher Patrick MERCOGLIANO, Minneapolis, MN (US); Travis Robert WOLTER, Denver, CO (US); Michael Tai Man LOUIE, Broomfield, CO (US); Wendy Kathleen RIBBLE, Arvada, CO (US); Tanya LIPSCOMB, Boulder, CO (US); Eileen Colie SPINDLER, Lafayette, CO (US); Michael D LYNCH, Durham, NC (US)

(21) Appl. No.: 14/215,626

(22) Filed: Mar. 17, 2014

Related U.S. Application Data
(60) Provisional application No. 61/852,387, filed on Mar. 15, 2013.
(60) Provisional application No. 61/791,743, filed on Mar. 15, 2013.

(51) Int. Cl. C12P 7/42; C07K 14/245; C12N 9/00; C12N 9/02; C12N 9/04

(52) U.S. Cl. CPC C12P 7/42; C12N 9/0008; C12N 9/0006; C12N 9/93; C07K 14/245; C12Y 102/01075; C12Y 101/01059; C12Y 604/01002
USPC 435/146; 435/252.33

(57) ABSTRACT

The present invention provides various combinations of genetic modifications to a transformed host cell that provide increase conversion of carbon to a chemical product. The present invention also provides methods of fermentation and methods of making various chemical products.

This application claims priority to U.S. Provisional Patent Application No. 61/852,387, filed on Mar. 15, 2013; and U.S. Provisional Patent Application No. 61/791,743, filed on Mar. 15, 2013; which are herein incorporated by reference in their entirety.

### BACKGROUND OF THE INVENTION

There is a need for alternative production methods of industrial chemicals used for various consumer products and fuels that are currently made from petroleum. One alternative method is the use of engineered microorganisms to produce industrial chemicals. Currently, in the field of bioproduced chemicals there is a need to improve microbial enzyme performance, enhanced production rate in order to reach the goal of becoming an at-cost replacement basis for petro-based chemicals.

A common challenge faced in field of bio-produced chemicals in microorganisms is that any one modification to a host cell may require coordination with other modifications in order to successfully enhance chemical bioproduction.

The current invention provides methods, systems of fermentation, genetically modified microorganisms, modified enhanced enzymes for chemical production, all of which may be used in various combinations to increase chemical production of a desired chemical product.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY OF THE INVENTION

The present invention relates to genetically modified organisms capable of producing an industrial chemical product of interest, wherein the genetic modification includes introduction of nucleic acid sequences coding for polynucleotides encoding one or more of the following: (1) an acetyl-CoA carboxylase gene with one or more of its subunits fused together in the genetic structure of the organism; (2) an acetyl-CoA carboxylase gene having a predefined stoichiometric ratio of each of the four ACCase subunits relative to one another; (3) a monofunctional malonyl-CoA reductase gene capable of catalyzing the conversion of malonyl-CoA to malonate semialdehyde and one or more genes encoding one or more of the following enzymes: ydfG, mmsB, NDSD, rutE, and nemA; (4) a monofunctional malonyl-CoA reductase gene capable of catalyzing the conversion of malonyl-CoA to malonate semialdehyde and one or more genes encoding one or more enzymes capable of converting malonate semialdehyde keto form to 3-HP, and one or more genes encoding one or more enzymes capable of converting either the malonate semialdehyde enol form or the malonate semialdehyde hydrated form to 3-HP; (5) a monofunctional malonyl-CoA reductase enzyme fused to a dehydrogenase enzyme that is either: (a) primarily not NADPH-dependent, (b) primarily NADH-dependent, (c) primarily flavin-dependent, (d) less susceptible to 3-HP inhibition at high concentration, and/or (e) catalyzes a reaction pathway to 3-HP that is substantially irreversible; (6) a monofunctional malonyl-CoA reductase enzyme fused to one or more malonate semialdehyde dehydrogenase enzymes; (7) a malonyl-CoA reductase gene that is mutated to enhance its activity at lower temperatures; (8) salt-tolerant enzymes; (9) a gene that facilitates the exportation of a chemical product of interest or the export of an inhibitory chemical from within the cell to the extracellular media; and/or (10) a gene that facilitates the importation from the extracellular media to within the cell of a reactant, precursor, and/or metabolite used in the organism's production pathway for producing a chemical product of interest.

The present invention further relates to methods of producing a chemical product using the genetically modified organisms of the invention. The present invention further includes products made from these methods. In accordance with certain embodiments that product is acetyl-CoA, malonyl-CoA, malonate semialdehyde, 3-hydroxypropionic acid (3-HP), acrylic acid, 1,3 propanediol, malonic acid, ethyl 3-HP, propiolactone, acrylonitrile, acrylamide, methyl acrylate, a polymer including super absorbent polymers and polyacrylic acid, or a consumer product.

The present invention further relates to a method of producing a chemical product from a renewable carbon source through a bioproduction process that comprises a controlled multi-phase production process wherein the initiation and/or completion of one or more phases of the production process is controlled by genetic modifications to the organism producing the chemical product and/or is controlled by changes made to the cell environment. In accordance with this aspect of the invention, the bioproduction process may include two or more of the following phases: (1) growth phase; (2) induction phase; and (3) production phase. The present invention further includes products made from these methods.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 Depicts some embodiments of the metabolic pathways to produce 3-hydroxypropionic acid.

FIG. 2 Depicts some embodiments of the of various equilibrium states in the malonate semialdehyde to 3-HP reaction in a cell environment.

FIG. 3 Depicts some embodiments of the reaction catalyzed by acetyl-CoA carboxylase (ACCase)

FIG. 4 Shows the inhibition of ACCase enzyme activity by high salt concentration

FIG. 5 Depicts some embodiments of the fusion ACCase subunit gene constructs overexpressed in E. coli.

FIG. 6 Show improved production of 3-HP by genetically modified organism with DA fusion ACCase

FIG. 7 Shows improved production of 3-HP by genetically modified organism with overexpression of rhtA exporter.

FIG. 8 Shows various embodiments of the genetic modules used for optimizing expression in host cells.

FIG. 9 Shows various chemical products that can made from various embodiments of the invention.

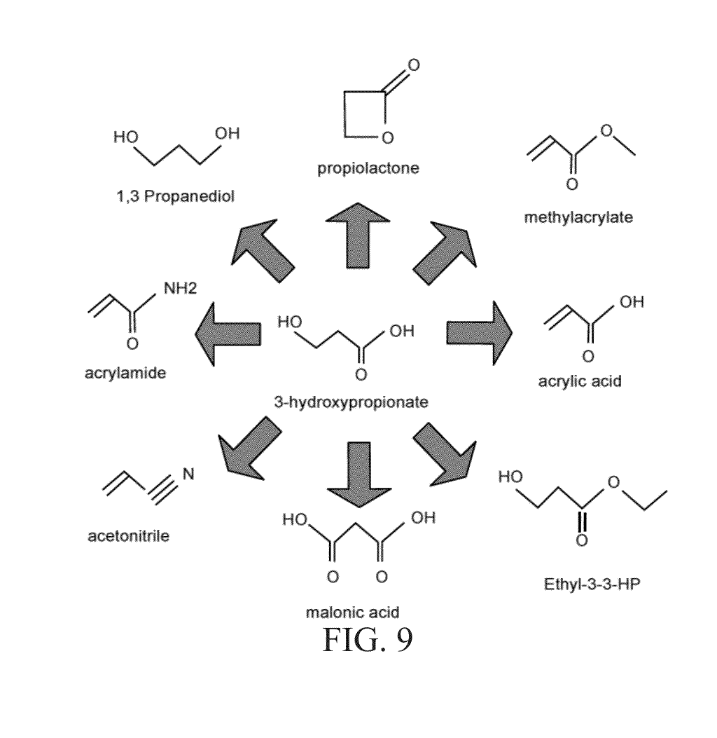

Table 1 Lists the accession numbers for genes encoding ACCase subunits from Halomonas elongate.

Table 2 Depicts some embodiments of the RBS sequences used to enhance expression of H. elongate ACCase subunits.

Table 3 Shows the improvement in 3-HP production by RBS-optimized expression of H. elongata ACCase subunits.

Table 4 Shows some embodiments of the ACCase subunit fusions that increase and ACCase enzyme complex activity.

Table 5 Shows some of the genetic modifications of a host cell for increase chemical production.

### DETAILED DESCRIPTION OF THE INVENTION

### Definitions

The term “homology” refers to the optimal alignment of sequences (either nucleotides or amino acids), which may be conducted by computerized implementations of algorithms. “Homology”, with regard to polynucleotides, for example, may be determined by analysis with BLASTN version 2.0 using the default parameters. “Homology”, with respect to polypeptides (i.e., amino acids), may be determined using a program, such as BLASTP version 2.2.2 with the default parameters, which aligns the polypeptide or fragments (and can also align nucleotide fragments) being compared and determines the extent of amino acid identity or similarity between them. It will be appreciated that amino acid “homology” includes conservative substitutions, i.e. those that substitute a given amino acid in a polypeptide by another amino acid of similar characteristics. Typically seen as conservative substitutions are the following replacements: replacements of an aliphatic amino acid such as Ala, Val, Leu and He with another aliphatic amino acid; replacement of a Ser with a Thr or vice versa; replacement of an acidic residue such as Asp or Glu with another acidic residue; replacement of a residue bearing an amide group, such as Asn or Gln, with another residue bearing an amide group; exchange of a basic residue such as Lys or Arg with another basic residue; and replacement of an aromatic residue such as Phe or Tyr with another aromatic residue. For example, homologs can have at least 99%, 98%, 97%, 96%, 95%, 94%, 93%, 92%, 91%, 90%, 89%, 88%, 87%, 86%, 85%, 84%, 83%, 82%, 81% or 80% overall amino acid or nucleotide identity to the gene or proteins of the invention; or can have 99%, 98%, 97%, 96%, 95%, 94%, 93%, 92%, 91%, 90%, 89%, 88%, 87%, 86%, 85%, 84%, 83%, 82%, 81% or 80% amino acid or nucleotide to the essential protein functional domains of the gene or proteins of the invention; or at least 99%, 98%, 97%, 96%, 95%, 94%, 93%, 92%, 91%, 90%, 89%, 88%, 87%, 86%, 85%, 84%, 83%, 82%, 81% or 80% overall amino acid or nucleotide to the essential binding amino acids within an essential functional domain of the gene or proteins of the invention.

The above descriptions and methods for sequence homology are intended to be exemplary and it is recognized that this concept is well-understood in the art. Further, it is appreciated that nucleic acid sequences may be varied and still provide a functional enzyme, and such variations are within the scope of the present invention. The term “enzyme homolog” can also mean a functional variant.

The term “Functional homolog” means a polypeptide that is determined to possess an enzymatic activity and specificity of an enzyme of interest but which has an amino acid sequence different from such enzyme of interest. A corresponding “homolog nucleic acid sequence” may be constructed that is determined to encode such an identified enzymatic functional variant.

The term “3-HP” means 3-hydroxypropionic acid.

The term “heterologous DNA,” “heterologous nucleic acid sequence,” and the like as used herein refers to a nucleic acid sequence wherein at least one of the following is true: (a) the sequence of nucleic acids is foreign to (i.e., not naturally found in) a given host microorganism; (b) the sequence may be naturally found in a given host microorganism, but in an unnatural (e.g., greater than expected) amount; or (c) the sequence of nucleic acids comprises two or more subsequences that are not found in the same relationship to each other in nature. For example, regarding instance (c), a heterologous nucleic acid sequence that is recombinantly produced will have two or more sequences from unrelated genes arranged to make a new functional nucleic acid. Embodiments of the present invention may result from introduction of an expression vector into a host microorganism, wherein the expression vector contains a nucleic acid sequence coding for an enzyme that is, or is not, normally found in a host microorganism. With reference to the host microorganism's genome prior to the introduction of the heterologous nucleic acid sequence, then, the nucleic acid sequence that codes for the enzyme is heterologous (whether or not the heterologous nucleic acid sequence is introduced into that genome). The term “heterologous” is intended to include the term “exogenous” as the latter term is generally used in the art as well as “endogenous”.

As used in the specification and the appended claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “microorganism” includes a single microorganism as well as a plurality of microorganisms; and the like.

I. Introduction

The present invention relates to various genetically modified microorganisms, methods for making the same, and use of the same in making industrial products. Any and all of the microorganisms herein may include a combination of genetic alterations as described herein. The present invention contemplates, for example, a genetically modified microorganism having one or more of the following genetic modifications (i) an alteration that affects the stoichiometric ratio, expression or production of one or more ACCase enzyme genes (ii) a recombinant ACCase gene having at least 80% sequence homology to an ACCase gene from a salt tolerant organism (iii) a genetic alteration in one or more non-ACCase genes (iv) one or more genetic alterations that encodes for one or more exporters capable of exporting 3-HP out of a cell (v) new hybrid molecules or co-expressed of a mono-functional malonyl-CoA reductase enzyme with various 3-HP dehydrogenase proteins that: (a) exhibit less inhibition by high 3-HP concentrations (b) that is less reversible or irreversible (c) enzymes that utilized NADH (d) enzymes that utilized flavin (vi) one or more genetic alterations that can be used to switch the carbon in the standard metabolic pathways of the cells to a pathway engineered to produce a chemical. More details about each of the above modifications and how the modification are used together to increase chemical production in a host cell is described below.

The present invention also relates to methods of fermentation. The genetically modified microorganisms are cultured under conditions that optimized a host cell for increase chemical production. The bio-production process may include two or more of the following phases of fermentation: (1) growth phase where the culture organism replicates itself and the carbon intermediate product is built up; (2) the induction phase, where the expression of key enzymes critical to the chemical production is induced and the enzymes accumulate within the organism to carry out the engineered pathway reactions required to further produce the chemical product (3) production phase is where the organism expresses proteins that provide for continuously production the desired chemical product. The above phases are further controlled by (1) addition and amount of the initiating reactant added to the reaction vessel (2) key enzymes engineered into the organism using promoters that are sensitive to (e.g., activated by) the depletion of the initiating reactant. Addition details about the fermentation process of the invention are disclosed below.

II. Acetyl-CoA Carboxylase

Malonyl-CoA Flux

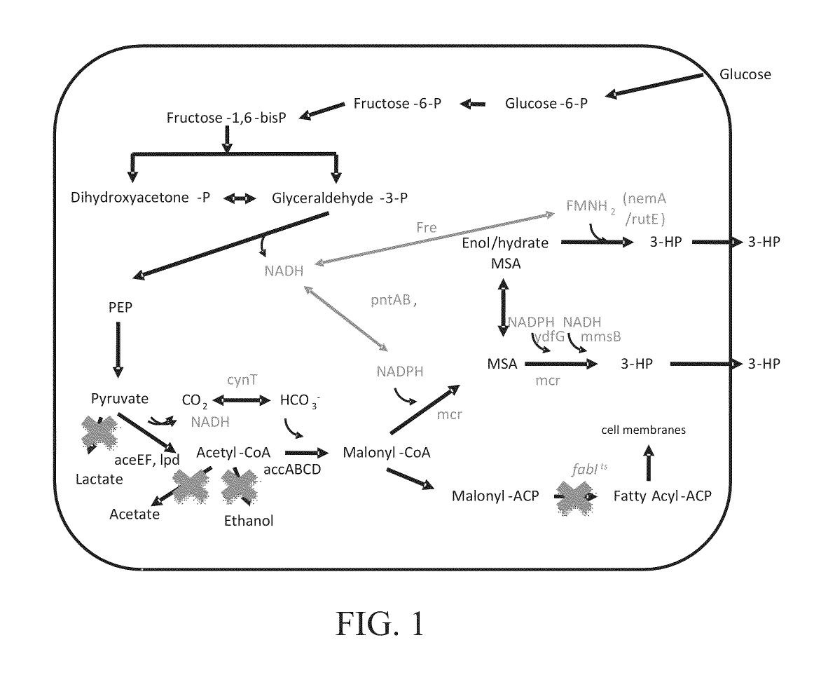

One of the steps in the biosynthesis of 3-HP involves the reaction catalyzed by acetyl-CoA carboxylase (ACCase) enzyme. ACCase is a primary control point in the 3-HP pathway shown in FIG. 1 (previously described in) for the converting acetyl-CoA to malonyl-CoA and hence to malonate semialdehyde and 3-HP. The present invention contemplates the use of genetic modifications that increase activity of ACCase complex enzymes to thereby increase 3-HP production in a host cell.

Fused Subunits

The acetyl-CoA carboxylase complex (ACCase) is a multi-subunit protein. Prokaryotes and plants have multi-subunit ACCs composed of several polypeptides encoded by distinct genes. However, humans and most other eukaryotes, such as yeast, have evolved an ACC with CT and BC catalytic domains and biotin carboxyl carrier domains on a single polypeptide. The biotin carboxylase (BC) activity, biotin carboxyl carrier protein (BCCP), and carboxyl transferase (CT) activity are each contained on a different subunit. In E. coli the ACCase complex is derived from multi polypeptide transcribed by distinct, separable protein components known as accA, accB, accC, and accD.

Acetyl-CoA carboxylase is a biotin-dependent enzyme that catalyzes the irreversible carboxylation of acetyl-CoA to produce malonyl-CoA through its two catalytic activities, biotin carboxylase (BC) and carboxyltransferase (CT). The first reaction is carried out by BC and involves the ATP-dependent carboxylation of biotin with bicarbonate. The carboxyl group is transferred from biotin to acetyl-CoA to form malonyl-CoA in the second reaction, which is catalyzed by CT. The main function of ACCase complex in the cell is to provide the malonyl-CoA substrate for the biosynthesis of fatty acids.

The conversion of acetyl-CoA to malonyl-CoA is an important step in the bioconversion of a renewable carbon source (such as, for example, sugar or natural gas) to a useful industrial chemical (such as, for example, 3-hydroxypropionic acid (3-HP)). In certain organisms, such as E. coli or yeast, the native ACCase expression from the chromosome alone is insufficient to enable the organism to produce chemicals such as 3-HP at a rate to support a commercial scale operation. Overexpression of the ACCase complex has been shown to provide some advantage [U.S. Ser. No. 12/891,760 U.S. Ser. No. 12/891,790 U.S. Ser. No. 13/055,138].

Applicants have discovered that the introduction of an acetyl-CoA carboxylase gene with one or more of its subunits fused is beneficial to the production of a chemical product in a host cell. In certain aspects of the invention, fusion is the two gene products produced from a single polypeptide controlled by a single promoter, will further enhance an organism's bioproduction of an industrial chemical. In certain aspects of the invention, fusion is the two gene products produced by at least one promoter, will further enhance an organism's bioproduction of an industrial chemical. In certain aspects of the invention, fusion is the two gene products produced from a single polypeptide controlled by at least one inducible promoter, will further enhance an organism's bioproduction of an industrial chemical. Keeping components of the ACCase complex fused together in the genetic structure of an organism can be advantageous because it enhances the stability of the non-native ACCase genetic modification and it facilitates equimolar expression of the fused acc subunits.

In particular, the subunit-fused ACCase may be an accA-accB, accA-accC, accA-accD, accB-accC, accB-accD, accC-accD, accA-accB-accC, accA-accB-accD, accA-accC-accD, accB-accC-accD or accA-accB-accC-accD fused subunit that have having at least 80% sequence homology to E. coli accA, accB, accC and accD or is a functional homolog thereof. In addition, the organism may include any combination of these fused subunits, or any combination of these fused subunits together with one or more of the four non-fused subunits. When such combinations are used, the subunits (fused and non-fused) may be expressed on the same plasmid or on different plasmids or on the chromosome of the organism.

In accordance with a preferred embodiment, an accA-accD fused subunit is introduced into an organism either alone or in combination with the accB-accC fused subunit, the accB gene, and/or the accC gene. In accordance with a preferred embodiment, the organism is a bacteria, and preferably E. coli or Cupriavidus necator.

Composition Stoichiometry

Composition stoichiometry is the quantitative relationships among elements that comprise a compound. A stoichiometric ratio of a reagent is the optimum amount or ratio where, assuming that the reaction proceeds to completion. Although stoichiometric terms are traditionally reserved for chemical compounds, theses theoretical consideration of stoichiometry are relevant when considering the optimal function of heterologous multi-subunit protein in a host cell.

In accordance with another aspect of the invention, the stoichiometric ratio of each of the four ACCase subunits relative to one another is important, and each such ratio can be between 0 and about 10, and preferably between about 0.5 to about 2 or about 7 to about 9. In accordance with a preferred embodiment the ratios for the protein subunits are accA:accB:accC:accD are 1:2:1:1. In accordance with a preferred embodiment, an organism is genetically modified to include an accA-accD fused subunit, an accB non-fused subunit, and an accC non-fused subunit, with the molar ratios of the accDA fusion:accB:accC being about 1:2:1, which is close to the optimum for enzymatic activity.

In certain embodiments where an organism is engineered to make 3-HP, in order to get optimal function in a host cell of a heterologous ACCase enzyme complex it is important to engineer the stoichiometry of these subunits in such a way that provides maximal production of 3-HP such that the subunit can make a more stable enzyme complex when overexpressed in the cell.

In certain aspects the invention provides for the controlled expression of the natural accA, accB, accC, and accD subunits of E. coli or having at least 80% sequence homology to E. coli accA, accB, accC and accD. In certain aspects the invention provides for the inducible expression of the natural accA, accB, accC, and accD subunits of E. coli or having at least 80% sequence homology to E. coli accA, accB, accC, and accD. In certain aspects the invention provides for the low, medium, high and/or inducible expression of the natural accA, accB, accC, and accD subunits of E. coli or having at least 80% sequence homology to E. coli accA, accB, accC and accD.

In certain aspects the invention provides for the expression of the natural accC and accD subunits of E. coli or having at least 80% sequence homology to E. coli accA, accB, accC and accD in low, medium, high or inducible expression. In certain aspects the invention provides for the expression of the natural accB and accA subunits of E. coli or having at least 80% sequence homology to E. coli accA, accB, accC, and accD in low, medium, high or inducible expression. In certain aspects the invention provides for the expression of the natural accC and accD subunits with the accA subunit of E. coli or having at least 80% sequence homology to E. coli accA, accB, accC, and accD in low, medium, high or inducible expression. In certain aspects the invention provides for the expression of the natural accC and accD subunits with the accB subunit of E. coli or having at least 80% sequence homology to E. coli accA, accB, accC, and accD in low, medium, high or inducible expression.

In certain aspects the invention provides for the expression of a fusion of two, three, or all of the four ACCase subunits in one polypeptide in low, medium, high or inducible expression. Such fusion may include any of the following combinations of the ACCase subunits: accA-accB, accA-accC, accA-accD, accB-accC, accB-accD, accC-accD, accA-accB-accC, accA-accB-accD, accA-accC-accD, accB-accC-accD, and accA-accB-accC-accD have having at least 80% sequence homology to E. coli accA, accB, accC and accD or is a functional homolog thereof.

In certain aspects the invention provides for ACC complex in the stoichiometry of these subunits of the accCB and accDA in a 1:1, 1:2, 1:3, 1:4, 1:5, 1:6, 1:7, 1:8, 2:1, 2:3, 2:4, 2:5, 2:6, 2:7, 2:8, 3:1, 3:3, 3:4, 3:5, 3:6, 3:7, 3:8, 3:1, 3:3, 3:4, 3:5, 3:6, 3:7, 3:8, 4:1, 4:3, 4:4, 4:5, 4:6, 4:7, 4:8, 5:1, 5:3, 5:4, 5:5, 5:6, 5:7, 5:8, 6:1, 6:3, 6:4, 6:5, 6:6, 6:7, 6:8, 7:1, 7:3, 7:4, 7:5, 7:6, 7:7, 7:8, 8:1, 8:3, 8:4, 8:5, 8:6, 8:7, or 8:8 in low, medium, high or inducible expression. In certain aspects the invention provides for ACC complex in the stoichiometry of these subunits of the accDA and accCB in a 1:1, 1:2, 1:3, 1:4, 1:5, 1:6, 1:7, 1:8, 2:1, 2:3, 2:4, 2:5, 2:6, 2:7, 2:8, 3:1, 3:3, 3:4, 3:5, 3:6, 3:7, 3:8, 3:1, 3:3, 3:4, 3:5, 3:6, 3:7, 3:8, 4:1, 4:3, 4:4, 4:5, 4:6, 4:7, 4:8, 5:1, 5:3, 5:4, 5:5, 5:6, 5:7, 5:8, 6:1, 6:3, 6:4, 6:5, 6:6, 6:7, 6:8, 7:1, 7:3, 7:4, 7:5, 7:6, 7:7, 7:8, 8:1, 8:3, 8:4, 8:5, 8:6, 8:7, or 8:8 in low, medium, high or inducible expression.

In certain aspects the invention provides for the stoichiometry of the accD-A subunits in a 1:1, 1:2, 1:3, 1:4, 1:5, 1:6, 1:7, 1:8, 2:1, 2:3, 2:4, 2:5, 2:6, 2:7, 2:8, 3:1, 3:3, 3:4, 3:5, 3:6, 3:7, 3:8, 3:1, 3:3, 3:4, 3:5, 3:6, 3:7, 3:8, 4:1, 4:3, 4:4, 4:5, 4:6, 4:7, 4:8, 5:1, 5:3, 5:4, 5:5, 5:6, 5:7, 5:8, 6:1, 6:3, 6:4, 6:5, 6:6, 6:7, 6:8, 7:1, 7:3, 7:4, 7:5, 7:6, 7:7, 7:8, 8:1, 8:3, 8:4, 8:5, 8:6, 8:7, or 8:8 in low, medium, high or inducible expression. In certain aspects the invention provides for the stoichiometry of the accC-B subunits in a 1:1, 1:2, 1:3, 1:4, 1:5, 1:6, 1:7, 1:8, 2:1, 2:3, 2:4, 2:5, 2:6, 2:7, 2:8, 3:1, 3:3, 3:4, 3:5, 3:6, 3:7, 3:8, 3:1, 3:3, 3:4, 3:5, 3:6, 3:7, 3:8, 4:1, 4:3, 4:4, 4:5, 4:6, 4:7, 4:8, 5:1, 5:3, 5:4, 5:5, 5:6, 5:7, 5:8, 6:1, 6:3, 6:4, 6:5, 6:6, 6:7, 6:8, 7:1, 7:3, 7:4, 7:5, 7:6, 7:7, 7:8, 8:1, 8:3, 8:4, 8:5, 8:6, 8:7, or 8:8 in low, medium, high or inducible expression. In certain aspects the invention provides for the stoichiometry of the accC-A subunits in a 1:1, 1:2, 1:3, 1:4, 1:5, 1:6, 1:7, 1:8, 2:1, 2:3, 2:4, 2:5, 2:6, 2:7, 2:8, 3:1, 3:3, 3:4, 3:5, 3:6, 3:7, 3:8, 3:1, 3:3, 3:4, 3:5, 3:6, 3:7, 3:8, 4:1, 4:3, 4:4, 4:5, 4:6, 4:7, 4:8, 5:1, 5:3, 5:4, 5:5, 5:6, 5:7, 5:8, 6:1, 6:3, 6:4, 6:5, 6:6, 6:7, 6:8, 7:1, 7:3, 7:4, 7:5, 7:6, 7:7, 7:8, 8:1, 8:3, 8:4, 8:5, 8:6, 8:7, or 8:8 in low, medium, high or inducible expression. In certain aspects the invention provides for the stoichiometry of the accC-B subunits in a 1:1, 1:2, 1:3, 1:4, 1:5, 1:6, 1:7, 1:8, 2:1, 2:3, 2:4, 2:5, 2:6, 2:7, 2:8, 3:1, 3:3, 3:4, 3:5, 3:6, 3:7, 3:8, 3:1, 3:3, 3:4, 3:5, 3:6, 3:7, 3:8, 4:1, 4:3, 4:4, 4:5, 4:6, 4:7, 4:8, 5:1, 5:3, 5:4, 5:5, 5:6, 5:7, 5:8, 6:1, 6:3, 6:4, 6:5, 6:6, 6:7, 6:8, 7:1, 7:3, 7:4, 7:5, 7:6, 7:7, 7:8, 8:1, 8:3, 8:4, 8:5, 8:6, 8:7, or 8:8 in low, medium, high or inducible expression.

III. Conversion of Malonyl-CoA to Malonate Semialdehyde

One of the steps in the biosynthesis of 3-HP involves the conversion of malonyl-CoA (MCA) to malonate semialdehyde (MSA) and the conversion of malonate semialdehyde (MSA) to 3-HP (WO2011/038364). In accordance with another aspect of the present invention, the present invention contemplates the use of novel enzymes and/or combinations of enzymes to catalyze the reaction in a microorganism from MCA to MSA, which results in enhanced cellular bioproduction of 3-HP in the host cell.

In certain aspects the invention provides novel enzyme compositions or co-expression of a combinations of enzyme compositions to catalyze the conversion of malonyl-CoA to 3-HP. A general overview of the enzymes and the relevant reaction pathways methods are shown in FIG. 1.

In accordance with this aspect of the invention, malonyl-CoA is converted to malonate semialdehyde by a malonyl-CoA reductase and malonate semialdehyde is converted to 3-HP through either or both of two alternative pathways.

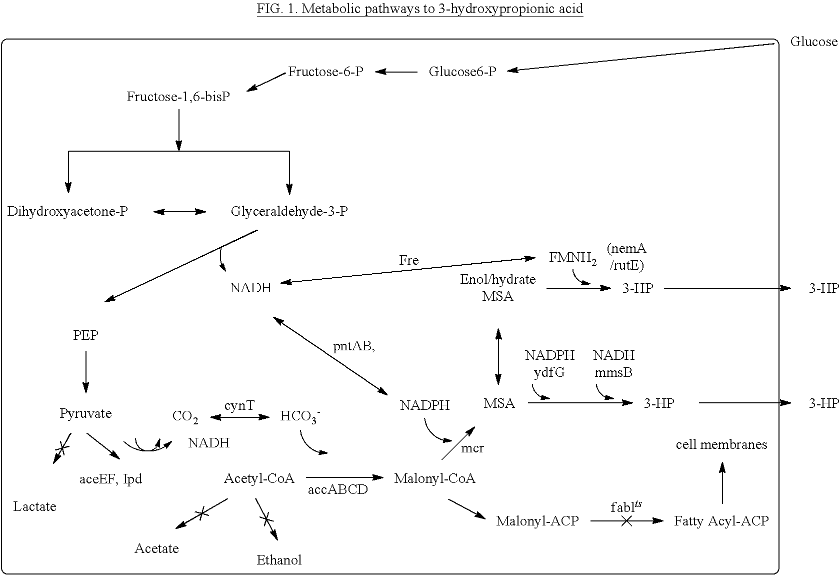

In accordance with one aspect of the invention, malonyl-CoA is converted to malonate semialdehyde by a monofunctional malonyl-CoA reductase that catalyzes the malonyl-CoA conversion, but does not catalyze the malonate semialdehyde conversion.

In one embodiment, the microorganism herein comprise a genetic modification that include the monofunctional malonyl-CoA reductase may be derived from Sulfolobus tokodaii (stMCR) (SEQ ID NO. 15 nucleic acid, SEQ ID NO. 16 protein sequence) or a functional homolog of stMCR or a homolog with at least 80% identity.

In some embodiments, the microorganism herein comprise a genetic modification that include the bi-functional malonyl-CoA reductase comprised of two protein fragments with one fragment having malonyl-CoA reductase activity and the other fragment having malonate semialdehyde dehydrogenase activity may be derived from Chloroflexus aurantiacus (caMCR).

### MCR-Dehydrogenase Enzymes for Conversion of 3-HP Ions

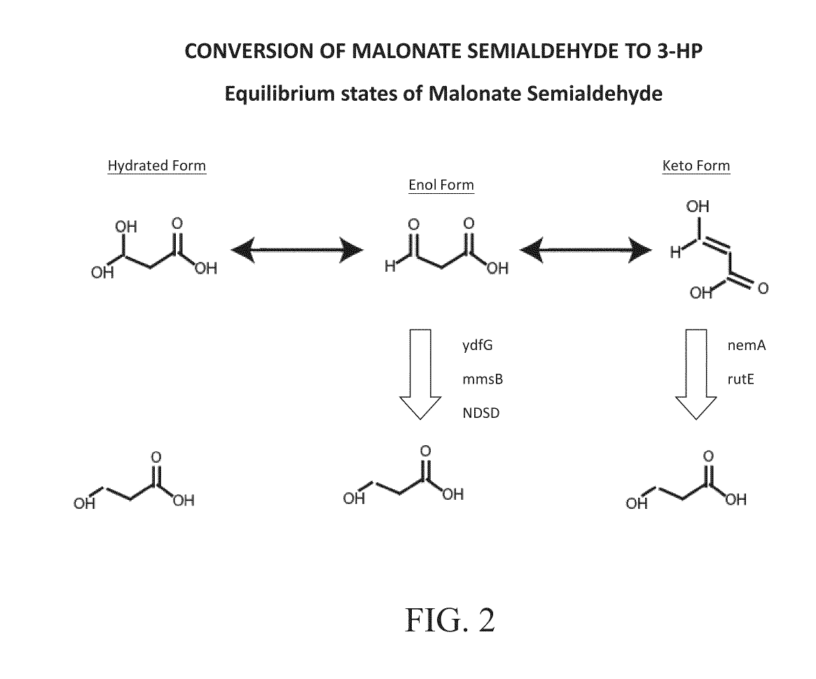

Following the conversion of the malonyl-CoA to malonate semialdehyde, the malonate semialdehyde is converted to 3-HP through either or both of two alternative pathways. Malonate semialdehyde may exist in at least three states; the keto form, the enol form, and hydrate form, as shown in FIG. 2. Malonate semialdehyde in the enol form, which will stabilize this form when compared to other aldehydes where the enol form is highly unfavored in the equilibrium among the three forms.

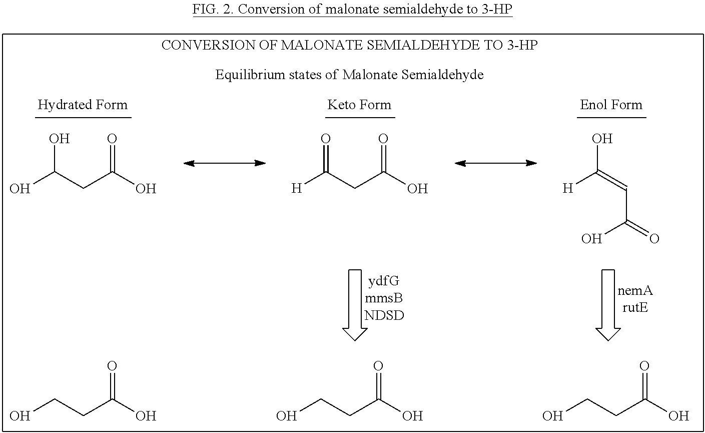 [SMILES: O=C(O)CC(O)O ; O=CCC(=O)O ; O=C(O)/C=C/O ; O=C(O)CCO ; O=C(O)CCO ; O=C(O)CCO]

The malonate semialdehyde keto form is converted to 3-HP utilizing a 3-hydroxy acid dehydrogenase enzyme (ydfG SEQ ID NO. 21 nucleic acid, SEQ ID NO. 22 protein), a 3-hydroxyisobutyrate dehydrogenase enzyme (Pseudomonas aeruginosa mmsB, SEQ ID No 23 nucleic acid, SEQ ID NO. 24 protein), and/or NAD+-dependent serine dehydrogenase (Pseudomonas NDSD, SEQ ID NO. 25 nucleic acid, SEQ ID NO. 26 protein). In accordance with a preferred embodiment, Pseudomonas mmsB, Pseudomonas NDSD, and E. coli ydfG are used. The gene, ydfG from E. coli is largely NADPH dependent, whereas mmsB and NDSD from Pseudomonas can utilize either NADPH or NADH.

The malonate semialdehyde enol form is converted to 3-HP utilizing an N-ethylmaleimide reductase (nemA, SEQ ID NO. 17 nucleic acid, SEQ ID NO. 18 protein), and/or a malonic semialdehyde reductase (rutE, SEQ ID NO. 19 nucleic acid, SEQ ID NO. 20 protein) from E. coli. These enzymes does not directly utilize NADPH or NADH. Instead, these enzymes utilize a flavin mononucleotide that is cycled between oxidized and reduced states by NADPH or NADH. The enol pathway also has advantages over the keto pathway in that equilibrium between the malonate semialdehyde enol form and 3-HP significantly favors 3-HP, making the reaction much less reversible, and essentially irreversible.

The malonate semialdehyde hydrated form may also be converted to 3-HP by either the 3-HP dehydrogenase or malonate semialdehyde reductase enzymes, although the hydrated form is more likely to be converted to the enol form as the equilibrium continuously readjusts.

In one embodiment, the microorganism herein comprise a genetic modification that include (i.e., microorganism) includes a polynucleotide encoding: (1) a monofunctional malonyl-CoA reductase gene capable of catalyzing the conversion of malonyl-CoA to malonate semialdehyde; and (2) one or more genes encoding one or more of the following enzymes: ydfG, mmsB, NDSD, rutE, and nemA or a functional homolog or a homolog with at least 80% identity.

In accordance with another aspect of the invention, there is provided an organism that is genetically modified to make 3-HP, wherein the genetic modification includes a polynucleotide encoding: (1) a monofunctional malonyl-CoA reductase gene capable of catalyzing the conversion of malonyl-CoA to malonate semialdehyde; (2) one or more genes encoding one or more enzymes capable of converting malonate semialdehyde keto form to 3-HP; and (3) one or more genes encoding one or more enzymes capable of converting either the malonate semialdehyde enol form or the malonate semialdehyde hydrated form to 3-HP.

In certain aspects the invention provides monofunctional malonyl-CoA reductase enzyme fused to a dehydrogenase enzyme that is either: (1) primarily not NADPH-dependent; (2) primarily NADH-dependent; (3) primarily flavin-dependent; (4) less susceptible to 3-HP inhibition at high concentration; and/or (5) catalyzes a reaction pathway to 3-HP that is substantially irreversible.

In certain aspects the invention also provides monofunctional malonyl-CoA reductase enzyme fused to a dehydrogenase enzyme that is NADPH-dependent.

Suitable 3-HP dehydrogenase enzymes that are largely NADH-dependent that can be used with the claimed invention include, but are not limited to, mmsB or NDSD. Suitable malonate reductase enzymes that are flavin-dependent include, but are not limited to, rutE and nemA. Suitable 3-HP dehydrogenase enzymes that are less susceptible 3-HP inhibition at high concentration that can be used with the claimed invention include, but are not limited to, ydfG and NDSD. Suitable 3-HP dehydrogenase or malonate semialdehyde dehydrogenase enzymes that catalyze a reaction pathway to 3-HP that is substantially irreversible are rutE and nemA.

In certain aspects the invention provides monofunctional malonyl-CoA reductase enzyme fused to one or more dehydrogenase enzymes. Malonate semialdehyde, which is the intermediate product in the conversion of malonyl-CoA to 3-HP can be very reactive. Therefore, it is advantageous to have a reaction pathway wherein the residence time of malonate semialdehyde within the cell is minimized, and its conversion to 3-HP occurs quickly. By fusing the malonyl-CoA reductase with the malonate semialdehyde dehydrogenase to create a multi-domain protein (e.g., two domain protein) and having the MCR and dehydrogenase domains adjacent in the sequence, when the themalonate semialdehyde is quickly is quickly converted to 3-HP.

In certain aspects the invention provides first monofunctional malonyl-CoA reductase enzyme fused to a first dehydrogenase enzyme of one type and second monofunctional malonyl-CoA reductase enzyme fused to a dehydrogenase enzyme of a different type than the first dehydrogenase enzyme. Suitable different dehydrogenase enzymes include, but are not limited to, enzymes that function on the different forms of malonate semialdehyde.

In certain aspects the invention provides for microorganisms comprising a genetic modification that include but are not limited to the malonyl-CoA reductase from S. tokadaii is fused to ydfG, mmsB, NDSD, rutE, or nemA (or some combination thereof). The fused enzyme may include any of the following configurations: mcr-ydfG, mcr-mmsB, mcr-NDSD, mcr-rutE, mcr-nemA, mcr-ydfG-mmsB, mcr-ydfG-NDSD, mcr-ydfG-rutE, mcr-ydfG-nemA, mcr-mmsB-ydfG, mcr-mmsB-NDSD, mcr-mmsB-rutE, mcr-mmsB-nemA, mcr-NDSD-ydfG, mcr-NDSD-mmsB, mcr-NDSD-rutE, mcr-NDSD-nemA, mcr-rutE-ydfG, mcr-rutE-mmsB, mcr-rutE-NDSD, mcr-rutE-nemA, mcr-nemA-ydfG, mcr-nemA-mmsB, mcr-nemA-NDSD, or mcr-nemA-rutE or functional homolog or homolog with 80% sequence identity thereof.

In certain aspects the invention provides for microorganisms comprising a genetic modification that include but are not limited to the malonyl-CoA reductase from C. aggregans is fused to ydfG, mmsB, NDSD, rutE, or nemA (or some combination thereof). The fused enzyme may include any of the following configurations: mcr-ydfG, mcr-mmsB, mcr-NDSD, mcr-rutE, mcr-nemA, mcr-ydfG-mmsB, mcr-ydfG-NDSD, mcr-ydfG-rutE, mcr-ydfG-nemA, mcr-mmsB-ydfG, mcr-mmsB-NDSD, mcr-mmsB-rutE, mcr-mmsB-nemA, mcr-NDSD-ydfG, mcr-NDSD-mmsB, mcr-NDSD-rutE, mcr-NDSD-nemA, mcr-rutE-ydfG, mcr-rutE-mmsB, mcr-rutE-NDSD, mcr-rutE-nemA, mcr-nemA-ydfG, mcr-nemA-mmsB, mcr-nemA-NDSD, or mcr-nemA-rutE or functional homolog or homolog with 80% sequence identity thereof.

In certain aspects the invention provides for microorganisms comprising a genetic modification that include but are not limited to the malonyl-CoA reductase from O. trichoides is fused to ydfG, mmsB, NDSD, rutE, or nemA (or some combination thereof). The fused enzyme may include but are not limited to any of the following configurations: mcr-ydfG, mcr-mmsB, mcr-NDSD, mcr-rutE, mcr-nemA, mcr-ydfG-mmsB, mcr-ydfG-NDSD, mcr-ydfG-rutE, mcr-ydfG-nemA, mcr-mmsB-ydfG, mcr-mmsB-NDSD, mcr-mmsB-rutE, mcr-mmsB-nemA, mcr-NDSD-ydfG, mcr-NDSD-mmsB, mcr-NDSD-rutE, mcr-NDSD-nemA, mcr-rutE-ydfG, mcr-rutE-mmsB, mcr-rutE-NDSD, mcr-rutE-nemA, mcr-nemA-ydfG, mcr-nemA-mmsB, mcr-nemA-NDSD, or mcr-nemA-rutE or functional homolog or homolog with 80% sequence identity thereof.

Enhanced Mutated Monofunctional MCR for Bioproduction

In certain aspects the invention provides for microorganisms comprising a genetic modification that include mutated form of stMCR that has enhanced activity at about 20° C. to about 44° C., about 30° C. to about 37° C., or about 32° C. to about 38° C. Such mutate forms may be designed based on the crystal structure now available for stMCR [Demmer et al., J. Biol. Chem. 288:6363-6370, 2013].

It is also contemplated the carboxylase domains of the malonyl-CoA reductase derived from Chloroflexus aggregans, Oscillochloris trichoides can be enhanced by mutations in the carboxylase binding domain to provide increased 3-HP production over the natural occurring enzyme.

The carboxylase activity of the malonyl-CoA reductase derived from Chloroflexus aurantiacus can be enhanced activity. In certain aspects the invention provides for mutated form of it carboxylase domain to provide increased 3-HP production over the natural occurring enzyme.

In certain aspects the invention provides for microorganisms comprising a genetic modification that include carboxylase domains of the malonyl-CoA reductase derived from C. aggregans is fused to ydfG, mmsB, NDSD, rutE, or nemA (or some combination thereof). It is contemplated that the any of the enhanced MCR by mutation, as provide above, may be fused in any of the following configurations including but not limited to mcr-ydfG, mcr-mmsB, mcr-NDSD, mcr-rutE, mcr-nemA, mcr-ydfG-mmsB, mcr-ydfG-NDSD, mcr-ydfG-rutE, mcr-ydfG-nemA, mcr-mmsB-ydfG, mcr-mmsB-NDSD, mcr-mmsB-rutE, mcr-mmsB-nemA, mcr-NDSD-ydfG, mcr-NDSD-mmsB, mcr-NDSD-rutE, mcr-NDSD-nemA, mcr-rutE-ydfG, mcr-rutE-mmsB, mcr-rutE-NDSD, mcr-rutE-nemA, mcr-nemA-ydfG, mcr-nemA-mmsB, mcr-nemA-NDSD, or mcr-nemA-rutE or functional homolog or homolog with 80% sequence identity thereof.

IV. Salt-Tolerant Enzymes

The growth of engineered microorganism for enhanced production of a chemical product, such as E. coli is severely inhibited by high salt concentrations accumulated when the chemical product is produced at high rate within the organism.

Dose-dependent studies with increasing amounts of NaCl and Na-3-HP show that salt has inhibitory effects on ACCase activity which is essential to fatty acid biosynthesis of membranes required for growth and propagation and for the production of 3-HP (see EXAMPLE 1). Thus, the use of salt-tolerant enzymes in 3-HP production should increase 3-HP production in a host cell.

A. Enzymes from Halophilic Organisms

Halophiles are characterized as organisms with a great affinity for salt. In some instances a halophilic organism is one that requires at least 0.05M, 0.1M, 0.2M, 0.3M or 0.4M concentrations of salt (NaCl) for growth. Halophiles live in hypersaline environments that are generally defined occurring to their salt concentration of their habitats. Halophilic organisms that are defined as “Slight salt affinity” have optimal growth at 2-5% NaCl, moderate halophiles have optimal growth at 5-20% NaCl and extreme halophiles have optimal growth at 20-30% NaCl.

Depending on the conditions of that the genetically engineered microorganism is under one might use homologous enzymes of the invention specifically, for example, from a moderate halophiles or an extreme halophiles depending on the engineered cell's environment.

In certain aspects the invention provides for microorganisms comprising a genetic modification that includes enzymes of the invention provided herein from slight halophiles organisms. In certain aspects the invention provides for microorganisms comprising a genetic modification that includes enzymes of the invention provided herein from moderate halophiles organisms. In certain aspects the invention provides for microorganisms comprising a genetic modification that includes homologous enzymes of the invention provided herein from extreme halophiles organisms.

Homology with genes provided by the invention may be determined by analysis with BLASTN version 2.0 provided through the NCBI website. Homology with proteins provided by the invention may be determined by analysis with BLASTP version 2.2.2 provided through the NCBI website. This program with aligns the disclosed fragments being compared and determines the extent of identity or similarity between them.

To date there are many sequenced halophilic organisms which can be used with the claimed invention. Examples of some sequenced halophilic organisms include but are not limited to Chromohalobacter salexigens, Flexistipes sinusarabici strain (MASIOT), Halobacterium sp. NRC-1, Haloarcula marismortui, Natronomonas pharaonis, Haloquadratum walsbyi, Haloferax volcanii, Halorubrum lacusprofundi, Halobacterium sp. R-1, Halomicrobium mukohataei, Halorhabdus utahensis, Halogeometricum borinquense, Haloterrigena turkmenica, Natronobacterium gregoryi SP2, Halalkalicoccus jeotgali, Natrialba magadii, Haloarcula hispanica, Halopiger xanaduensis, Halophilic archaeon DL31, Haloferax mediterranei, Halovivax ruber, Natronococcus gregoryi, and Natronococcus occultus.

Examples of suitable moderate halophilic organisms in which homologous enzymes of the invention can be derived from include but are not limited to eukaryotes such as crustaceans (e.g. Artemia salina), insects (e.g. Ephydra hians), certain plants from the genera Salicornia spp, algae (e.g. Dunaliella viridis), fungi, and protozoa (e.g. Fabrea salina), phototrophic organisms, such as planktonic and microbial mat-formers cyanobacteria as well as other anaerobic red and green sulphur bacteria from the genera Ectothiorhodospira spp.) and non-sulphur bacteria from the genera Chromatium spp.; gram-negative anaerobic bacteria, for example from the genera Haloanaerobacter spp. some of which are methanogenic, for example from the genera Methanohalophilus spp. and either aerobic or facultative such as species from the genera Halomonas, Chromohalobacter, Salinovibrio, Pseudomonas, for example (e.g. Halomonase elongate); gram-positive bacteria from genera such as Halobacillus, Bacillus, Marinococcus, etc. as well as some actinomycetes, for example, Actinopolyspora halophila.

### Genomic and Proteomic Hallmarks of Halophilic Organisms

Comparative genomic and proteomic studies of halophiles and non-halophiles reveal some common trends in the genomes and proteomes of halophiles. At the protein level, halophilic organisms are characterized by low hydrophobicity, over-representation of acidic residues, especially Asp, under-representation of Cys, lower propensities for helix formation and higher propensities for coil structure.

At the DNA level, halophilic organisms are characterized by the dinucleotide abundance profiles of halophilic genomes bear some common characteristics, which are quite distinct from those of non-halophiles, and hence may be regarded as specific genomic signatures for salt-adaptation. The synonymous codon usage in halophiles also exhibits similar patterns regardless of their long-term evolutionary history.

In certain aspects the invention provides for microorganisms comprising a genetic modification that the proteins provided by the invention that are modified for salt tolerance such that they has low hydrophobicity, over-representation of acidic residues, especially Asp, under-representation of Cys, lower propensities for helix formation and higher propensities for coil structure.

Suitable salt-tolerant enzymes can include enzymes from salt-tolerant organisms. Salt-tolerant organisms (such as, for example, halophiles) include any living organism that are adapted to living in conditions of high salinity. Suitable salt-tolerant enzymes can include enzymes from salt-tolerant organism that are homologs of the following enzymes: Sucrose-6-phosphate hydrolase (cscA from E. coli), glucose-6-phosphate isomerase (pgi from E. coli), fructokinase (cscK from E. coli), fructose-1,6-bisphosphatase (yggF from E. coli), fructose 1,6-bisphosphatase (ybhA from E. coli), fructose 1,6-bisphosphatase II (glpX from E. coli), fructose-1,6-bisphosphatase monomer (fbp from E. coli), 6-phosphofructokinase-1 monomer (pfkA from E. coli), 6-phosphofructokinase-2 monomer (pfkB from E. coli), fructose bisphosphate aldolase monomer (fbaB from E. coli), fructose bisphosphate aldolase monomer (fbaA from E. coli), triose phosphate isomerase monomer (tpiA), glyceraldehyde 3-phosphate dehydrogenase-A monomer (gapA from E. coli), phosphoglycerate kinase (pgk), 2,3-bisphosphoglycerate-independent phosphoglycerate mutase (gpmM from E. coli), 2,3-bisphosphoglycerate-dependent or tdcE (from E. coli), phosphoglycerate mutase (gpmA), enolase (eno from E. coli), phosphoenolpyruvate carboxylase (ppc from E. coli), malate dehydrogenase (mdh), fumarase A (fum from E. coli), fumarase B (fumB), fumarase C (fumC from E. coli), phosphoenolpyruvate synthetase (ppsA from E. coli), pyruvate kinase I monomer (pykF from E. coli), pyruvate kinase II monomer (pykA from E. coli), fumarate reductase (frdABCD from E. coli), lipoamide dehydrogenase (lpd from E. coli), pyruvate dehydrogenase (aceE from E. coli), pyruvate dehydrogenase (aceF from E. coli), pyruvate formate-lyase (pflB from E. coli), acetyl-CoA carboxylase (accABCD from E. coli), malonyl CoA reductase (mcr), 3HP dehydrogenase (mmsB, NDSD, or ydfG), malonate semialdehyde reductase (nemA, rutE from E. coli), or a combination thereof.

Suitable salt-tolerant enzyme homologs that can be used with the claimed invention can have at least 99%, 98%, 97%, 96%, 95%, 94%, 93%, 92%, 91%, 90%, 89%, 88%, 87%, 86%, 85%, 84%, 83%, 82%, 81% or 80%, overall amino acid or nucleotide identity to the above enzymes. Suitable salt-tolerant enzyme homologs that can be used with the claimed invention can have at least 99%, 98%, 97%, 96%, 95%, 94%, 93%, 92%, 91%, 90% 89%, 88%, 87%, 86%, 85%, 84%, 83%, 82%, 81% or 80%, amino acid or nucleotide to the essential protein function domains of the enzymes above. Suitable salt-tolerant enzyme homologs that can be used with the claimed invention can have at least 99%, 98%, 97%, 96%, 95%, 94%, 93%, 92%, 91%, 90%, 89%, 88%, 87%, 86%, 85%, 84%, 83%, 82%, 81% or 80% overall amino acid or nucleotide to the essential binding amino acids within an essential protein function domain of the enzymes above.

In accordance with a preferred embodiment of the invention, suitable salt-tolerant enzyme homologs are enzymes from one of the following organisms: Halomonas elongata, Salinibacter rubur, or Halobacterium species (Archaea).

In accordance with a preferred embodiment of the present invention, there is provided a non-salt-tolerant organism that is genetically modified to make 3-HP, wherein the genetic modification includes a polynucleotide encoding an acetyl-CoA carboxylase from a salt-tolerant organism. In accordance with a preferred embodiment, the acetyl-CoA carboxylase subunits accA, accB, accC and accD is from Halomonas elongata.

V. Chemical Transporter

In accordance with another aspect of the present invention, any of the microorganisms herein may be genetically modified to introduce a nucleic acid sequence coding for a polypeptide that: (1) facilitates the exportation of the chemical of interest or the export of an inhibitory chemical from within the cell to the extracellular media; and/or (2) facilitates the importation from the extracellular media to within the cell of a reactant, precursor, and/or metabolite used in the organism's production pathway for producing the chemical of interest.

3-HP Exporter

In accordance with a preferred embodiment, this invention relates to the bioproduction of 3-HP using a genetically modified E. coli organism. Thus, the present invention contemplates of a host cell genetically modified to express or increase expression of an exporter that can function to transfer 3 HP from the cellular environment extracellularly.

Bacterial cells, such as E. coli, have at least five different types of exporters: the major facilitator superfamily (MFS); the ATP-binding cassette superfamily (ABC); the small multidrug resistance family (SMR); the resistance-nodulation-cell division superfamily (RND); and the multi antimicrobial extrusion protein family (MATE). In addition, amino acid exporters, which are common to almost all host cells, are likely to export 3-HP. Additionally, solvent tolerance transporters, for example bromoacetate, butanol, isobutanol and the alike may be used to export 3-HP.

In certain aspects the invention provides a host cell with a recombinant exporter wherein the exporter is an MFS exporter, ABC exporter, SMR exporter, RND exporter, MATE exporter, amino acid exporter, solvent tolerance transporter or a combination thereof.

Suitable exporters that can be used with the s herein invention include but are not limited to acrD, bcr, cusA, dedA, eamA, eamB, eamH, emaA, emaB, emrB, emrD, emrKY, emrY, garP, gudP, hsrA, leuE, mdlB, mdtD, mdtG, mdtL, mdtM, mhpT, rhtA, rhtB, rhtC, thtB, yahN, yajR, ybbP, ybiF, ybjJ, ycaP, ydcO, yddG, ydeD, ydgE, yddG, ydhC, ydhP, ydiN, ydiM, ydjE, ydjI, ydjK, yeaS, yedA, yeeO, yegH, yggA, yfcJ, yfiK, yhjE, yidE, yigK, yigJ, yijE, yjil, yjiJ, yjiO, ykgH, ypjD, ytfF, ytfL or functional homolog or homolog with 80% sequence identity thereof. Other potential transporter proteins may be identified using topology analysis as illustrated in [Daley et al., Science 308: 1321-1323, 2005].

In certain aspects the invention provides the exporter to be improved for binding to 3-HP. In certain aspects the invention provides the exporters named to be further enhance by genetic modification of the predicted cytoplasmic domain to increase 3-HP binding. In certain aspects the invention provides the exporter to be improved for binding to 3-HP. In certain aspects the invention provides the exporters named to be further enhance by genetic modification of the predicted transmembrane binding domain to increase 3-HP binding or incorporation into the host cell membrane.

Suitable exporter homologs that can be used with the claimed invention can have at least 99%, 98%, 97%, 96%, 95%, 94%, 93%, 92%, 91%, 90%, 89%, 88%, 87%, 86%, 85%, 84%, 83%, 82%, 81% or 80% overall amino acid or nucleotide identity to the above exporters. Suitable exporter homologs that can be used with the claimed invention can have 99%, 98%, 97%, 96%, 95%, 94%, 93%, 92%, 91%, 90%, 89%, 88%, 87%, 86%, 85%, 84%, 83%, 82%, 81% or 80% amino acid or nucleotide to the essential protein function domains of the exporters above. Suitable exporter homologs that can be used with the claimed invention can have at least 99%, 98%, 97%, 96%, 95%, 94%, 93%, 92%, 91%, 90%, 89%, 88%, 87%, 86%, 85%, 84%, 83%, 82%, 81% or 80% overall amino acid or nucleotide to the essential binding amino acids within an essential exporter domain of the enzymes above.

In certain aspects the invention provides for at least of the exporters provided herein to be expressed in a host cell to increase the chemical production of 3-HP in a host cell. In certain aspects the invention provides for at least of the exporters provided herein to be expressed in a host cell and with a genetic modification of tig to increase the chemical production of 3-HP in a host cell.

In certain aspects the invention provides for one exporter to be further modified by on one more genetic modulates so that the expression level and timing of expression of the exporter can be controlled in the host cell. In certain aspects the invention provides for one exporter to be further modified by an inducible promoter, RBS, high, mutlicopy plasmid or combination thereof, as provide herein, in order to control its expression in the host cell.

In certain aspects the invention provides exporters provide herein to be expressed in a host cell in equal ratio. In certain aspects the invention provides exporters provide herein to be expressed in a host cell in equal 1:2 ratio. In certain aspects the invention provides exporters provide herein to be expressed in a host cell in equal 1:3 ratio. In certain aspects the invention provides exporters provide herein to be expressed in a host cell in equal 1:4 ratio. In certain aspects the invention provides exporters provide herein to be expressed in a host cell in equal 2:3 ratio.

In certain aspects the invention provides for the exporter to maintain the host cell at pH 7.0-7.4 during the continuous production phase. In certain aspects the invention provides for the exporter and the means for importing a base inside the cell in order to maintain the host cell at pH 7.0-7.4 during the continuous production phase. In certain aspects the invention provides for the exporter maintain the host cell at pH 3.0 to pH 4.0, pH 4.0 to pH 5.0, pH 5.0 to pH 6.0, pH 6.0 to pH 7.0, pH 7.0 to pH 8.0, pH 8.0 to pH 9.0, or pH 9.0 to pH 10.0 pH 7.0-7.3 during the continuous production phase. In certain aspects the invention provides for the exporter and the means for importing a base inside the cell in order to maintain the host cell at pH 3.0 to pH 4.0, pH 4.0 to pH 5.0, pH 5.0 to pH 6.0, pH 6.0 to pH 7.0, pH 7.0 to pH 8.0, pH 8.0 to pH 9.0, or pH 9.0 to pH 10.0 pH 7.0-7.3 during the continuous production phase.

In accordance with this aspect of the present invention, addition modifications to the host cell may be made to further enhance the transporter's function. In particular, deletion of the tig gene from the genome of the host cell may enhance expression and total activity of integral membrane proteins such as exporters and importers.

Bicarbonate Importer

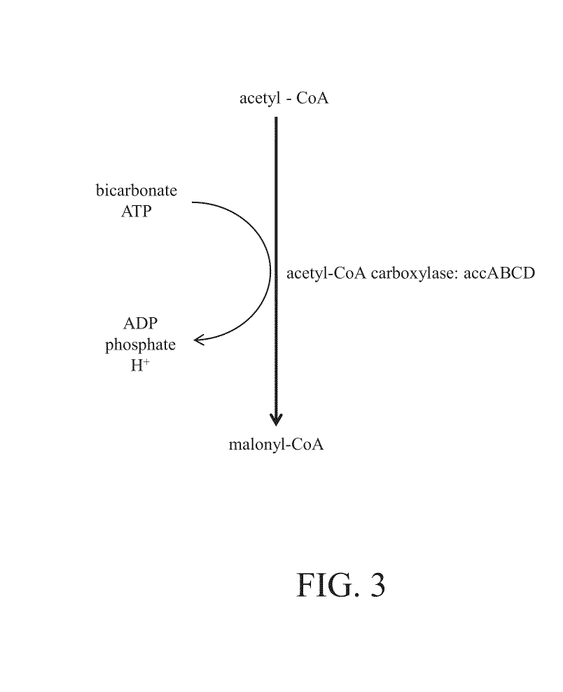

One of the key steps in the conversion of biomass to 3-HP is the conversion of acetyl-CoA to malonyl-CoA, which is illustrated in FIG. 3.

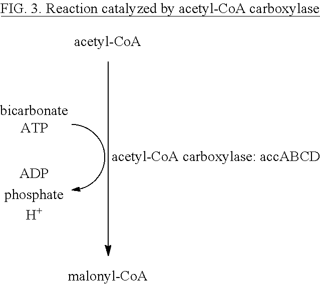

As shown in FIG. 3, this reaction is catalyzed by the acetyl-CoA carboxylase, and bicarbonate is a reactant needed to drive the reaction. One of the primary sources of bicarbonate to drive this reaction is carbon dioxide within the cell. Carbon dioxide is readily diffusible through a cell's membrane, and a natural equilibrium will be reached between the intracellular and extracellular carbon dioxide. As a cell produces carbon dioxide it migrates through the cell, and since it is not very soluble in the media, it will bubble out of the system and more intracellular carbon dioxide will migrate out of the cell to maintain the equilibrium. This process impedes the production of 3-HP since bicarbonate (which is in equilibrium with the dissolved carbon dioxide in the form of carbonic acid) is needed to drive the acetyl-CoA malonyl-CoA reaction, and the intracellular carbon dioxide is the primary source for intracellular bicarbonate.

In accordance with one aspect of the present invention, an organism is provided that includes a heterologous gene encoded therein that acts as a carbon dioxide importer (i.e., it enhances the importation of carbon dioxide into the cell or inhibits the exportation of carbon dioxide from the cell), which results in increased intracellular carbon dioxide. Use of CO2 an importer mitigates against the natural outflow of carbon dioxide.

In accordance with this aspect of the invention, there is provided an organism that is genetically modified, wherein the genetic modification includes a polynucleotide encoding a gene capable of importing extracellular carbon dioxide from the media to within the cell membrane or inhibiting the exportation of intracellular carbon dioxide from within the cell membrane to the media. In accordance with a preferred embodiment of the present invention, a microorganism is genetically modified to encode one or more of the following heterologous genes: bicA from Synechococcus species, ychM gene product of E. coli, yidE gene product of E. coli, any of the bicarbonate transporters as described in [Felce and Saier, J. Mol. Microbiol. Biotechnol. 8: 169-176, 2004 or any amino acid sequences homologous thereof (e.g., at least 80%, 85%, 90%, 95%, or 99% homologous to the amino acids sequences of the CO2 importer/exporters described herein].

Bioproduction Methods

In some applications of the invention the host cell is genetically modified for increased malonyl-CoA flux by at least one heterologous ACCase complex, such as Table 4 to further increase chemical bio-production in host cell. In some applications of the invention the host cell is genetically modified with heterologous salt tolerant enzymes, such as Table 5 to increase chemical bio-production in a host cell. In some applications of the invention the host cell is genetically modified with heterologous 3-HP exporters to further increase chemical bio-production in a host cell.

In some applications of the invention the host cell is genetically modified by at least one heterologous gene and/or salt tolerant heterologous gene of FIG. 1 or Table 5 and at least one 3-HP exporter provided herein to further increase chemical bioproduction in a host cell.

In some applications of the invention the host cell is genetically modified with a heterologous gene for increased malonyl-CoA flux, 3-HP export, at least one heterologous and/or salt tolerant heterologous gene, as provided herein, to increase chemical bio-production in a host cell. In some applications of the invention the host cell is genetically modified for increased malonyl-CoA flux, 3-HP export, at least one heterologous gene and/or salt tolerant heterologous gene and the host cell is genetically modified by at least one gene, as provided herein to increase chemical bioproduction in a host cell.

When utilizing certain organisms to create certain products, it may be advantageous to control each phase discretely. For example, depending on the pathway involved, reactions, reactants, intermediates and byproducts created during cell growth can inhibit enzyme induction and/or the organism's ability to produce the desired chemical product. Similarly, reactions, reactants, intermediates and byproducts created as part of the production pathway can impact cell growth, and even the increased concentration of the chemical product as it is produced can impede cell replication.

### Table. 5

VI. Multi-Phase Fermentation

In accordance with another aspect of the present invention, there is provided a method of producing a chemical product from a carbon source through a bioproduction process that comprises a controlled multi-phase production process. The multi-phase production process includes an initiation and/or completion of one or more phases of the production process is controlled by genetic modifications to the organism producing the chemical product and/or is controlled by changes made to the cell environment.

In accordance with this aspect of the invention, the bioproduction process may include two or more of the following phases: (1) growth phase; (2) induction phase; and (3) production phase. During the growth phase, the organism replicates itself and the biocatalyst needed to produce the chemical product is built up. During the induction phase, expression of key enzymes critical to the production of the chemical is induced and the enzymes accumulate within the biocatalyst to carry out the reactions required to produce the product. During the production phase organism produces the desired chemical product.

The initiation and/or completion of the growth, induction and/or production phases are controlled. In accordance with the present invention, the growth phase is dependent on the presence of a critical external reactant that will initiate growth. The initiation and completion of the growth phase is controlled by the addition and amount of the initiating reactant added to the reaction vessel.

In accordance with certain preferred embodiments of the present invention, the chemical product is 3-HP and the production organism is E. coli or yeast. The critical external reactant may be phosphate, which is needed for replication of E. coli cells. In accordance with a preferred embodiment, the growth phase is initiated by the addition of phosphate to a reaction vessel (together with a carbon source such as sugar and the E. coli cells), and the duration of the growth phase is controlled by the amount of phosphate added to the system.

The induction phase is controlled by genetic modifications to the producing organism. The key enzymes triggered during this phase are engineered into the organism using promoters that are sensitive to (e.g., activated by) the depletion of the initiating reactant. As a result, once the initiating reactant is depleted, the growth phase ends, the key enzymes are activated and the induction phase begins.

In accordance with a preferred embodiment, the induction phase is controlled by key genes that encode for enzymes in the biosynthetic pathway for the product into the production organism using promoters that are activated by phosphate depletion. In one embodiment where the chemical product is 3-HP and the production organism is E. coli, the key genetic modifications may include one or more of the following: mcr, mmsB, ydfG, rutE, nemA and NDSD; genes that encode individual or fused subunits of ACCase, such as accA, accB, accC, accD, accDA fusion, and accCB fusion, and the promoters may include one or more of the promoters that direct expression of the following E. coli genes: amn, tktB, xasA, yibD, ytfK, pstS, phoH, phnC, or other phosphate-regulated genes as described in [Baek and Lee, FEMS Microbiol Lett 264: 104-109, 2006]. In accordance with this embodiment, once the phosphate is depleted, expression of the key enzymes is activated by their promoters and the induction phase begins.

The production phase may also be controlled by genetic modifications. For example, the organism can be engineered to included mutated forms of enzymes critical to the initiation of production of the chemical product. These initiation enzymes may facilitate initiation of production either by: (1) becoming active and serving a key function in the production pathway; and/or (2) becoming inactive and thereby turning off a branch pathway or other competitive pathway that prevents or limits the production pathway leading to the chemical product. In accordance with a preferred embodiment, initiation enzymes are mutated to form temperature sensitive variants of the enzymes that are either activated by or deactivated at certain temperatures. As a result, the production phase is initiated by changing the changing the temperature within the reaction vessel.

In one embodiment, the production phase is controlled by genetically modifying the microorganism with a heterologous nucleotide sequence encoding i one or more of the following temperature sensitive enzymes: fabI^{ts} (SEQ ID NO. 27), fabB^{ts} (SEQ ID NO. 28) and fabD^{ts} (SEQ ID NO. 29). These enzymes are deactivated or shut-off at the desired temperature for production of the chemical product. These enzymes play a key role shuttling carbon atoms into the fatty acid synthesis pathway. Although fatty acid synthesis pathway is critical during the growth phase, it inhibits production of the chemical product. Once the reaction vessel temperature is changed, the temperature sensitive enzymes are deactivated and the fatty acid synthesis pathway shuts down thereby allowing the production pathway of the chemical product to ramp up.

In accordance with the present invention, the growth phase can last be between 10 to 40 hours, or about 15 to about 35 hours, or about 20 to about 30 hours. The induction phase may be for about 1 to about 6 hours, about 1 to about 5 hours, or about 2 to about 4 hours. The production phase may be greater than 5, 10, 15, 20, 25, 30, 35, 40, 45, 50, 55, 60, 65, 70, 75, 80, 85, 90, 95 or 100 hours depending on the amount of chemical product that is desired.

In accordance with the present invention, the growth phase and induction phase are conducted at a temperature of about 25° C. to about 35° C., about 28° C. to about 32° C., or about 30° C. The production phase is conducted at a temperature of about 35° C. to about 45° C., about 35° C. to about 40° C., or about 36° C. to about 38° C. Preferably, the production phase temperature is higher than the induction phase temperature, and the increase in temperature that initiates the production phase occurs over a period of about 1 to about 5 hours, about 1 to about 3 hours, about 2 hours, or about 1 hour.

In accordance with the present invention, there is provided a method of producing a chemical product from a renewable carbon source through a bioproduction process comprising:
(1) constructing a genetically modified organism capable of converting said renewable carbon source to said chemical product, wherein said genetically modified organism requires inorganic phosphate for growth and comprises: (a) at least one heterologous gene whose expression is regulated by a promoter sensitive to inorganic phosphate levels within a culture system, wherein said gene provides a critical function in converting said carbon source to said chemical product and is not required for the genetically modified organism to replicate; and (b) a gene encoding a temperature-sensitive enzyme;
(2) forming a culture system comprising said carbon source in an aqueous medium and said genetically modified microorganism;
(3) maintaining the culture system under conditions that allow the genetically modified microorganism to replicate comprising maintaining a sufficient level of inorganic phosphate within said culture system;
(4) allowing the inorganic phosphate to deplete thereby triggering the expression of the gene regulated by a promoter sensitive to inorganic phosphate levels; and
(5) changing the temperature of the culture system thereby activating or deactivating said temperature-sensitive enzyme and initiating the production of said chemical product.

In accordance with the present invention, there is provided a method of producing 3-hydropropionic acid (3-HP) from a renewable carbon source, comprising:
(1) constructing a genetically modified organism capable of converting said renewable carbon source to 3-HP, wherein said genetically modified organism requires inorganic phosphate for growth and comprises: (a) at least one heterologous gene whose expression is regulated by a promoter sensitive to inorganic phosphate levels within a culture system, wherein said gene is selected from the group consisting of mcr, mmsB, ydfG, rutE, nemA, NDSD, accA, accB, accC, accD, accDA fusion, and accCB fusion; and (b) a gene encoding a temperature-sensitive enzyme selected from the group consisting of fabI, fabB and fabD;
(2) forming a culture system comprising said carbon source in an aqueous medium, phosphate and said genetically modified microorganism, and thereby initiating a growth phase during which the genetically modified microorganism replicates;
(3) maintaining a sufficient level of inorganic phosphate within said culture system until the desired level of cell growth is achieved;
(4) allowing the inorganic phosphate to deplete thereby initiating an induction phase which begins the expression of said gene regulated by a promoter sensitive to inorganic phosphate levels; and
(5) changing the temperature of the culture system thereby activating or deactivating said temperature-sensitive enzyme and initiating a growth phase during which said genetically modified microorganism produces 3-HP.

### Fermentation Conditions

Depending on the host cell fermentation may be performed under aerobic, microaerobic, or anaerobic conditions, with or without agitation. The operation of culture systems to achieve aerobic, microaerobic and anaerobic conditions are well known to those of ordinary skill in the art.

Suitable pH ranges for fermentation depend on the multiple factors such as the host cell. In some applications of the invention fermentation can occur between various pH ranges for example, pH 3.0 to pH 4.0, pH 4.0 to pH 5.0, pH 5.0 to pH 6.0, pH 6.0 to pH 7.0, pH 7.0 to pH 8.0, pH 8.0 to pH 9.0, or pH 9.0 to pH 10.0. However, the actual pH conditions for a particular application are not meant to be limited by these ranges and can be between the expressed pH ranges if it provides more optimal production of the fermentation process, such as increased 3-HP production.

VII. Genes and Proteins for the Bioproduction of Chemicals

An overview of the engineered pathways provided by the invention in a host cell is shown in FIG. 1. Various combinations of the pathways shown can be carried out by various combinations of genetic modifications to key enzymes either in the intrinsic pathways or supplied through the transformation of a heterologous gene.

In some applications of the genetically modified microorganism of the invention may comprise a single genetic modification, or one or more genetic modifications. Various types of genetic modifications that can be used with the invention are disclosed herein.

In some embodiments the genetic modified organism of the invention can comprise a genetic modification to the following gene/proteins or a homolog with at least 80% identity to or a functional homolog of: bifunctional malonyl-CoA reductase (MCR from Chloroflexus aurantiacus), monofunctional malonyl-CoA reductase (caMCR from Chloroflexus aurantiacus), malonyl-CoA reductase (stMCR from Sulfolobus tokodaii), Enzyme: malonyl-CoA reductase (cgMCR from Chloroflexus aggregans), Enzyme: malonyl-CoA reductase (otMCR from Oscillochloris trichoides), Polypeptide: host restriction; endonuclease R (hsdR from E. coli), lactose metabolism (lac from E. coli), L-rhamnulose kinase (rhaB from E. coli), rhamnulose-1-phosphate aldolase (rhaD from E. coli), Enzyme: β-galactosidase (lacZ from E. coli), L-ribulose 5-phosphate 4-epimerase (araD from E. coli), L-ribulokinase (araB from E. coli), Enzyme: D-lactate dehydrogenase—fermentative (ldhA from E. coli), enzyme: pyruvate formate-lyase (pflB from E. coli), Enzyme: phosphate acetyltransferase/phosphate propionyltransferase (pta from E. coli), Enzyme: pyruvate oxidase (poxB from E. coli), Enzyme: methylglyoxal synthase (mgsA from E. coli), enzyme: Acetate kinase (ackA from E. coli), enzymes: phosphotransacetylase-acetate kinase (pta-ack from E. coli), Enzyme: enoyl-[acyl-carrier-protein] reductase (fabI from E. coli), Protein: zeocin binding protein (zeoR from Streptoalloteichus Hindustanus), Enzyme: carboxytransferase moiety of acetyl-CoA carboxylase (accAD from E. coli), Enzyme: triose phosphate isomerase (tpiA from E. coli), Enzyme: biotoin carboxylase moiety of acetyl-CoA carboxylase (accBC from E. coli), Enzyme: transhydrogenase (pntAB from E. coli), Polypeptide: Lad DNA-binding transcriptional repressor (lacI from E. coli), Enzyme: β-ketoacyl-ACP synthases I (fabB from E. coli), Enzyme: β-ketoacyl-ACP synthases II (fabF from E. coli), Enzyme: malonyl-CoA-ACP transacylase (fabD from E. coli), Enzyme: pantothenate kinase (coaA from E. coli), Enzyme: pyruvate dehydrogenase complex (aceEF from E. coli), Enzyme: 3-hydroxyisobutyrate/3-HP dehydrogenase (mmsB from Pseudomonas aeruginosa), Enzyme: lipoamide dehydrogenase (lpd from E. coli), Enzyme: γ-glutamyl-γ-aminobutyraldehyde dehydrogenase (puuC from E. coli), Enzyme: malate synthase A (aceB from E. coli), Enzyme: isocitrate lyase (aceA from E. coli), Enzyme: isocitrate dehydrogenase phosphatase/kinase (aceK from E. coli), Enzyme: 3-hydroxy acid dehydrogenase (ydfG from E. coli), Enzyme: acetyl CoA carboxylase (accADBC from E. coli), Polypeptide: predicted transcriptional regulator (yieP from E. coli), Blastocyin resistance gene (BSD from Schizosaccharomyces pombe), Enzyme: pyridine nucleotide transhydrogenase (udha from E. coli), Protein: Cra DNA-binding transcriptional dual regulator (fruR from E. coli), (SCB from E. coli), enzyme: aldehyde dehydrogenase B (aldB from E. coli), Enzyme: carbonic anhydrase (cynT from E. coli), Enzyme: cyanase (cynS from E. coli), DNA gyrase toxin-antitoxin system (ccdAB from E. coli), Enzyme: phosphoglycerate mutase (pgi from E. coli), ArcA transcriptional dual regulator or Aerobic respiration control (arcA from E. coli), Enzyme: 6-phosphofructokinase (pfk from E. coli), Enzyme: glyceraldehyde 3-phosphate dehydrogenase-A complex (gapA from E. coli), aldehyde dehydrogenase A (alda from E. coli), Enzyme: glutamate dehydrogenase (gdhA from E. coli), Enzyme: NADH-dependent serine dehydrogenase (NDSD from Pseudomonas aeruginosa), Protein: threonine/homoserine efflux transporter (rhtA from E. coli), Enzyme: glyceraldehyde 3-phosphate dehydrogenase (gapN from E. coli), Phosphotransferase system (pts from E. coli), Enzyme: 6-phosphofructokinase II (pfkB from E. coli), Enzyme: methylmalonate-semialdehyde dehydrogenase (mmsA from Pseudomonas aeruginosa), Oxaloacetate:beta-alanine aminotransferase (OAT-1 from Bacillus cereus), Enzyme: aspartate 1-decarboxylase (panD from E. coli), Gene that confers resistance to valine (ValR from E. coli), Enzyme: glucokinase (glk from E. coli), Polypeptide: 30 S ribosomal sununit protein S12 (rpsL from E. coli), Polypeptide: CynR DNA-binding transcriptional repressor (cynR from E. coli), Transporter: galactose:H+ symporter (galP from E. coli), aspartate aminotransferase (aspC from E. coli), Enzyme: alpha-ketoglutarate reductase (serA from E. coli), Enzyme: 6-phosphofructokinase I (pfkA from E. coli), Enzyme: phosphoenolpyruvate carboxylase (ppc from E. coli), Enzyme: succinate-semialdehyde dehydrogenase (NADP+) (gabD from E. coli), Enzyme: pyruvate kinase (pyk from E. coli), Enzyme: oxaloacetate 4-decarboxylase (OAD from Leuconostoc mesenteroides), Enzyme: trigger factor; a molecular chaperone involved in cell division (tig from E. coli), Transcription Unit (ptsHlcrr from E. coli), Enzyme: acetyl-CoA acetaldehyde dehydrogenase/alcohol dehydrogenase (adhE from E. coli), Enzyme: fattyacyl thioesterase I (tesA from E. coli), Enzyme: guanosine 3′-diphosphate 5′-triphosphate 3′-diphosphatase (spoT from E. coli), combination of genes encoding accABCD subunits (from E. coli and Halomonas elongata), pol (from E. coli), Enzyme: GDP pyrophosphokinase/GTP pyrophosphokinase (relA from E. coli), [Enzyme Name] (me from E. coli), Enzyme: citrate synthase (gltA from E. coli), Polypeptide: DNA gyrase, subunit A (gyrA from E. coli), Enzyme: multifunctional 2-keto-3-deoxygluconate 6-phosphate aldolase and 2-keto-4-hydroxyglutarate aldolase and oxaloacetate decarboxylase (eda from E. coli), thiamin biosynthesis (thi from E. coli), Polypeptide: acetolactate synthase II (ilvG from E. coli), acetyl CoA carboxylase (accDACB from E. coli), Citrate synthase (ArCS from Arthrobacter aurescens), Acetyl-CoA carboxylase from Corynebacter glutamicum (CgACC from Corynebacter glutamicum), Polypeptide: ferrichrome/phage/antibiotic outer membrane porin FhuA (fhuA from E. coli), Transporter: phosphate:H+ symporter PitA (pitA from E. coli), Transporter: uracil:H+ symporter (uraA from E. coli), Enzyme: uracil phosphoribosyltransferase (upp from E. coli), Enzyme: acylphosphatase (yccX from E. coli), acetyl-CoA synthetase (acsA from E. coli), Polypeptide: restriction of methylated adenine (mrr from E. coli), Protein: TrpR transcriptional repressor (trpR from E. coli), Enzymes: glutamate 5-semialdehyde dehydrogenase/gamma-glutamyl kinase (proAB from E. coli), methylcytosine restriction system (mcrBC from E. coli), Protein: citrate lyase, citrate-ACP transferase component (citF from E. coli), Enzyme: thioesterase II (tesB from E. coli), Enzyme: DNA-specific endonuclease I (endA from E. coli), Enzyme: phosphate acetyltransferase (eutD from E. coli), Enzyme: propionate kinase (tdcD from E. coli), tRNA: tRNA glnV (supE from E. coli), Enzyme: DNA-binding, ATP-dependent protease La (lon from E. coli), Polypeptide: DNA strand exchange and recombination protein with protease and nuclease activity (recA from E. coli), Transcription Unit: restriction endonuclease component of EcoKI restriction-modification system (hsdRMS from E. coli), Enzyme: restriction of DNA at 5-methylcytosine residues (mcrA from E. coli) araD (from E. coli), araB (from E. coli), rhaD (from E. coli), rhaB (from E. coli), ack (from E. coli), fruR (from E. coli), gapA (from E. coli), lad (from E. coli), lacZ (from E. coli), ldhA (from E. coli), mgsA (from E. coli), pfkA (from E. coli), pflB (from E. coli), pgi (from E. coli), poxB (from E. coli), pta-ack (from E. coli), ptsH (from E. coli), glutl (from E. coli) and/or ack (from E. coli) or any combination thereof.

The use of genetic modifications in genetic elements, genes, proteins or the use of compounds, such as siRNA technology, anti-sense technology, and small molecule inhibitors supplied to the host cell that modulate the expression of gene and proteins provided by the present invention are also contemplated.

In some embodiments the genetic modified organism of the invention uses genetic elements such as siRNA ect, genes, proteins or compounds supplied to the host cell to modulate one or more of the following: bifunctional malonyl-CoA reductase (MCR from Chloroflexus aurantiacus), monofunctional malonyl-CoA reductase (caMCR from Chloroflexus aurantiacus), malonyl-CoA reductase (stMCR from Sulfolobus tokodaii), Enzyme: malonyl-CoA reductase (cgMCR from Chloroflexus aggregans), Enzyme: malonyl-CoA reductase (otMCR from Oscillochloris trichoides), Polypeptide: host restriction; endonuclease R (hsdR from E. coli), lactose metabolism (lac from E. coli), L-rhamnulose kinase (rhaB from E. coli), rhamnulose-1-phosphate aldolase (rhaD from E. coli), Enzyme: β-galactosidase (lacZ from E. coli), L-ribulose 5-phosphate 4-epimerase (araD from E. coli), L-ribulokinase (araB from E. coli), Enzyme: D-lactate dehydrogenase—fermentative (ldhA from E. coli), enzyme: pyruvate formate-lyase (pflB from E. coli), Enzyme: phosphate acetyltransferase/phosphate propionyltransferase (pta from E. coli), Enzyme: pyruvate oxidase (poxB from E. coli), Enzyme: methylglyoxal synthase (mgsA from E. coli), enzyme: Acetate kinase (ackA from E. coli), enzymes: phosphotransacetylase-acetate kinase (pta-ack from E. coli), Enzyme: enoyl-[acyl-carrier-protein] reductase (fabI from E. coli), Protein: zeocin binding protein (zeoR from Streptoalloteichus Hindustanus), Enzyme: carboxytransferase moiety of acetyl-CoA carboxylase (accAD from E. coli), Enzyme: triose phosphate isomerase (tpiA from E. coli), Enzyme: biotoin carboxylase moiety of acetyl-CoA carboxylase (accBC from E. coli), Enzyme: transhydrogenase (pntAB from E. coli), Polypeptide: Lad DNA-binding transcriptional repressor (lacI from E. coli), Enzyme: β-ketoacyl-ACP synthases I (fabB from E. coli), Enzyme: β-ketoacyl-ACP synthases II (fabF from E. coli), Enzyme: malonyl-CoA-ACP transacylase (fabD from E. coli), Enzyme: pantothenate kinase (coaA from E. coli), Enzyme: pyruvate dehydrogenase complex (aceEF from E. coli), Enzyme: 3-hydroxyisobutyrate/3-HP dehydrogenase (mmsB from Pseudomonas aeruginosa), Enzyme: lipoamide dehydrogenase (lpd from E. coli), Enzyme: γ-glutamyl-γ-aminobutyraldehyde dehydrogenase (puuC from E. coli), Enzyme: malate synthase A (aceB from E. coli), Enzyme: isocitrate lyase (aceA from E. coli), Enzyme: isocitrate dehydrogenase phosphatase/kinase (aceK from E. coli), Enzyme: 3-hydroxy acid dehydrogenase (ydfG from E. coli), Enzyme: acetyl CoA carboxylase (accADBC from E. coli), Polypeptide: predicted transcriptional regulator (yieP from E. coli), Blastocyin resistance gene (BSD from Schizosaccharomyces pombe), Enzyme: pyridine nucleotide transhydrogenase (udha from E. coli), Protein: Cra DNA-binding transcriptional dual regulator (fruR from E. coli), (SCB from E. coli), enzyme: aldehyde dehydrogenase B (aldB from E. coli), Enzyme: carbonic anhydrase (cynT from E. coli), Enzyme: cyanase (cynS from E. coli), DNA gyrase toxin-antitoxin system (ccdAB from E. coli), Enzyme: phosphoglycerate mutase (pgi from E. coli), ArcA transcriptional dual regulator or Aerobic respiration control (arcA from E. coli), Enzyme: 6-phosphofructokinase (pfk from E. coli), Enzyme: glyceraldehyde 3-phosphate dehydrogenase-A complex (gapA from E. coli), aldehyde dehydrogenase A (alda from E. coli), Enzyme: glutamate dehydrogenase (gdhA from E. coli), Enzyme: NADH-dependent serine dehydrogenase (NDSD from Pseudomonas aeruginosa), Protein: threonine/homoserine efflux transporter (rhtA from E. coli), Enzyme: glyceraldehyde 3-phosphate dehydrogenase (gapN from E. coli), Phosphotransferase system (pts from E. coli), Enzyme: 6-phosphofructokinase II (pfkB from E. coli), Enzyme: methylmalonate-semialdehyde dehydrogenase (mmsA from Pseudomonas aeruginosa), Oxaloacetate:beta-alanine aminotransferase (OAT-1 from Bacillus cereus), Enzyme: aspartate 1-decarboxylase (panD from E. coli), Gene that confers resistance to valine (ValR from E. coli), Enzyme: glucokinase (glk from E. coli), Polypeptide: 30 S ribosomal sununit protein S12 (rpsL from E. coli), Polypeptide: CynR DNA-binding transcriptional repressor (cynR from E. coli), Transporter: galactose:H+ symporter (galP from E. coli), aspartate aminotransferase (aspC from E. coli), Enzyme: alpha-ketoglutarate reductase (serA from E. coli), Enzyme: 6-phosphofructokinase I (pfkA from E. coli), Enzyme: phosphoenolpyruvate carboxylase (ppc from E. coli), Enzyme: succinate-semialdehyde dehydrogenase (NADP+) (gabD from E. coli), Enzyme: pyruvate kinase (pyk from E. coli), Enzyme: oxaloacetate 4-decarboxylase (OAD from Leuconostoc mesenteroides), Enzyme: trigger factor; a molecular chaperone involved in cell division (tig from E. coli), Transcription Unit (ptsHIcrr from E. coli), Enzyme: acetyl-CoA acetaldehyde dehydrogenase/alcohol dehydrogenase (adhE from E. coli), Enzyme: fattyacyl thioesterase I (tesA from E. coli), Enzyme: guanosine 3′-diphosphate 5′-triphosphate 3′-diphosphatase (spoT from E. coli), combination of genes encoding accABCD subunits (from E. coli and Halomonas elongata), pol (from E. coli), Enzyme: GDP pyrophosphokinase/GTP pyrophosphokinase (relA from E. coli), [Enzyme Name] (me from E. coli), Enzyme: citrate synthase (gltA from E. coli), Polypeptide: DNA gyrase, subunit A (gyrA from E. coli), Enzyme: multifunctional 2-keto-3-deoxygluconate 6-phosphate aldolase and 2-keto-4-hydroxyglutarate aldolase and oxaloacetate decarboxylase (eda from E. coli), thiamin biosynthesis (thi from E. coli), Polypeptide: acetolactate synthase II (ilvG from E. coli), acetyl CoA carboxylase (accDACB from E. coli), Citrate synthase (ArCS from Arthrobacter aurescens), Acetyl-CoA carboxylase from Corynebacter glutamicum (CgACC from Corynebacter glutamicum), Polypeptide: ferrichrome/phage/antibiotic outer membrane porin FhuA (fhuA from E. coli), Transporter: phosphate:H+ symporter PitA (pitA from E. coli), Transporter: uracil:H+ symporter (uraA from E. coli), Enzyme: uracil phosphoribosyltransferase (upp from E. coli), Enzyme: acylphosphatase (yccX from E. coli), acetyl-CoA synthetase (acsA from E. coli), Polypeptide: restriction of methylated adenine (mrr from E. coli), Protein: TrpR transcriptional repressor (trpR from E. coli), Enzymes: glutamate 5-semialdehyde dehydrogenase/gamma-glutamyl kinase (proAB from E. coli), methylcytosine restriction system (mcrBC from E. coli), Protein: citrate lyase, citrate-ACP transferase component (citF from E. coli), Enzyme: thioesterase II (tesB from E. coli), Enzyme: DNA-specific endonuclease I (endA from E. coli), Enzyme: phosphate acetyltransferase (eutD from E. coli), Enzyme: propionate kinase (tdcD from E. coli), tRNA: tRNA glnV (supE from E. coli), Enzyme: DNA-binding, ATP-dependent protease La (lon from E. coli), Polypeptide: DNA strand exchange and recombination protein with protease and nuclease activity (recA from E. coli), Transcription Unit: restriction endonulease component of EcoKI restriction-modification system (hsdRMS from E. coli), Enzyme: restriction of DNA at 5-methylcytosine residues (mcrA from E. coli). In some embodiment the genetic modifications listed above are modified further with the genetic modules provided herein.

In some embodiment the genetic modification of the genes, proteins and enzymes of the invention can be for the method of bioproduction of various chemicals which can be used to make various consumer products described herein.

In some embodiment the genetic modification of the genes, proteins and enzymes of the invention can be for the bioproduction of 1,4-butanediol (1,4-BDO) (U.S. Pub. No. 20110190513). In some embodiment the genetic modification of the genes, proteins and enzymes of the invention can be for the bioproduction of butanol (U.S. application Ser. No. 13/057,359). In some embodiment the genetic modification of the genes, proteins and enzymes of the invention can be for the bioproduction of isobutanol (U.S. application Ser. No. 13/057,359)

In some embodiment the genetic modification of the genes, proteins and enzymes of the invention can be for the bioproduction of 3-HP such and its aldehyde metabolites (U.S. application Ser. No. 13/062,917).

In some embodiment the genetic modification of the genes, proteins and enzymes of the invention can be for the bioproduction of polyketide chemical products (U.S. application Ser. No. 13/575,581).

In some embodiment the genetic modification of the genes, proteins and enzymes of the invention can be for the bioproduction of fatty acid methyl esters (U.S. Pub. No. 20110124063). In some embodiment the genetic modification of the genes, proteins and enzymes of the invention can be for the bioproduction of C4-C18 fatty acids (U.S. App No. 61/682,127).

### Genetic Modifications

Various methods to achieve such genetic modification in a host strain are well known to one skilled in the art. Example of genetic modification that can be used by the claimed invention include, but are not limited to, increasing expression of an endogenous genetic element; increasing expression of an exogenous genetic element; decreasing functionality of a repressor gene; increasing functionality of a repressor gene; increasing functionality of a activator gene; decreasing functionality of a activator gene; introducing a genetic change or element integrated in the host genome, introducing a heterologous genetic element permanently, by integration into the genome or transiently by transformation with plasmid; increasing copy number of a nucleic acid sequence encoding a polypeptide catalyzing an enzymatic conversion step; mutating a genetic element to provide a mutated protein to increase specific enzymatic activity; mutating a genetic element to provide a mutated protein to decrease specific enzymatic activity; over-expressing of gene; reduced the expression of a gene; knocking out or deleting a gene; altering or modifying feedback inhibition; providing an enzyme variant comprising one or more of an impaired binding sites or active sites; increasing functionality of a siRNA, decreasing functionality of a siRNA, increasing functionality of a antisense molecule, decreasing functionality of a antisense molecule, addition of genetic modules such as RBS, ′3 UTR elements to increase mRNA stability or translation; deletion of genetic modules such as RBS, ′3 UTR elements to decrease mRNA stability or translation; addition or modification of genetic modules such as ′5 UTR elements to increase transcription; deletion or modification of genetic modules such as ′5 UTR and elements to increase transcription. In addition other genetic modules, provide herein, such a multicopy plasmids and various promoters can be used to further modify of the genetic modifications provide herein. Additionally, as known to those of ordinarily skill in the art compounds such as siRNA technology, anti-sense technology, and small molecule in inhibitors can be used to alter gene expression in the same manner as a genetic modification.

Screening methods, such as SCALE in combination with random mutagenesis may be practiced to provide genetic modifications that provide a benefit to increased production of 3-HP in a host cell. Examples of random mutagenesis can include insertions, deletions and substitutions of one or more nucleic acids in a nucleic acid of interest. In various embodiments a genetic modification results in improved enzymatic specific activity and/or turnover number of an enzyme. Without being limited, changes may be measured by one or more of the following: KM; Kcat, Kavidity, gene expression level, protein expression level, level of a product known to be produced by the enzyme, 3-HP tolerance, or by 3-HP production or by other means.

### Host Cells

In some applications of the invention the host cell can be a gram-negative bacterium. In some applications of the invention the host cell can be from the genera Zymomonas, Escherichia, Pseudomonas, Alcaligenes, or Klebsiella. In some applications of the invention the host cell can be Escherichia coli, Cupriavidus necator, Oligotropha carboxidovorans, or Pseudomonas putida. In some applications of the invention the host cell is one or more an E. coli strains.

In some applications of the invention the host cell can be a gram-positive bacterium. In some applications of the invention the host cell can be from the genera Clostridium, Salmonella, Rhodococcus, Bacillus, Lactobacillus, Enterococcus, Paenibacillus, Arthrobacter, Corynebacterium, or Brevibacterium. In some applications of the invention the host cell is Bacillus licheniformis, Paenibacillus macerans, Rhodococcus erythropolis, Lactobacillus plantarum, Enterococcus faecium, Enterococcus gallinarium, Enterococcus faecalis, or Bacillus subtilis. In some applications of the invention the host cell is B. subtilis strain.

In some applications of the invention the host cell is yeast. In some applications of the invention the host cell can be from the genera Pichia, Candida, Hansenula or Saccharomyces. In some applications of the invention the host cell is Saccharomyces cerevisiae. In some applications of the invention the host cell is Saccharomyces pombe.

In some applications of the invention the host cell is an alga. In some applications of the invention the host cell is a halophile. In some applications of the invention the host cell is an alga. In some applications of the invention the host cell is a chemolithotrophic bacterium.

In some applications of the invention the host cell is comprised of multiple host cell types. In some applications of the invention the host cell is comprised of one host cell type. In some applications of the invention the host cell is comprised of one more species or strain of a host cell type.

### Downstream Consumer Products Chemicals

3-HP purified according to the methods provided in this disclosure may be converted to various other products having industrial uses including, but not limited to, acrylamide, acrylic acid, esters of acrylic acid, 1,3-propanediol, and other chemicals, collectively referred to as “downstream chemical products” or “downstream products.” In some instances the conversion is associated with the separation and/or purification steps. These downstream chemical products are useful for producing a variety of consumer products which are described in more detail below. The methods of the present invention include steps to produce downstream products of 3-HP.

As a C3 building block, 3-HP offers much potential in a variety of chemical conversions to commercially important intermediates, industrial end products, and consumer products. For example, 3-HP may be converted to acrylic acid, acrylates (e.g., acrylic acid salts and esters), 1,3-propanediol, malonic acid, ethyl-3-hydroxypropionate, ethyl ethoxy propionate, propiolactone, acrylamide, or acrylonitrile.

Additionally, 3-HP may be oligomerized or polymerized to form poly(3-hydroxypropionate) homopolymers, or co-polymerized with one or more other monomers to form various co-polymers. Because 3-HP has a single stereoisomer, polymerization of 3-HP is not complicated by the stereo-specificity of monomers during chain growth. This is in contrast to (S)-2-hydroxypropanoic acid (also known as lactic acid), which has two (D, L) stereoisomers that should be considered during its polymerizations.

As will be further described, 3-HP can be converted into derivatives starting (i) substantially as the protonated form of 3-hydroxypropionic acid; (ii) substantially as the deprotonated form, 3-hydroxypropionate; or (iii) as mixtures of the protonated and deprotonated forms. Generally, the fraction of 3-HP present as the acid versus the salt will depend on the pH, the presence of other ionic species in solution, temperature (which changes the equilibrium constant relating the acid and salt forms), and, to some extent, pressure. Many chemical conversions may be carried out from either of the 3-HP forms, and overall process economics will typically dictate the form of 3-HP for downstream conversion.

Acrylic acid obtained from 3-HP purified by the methods described in this disclosure may be further converted to various polymers. For example, the free-radical polymerization of acrylic acid takes place by polymerization methods known to the skilled worker and can be carried out, for example, in an emulsion or suspension in aqueous solution or another solvent. Initiators, such as but not limited to organic peroxides, are often added to aid in the polymerization. Among the classes of organic peroxides that may be used as initiators are diacyls, peroxydicarbonates, monoperoxycarbonates, peroxyketals, peroxyesters, dialkyls, and hydroperoxides. Another class of initiators is azo initiators, which may be used for acrylate polymerization as well as co-polymerization with other monomers. U.S. Pat. Nos. 5,470,928; 5,510,307; 6,709,919; and 7,678,869 teach various approaches to polymerization using a number of initiators, including organic peroxides, azo compounds, and other chemical types, and are incorporated by reference for such teachings as applicable to the polymers described herein.

Accordingly, it is further possible for co-monomers, such as crosslinkers, to be present during the polymerization. The free-radical polymerization of the acrylic acid obtained from dehydration of 3-HP, as produced herein, in at least partly neutralized form and in the presence of crosslinkers is practiced in certain embodiments. This polymerization may result in hydrogels which can then be comminuted, ground and, where appropriate, surface-modified, by known techniques.

An important commercial use of polyacrylic acid is for superabsorbent polymers. This specification hereby incorporates by reference Modern Superabsorbent Polymer Technology, Buchholz and Graham (Editors), Wiley-VCH, 1997, in its entirety for its teachings regarding superabsorbent polymers components, manufacture, properties and uses. Superabsorbent polymers are primarily used as absorbents for water and aqueous solutions for diapers, adult incontinence products, feminine hygiene products, and similar consumer products. In such consumer products, superabsorbent materials can replace traditional absorbent materials such as cloth, cotton, paper wadding, and cellulose fiber. Superabsorbent polymers absorb, and retain under a slight mechanical pressure, up to 25 times or more their weight in liquid. The swollen gel holds the liquid in a solid, rubbery state and prevents the liquid from leaking Superabsorbent polymer particles can be surface-modified to produce a shell structure with the shell being more highly cross-linked than the rest of the particle. This technique improves the balance of absorption, absorption under load, and resistance to gel-blocking. It is recognized that superabsorbent polymers have uses in fields other than consumer products, including agriculture, horticulture, and medicine.

Superabsorbent polymers are prepared from acrylic acid (such as acrylic acid derived from 3-HP provided herein) and a crosslinker, by solution or suspension polymerization. Exemplary methods include those provided in U.S. Pat. Nos. 5,145,906; 5,350,799; 5,342,899; 4,857,610; 4,985,518; 4,708, 997; 5,180,798; 4,666,983; 4,734,478; and 5,331,059, each incorporated by reference for their teachings relating to superabsorbent polymers.

Among consumer products, a diaper, a feminine hygiene product, and an adult incontinence product are made with superabsorbent polymer that itself is made substantially from acrylic acid converted from 3-HP made in accordance with the present invention.

Diapers and other personal hygiene products may be produced that incorporate superabsorbent polymers made from acrylic acid made from 3-HP which is produced and purified by the teachings of the present application. The following provides general guidance for making a diaper that incorporates such superabsorbent polymer. The superabsorbent polymer first is molded into an absorbent pad that may be vacuum formed, and in which other materials, such as a fibrous material (e.g., wood pulp) are added. The absorbent pad then is assembled with sheet(s) of fabric, generally a nonwoven fabric (e.g., made from one or more of nylon, polyester, polyethylene, and polypropylene plastics) to form diapers.

More particularly, in one non-limiting process, multiple pressurized nozzles, located above a conveyer belt, spray superabsorbent polymer particles (e.g., about 400 micron size or larger), fibrous material, and/or a combination of these onto the conveyer belt at designated spaces/intervals. The conveyor belt is perforated and under vacuum from below, so that the sprayed on materials are pulled toward the belt surface to form a flat pad. In various embodiments, fibrous material is applied first on the belt, followed by a mixture of fibrous material and the superabsorbent polymer particles, followed by fibrous material, so that the superabsorbent polymer is concentrated in the middle of the pad. A leveling roller may be used toward the end of the belt path to yield pads of uniform thickness. Each pad thereafter may be further processed, such as to cut it to a proper shape for the diaper, or the pad may be in the form of a long roll sufficient for multiple diapers. Thereafter, the pad is sandwiched between a top sheet and a bottom sheet of fabric (one generally being liquid pervious, the other liquid impervious), for example on a conveyor belt, and these are attached together, for example by gluing, heating or ultrasonic welding, and cut into diaper-sized units (if not previously so cut). Additional features may be provided, such as elastic components, strips of tape, etc., for fit and ease of wearing by a person.

The ratio of the fibrous material to polymer particles is known to affect performance characteristics. In some cases, this ratio is between 75:25 and 90:10 (see e.g., U.S. Pat. No. 4,685,915, incorporated by reference for its teachings of diaper manufacture). Other disposable absorbent articles may be constructed in a similar fashion, such as absorbent articles for adult incontinence, feminine hygiene (sanitary napkins), tampons, etc. (see, for example, U.S. Pat. Nos. 5,009,653; 5,558,656; and 5,827,255 incorporated by reference for their teachings of sanitary napkin manufacture).

Low molecular weight polyacrylic acid has uses for water treatment, and as a flocculant and thickener for various applications including cosmetics and pharmaceutical preparations. For these applications, the polymer may be uncrosslinked or lightly cross-linked, depending on the specific application. The molecular weights are typically from about 200 to about 1,000,000 g/mol. Preparation of these low molecular weight polyacrylic acid polymers is described in U.S. Pat. Nos. 3,904,685; 4,301,266; 2,798,053; and 5,093,472, each of which is incorporated by reference for its teachings relating to methods to produce these polymers.

Acrylic acid may be co-polymerized with one or more other monomers selected from acrylamide, 2-acrylamido-2-methylpropanesulfonic acid, N,N-dimethylacrylamide, N-isopropylacrylamide, methacrylic acid, and methacrylamide, to name a few. The relative reactivities of the monomers affect the microstructure and thus the physical properties of the polymer. Co-monomers may be derived from 3-HP, or otherwise provided, to produce co-polymers. Ullmann's Encyclopedia of Industrial Chemistry, Polyacrylamides and Poly(Acrylic Acids), WileyVCH Verlag GmbH, Wienham (2005), is incorporated by reference herein for its teachings of polymer and co-polymer processing.

Acrylic acid can in principle be copolymerized with almost any free-radically polymerizable monomers including styrene, butadiene, acrylonitrile, acrylic esters, maleic acid, maleic anhydride, vinyl chloride, acrylamide, itaconic acid, and so on. End-use applications typically dictate the co-polymer composition, which influences properties. Acrylic acid also may have a number of optional substitutions and, after such substitutions, may be used as a monomer for polymerization, or co-polymerization reactions. As a general rule, acrylic acid (or one of its co-polymerization monomers) may be substituted by any substituent that does not interfere with the polymerization process, such as alkyl, alkoxy, aryl, heteroaryl, benzyl, vinyl, allyl, hydroxy, epoxy, amide, ethers, esters, ketones, maleimides, succinimides, sulfoxides, glycidyl and silyl (see e.g., U.S. Pat. No. 7,678,869, incorporated by reference above, for further discussion). The following paragraphs provide a few non-limiting examples of copolymerization applications.

Paints that comprise polymers and copolymers of acrylic acid and its esters are in wide use as industrial and consumer products. Aspects of the technology for making such paints can be found in e.g., U.S. Pat. Nos. 3,687,885 and 3,891,591, incorporated by reference for their teachings of such paint manufacture. Generally, acrylic acid and its esters may form homopolymers or copolymers among themselves or with other monomers, such as amides, methacrylates, acrylonitrile, vinyl, styrene and butadiene. A desired mixture of homopolymers and/or copolymers, referred to in the paint industry as “vehicle” (or “binder”) are added to an aqueous solution and agitated sufficiently to form an aqueous dispersion that includes sub-micrometer sized polymer particles. The paint cures by coalescence of these vehicle particles as the water and any other solvent evaporate. Other additives to the aqueous dispersion may include pigment, filler (e.g., calcium carbonate, aluminum silicate), solvent (e.g., acetone, benzol, alcohols, etc., although these are not found in certain no VOC paints), thickener, and additional additives depending on the conditions, applications, intended surfaces, etc. In many paints, the weight percent of the vehicle portion may range from about nine to about 26 percent, but for other paints the weight percent may vary beyond this range.

Acrylic-based polymers are used for many coatings in addition to paints. For example, for paper coating latexes, acrylic acid is used from 0.1-5.0%, along with styrene and butadiene, to enhance binding to the paper and modify rheology, freeze-thaw stability and shear stability. In this context, U.S. Pat. Nos. 3,875,101 and 3,872,037 are incorporated by reference for their teachings regarding such latexes. Acrylate-based polymers also are used in many inks, particularly UV curable printing inks. For water treatment, acrylamide and/or hydroxy ethyl acrylate are commonly co-polymerized with acrylic acid to produce low molecular-weight linear polymers. In this context, U.S. Pat. Nos. 4,431,547 and 4,029,577 are incorporated by reference for their teachings of such polymers. Co-polymers of acrylic acid with maleic acid or itaconic acid are also produced for water-treatment applications, as described in U.S. Pat. No. 5,135,677, incorporated by reference for that teaching. Sodium acrylate (the sodium salt of glacial acrylic acid) can be co-polymerized with acrylamide (which may be derived from acrylic acid via amidation chemistry) to make an anionic co-polymer that is used as a flocculant in water treatment.

For thickening agents, a variety of co-monomers can be used, such as those described in U.S. Pat. Nos. 4,268,641 and 3,915,921, incorporated by reference for their description of these co-monomers. U.S. Pat. No. 5,135,677 describes a number of co-monomers that can be used with acrylic acid to produce water-soluble polymers, and is incorporated by reference for such description.

In some cases, conversion to downstream products may be made enzymatically. For example, 3-HP may be converted to 3-HP-CoA, which then may be converted into polymerized 3-HP with an enzyme having polyhydroxy acid synthase activity (EC 2.3.1.-). Also, 1,3-propanediol can be made using polypeptides having oxidoreductase activity or reductase activity (e.g., enzymes in the EC 1.1.1.- class of enzymes). Alternatively, when creating 1,3-propanediol from 3-HP, a combination of (1) a polypeptide having aldehyde dehydrogenase activity (e.g., an enzyme from the 1.1.1.34 class) and (2) a polypeptide having alcohol dehydrogenase activity (e.g., an enzyme from the 1.1.1.32 class) can be used. Polypeptides having lipase activity may be used to form esters. Enzymatic reactions such as these may be conducted in vitro, such as using cell-free extracts, or in vivo.

Thus, various embodiments described in this disclosure, such as methods of making a chemical, include conversion steps to any downstream products of microbially produced 3-HP, including but not limited to those chemicals described herein, in the incorporated references, and known in the art. For example, in some cases, 3-HP is produced and converted to polymerized-3-HP (poly-3-HP) or acrylic acid. In some cases, 3-HP or acrylic acid can be used to produce polyacrylic acid (polymerized acrylic acid, in various forms), methyl acrylate, acrylamide, acrylonitrile, propiolactone, ethyl 3-HP, malonic acid, 1,3-propanediol, ethyl acrylate, n-butyl acrylate, hydroxypropyl acrylate, hydroxyethyl acrylate, isobutyl acrylate, 2-ethylhexyl acrylate, and acrylic acid or an acrylic acid ester to which an alkyl or aryl addition may be made, and/or to which halogens, aromatic amines or amides, and aromatic hydrocarbons may be added.

a) Reactions that form downstream compounds such as acrylates or acrylamides can be conducted in conjunction with use of suitable stabilizing agents or inhibiting agents reducing the likelihood of polymer formation. See, for example, U.S. Publication No. 2007/0219390, incorporated by reference in its entirety. Stabilizing agents and/or inhibiting agents include, but are not limited to, e.g., phenolic compounds (e.g., dimethoxyphenol (DMP) or alkylated phenolic compounds such as di-tert-butyl phenol), quinones (e.g., t-butyl hydroquinone or the monomethyl ether of hydroquinone (MEHQ)), and/or metallic copper or copper salts (e.g., copper sulfate, copper chloride, or copper acetate) Inhibitors and/or stabilizers can be used individually or in combinations as will be known by those of skill in the art.

In some cases, the one or more downstream compounds are recovered at a molar yield of up to about 100 percent, or a molar yield in the range from about 70 percent to about 90 percent, or a molar yield in the range from about 80 percent to about 100 percent, or a molar yield in the range from about 90 percent to about 100 percent. Such yields may be the result of single-pass (batch or continuous) or iterative separation and purification steps in a particular process.

The methods described in this disclosure can also be used to produce downstream compounds derived from 3-HP, such as but not limited to, polymerized-3-HP (poly-3-HP), acrylic acid, polyacrylic acid (polymerized acrylic acid, in various forms), copolymers of acrylic acid and acrylic esters, acrylamide, acrylonitrile, propiolactone, ethyl 3-HP, malonic acid, and 1,3-propanediol. Also, among esters that are formed are methyl acrylate, ethyl acrylate, n-butyl acrylate, hydroxypropyl acrylate, hydroxyethyl acrylate, isobutyl acrylate, and 2-ethylhexyl acrylate. These and/or other acrylic acid and/or other acrylate esters may be combined, including with other compounds, to form various known acrylic acid-based polymers. Numerous approaches may be employed for such downstream conversions, generally falling into enzymatic, catalytic (chemical conversion process using a catalyst), thermal, and combinations thereof (including some wherein a desired pressure is applied to accelerate a reaction). For example, without being limiting, acrylic acid may be made from 3-HP via a dehydration reaction, methyl acrylate may be made from 3-HP via dehydration and esterification, the latter to add a methyl group (such as using methanol), acrylamide may be made from 3-HP via dehydration and amidation reactions, acrylonitrile may be made via a dehydration reaction and forming a nitrile moiety, propiolactone may be made from 3-HP via a ring-forming internal esterification reaction, ethyl-3-HP may be made from 3-HP via esterification with ethanol, malonic acid may be made from 3-HP via an oxidation reaction, and 1,3-propanediol may be made from 3-HP via a reduction reaction. Additionally, it is appreciated that various derivatives of the derivatives of 3-HP and acrylic acid may be made, such as the various known polymers of acrylic acid and its derivatives. Production of such polymers is considered within the scope of the present invention. Copolymers containing acrylic acid and/or esters have been widely used in the pharmaceutical formulation to achieve extended or sustained release of active ingredients, for example as coating material. Downstream compounds may also be converted to consumer products such as diapers, carpet, paint, and adhesives.

Another important product, acrylamide, has been used in a number of industrial applications. Acrylamide may be produced from 3-HP, for example, without being limiting, via an esterification-amidation-dehydration sequence. Refluxing an alcohol solution of 3-HP in the presence of an acid or Lewis acid catalyst described herein would lead to a 3-HP ester. Treatment of the 3-HP ester with either an ammonia gas or an ammonium ion could yield 3-HP amide. Finally, dehydration of the 3-HP amide with dehydration reagents described elsewhere in this disclosure could produce acrylamide. The steps mentioned herein may be rearranged to produce the same final product acrylamide. Polymerization of acrylamide can be achieved, for example, and without being limiting, by radical polymerization. Polyacrylamide polymers have been widely used as additives for treating municipal drinking water and waste water. In addition, they have found applications in gel electrophoresis, oil-drilling, papermaking, ore processing, and the manufacture of permanent press fabrics.

VIII. Expression Systems General Concepts

The following general concepts are applicable to embodiments of the invention described above.

Multicopy Plasmids

The researcher is faced with a myriad of genetic module options when designing a plasmid for expression of a heterologous protein in a host cell. How to optimize an expression plasmid system often depends on the downstream use of the expressed protein.

Different cloning vectors or plasmids are maintained at different copy numbers, dependent on the replicon of the plasmid. Most general cloning plasmids can carry a DNA insert up to around 15 kb in size.

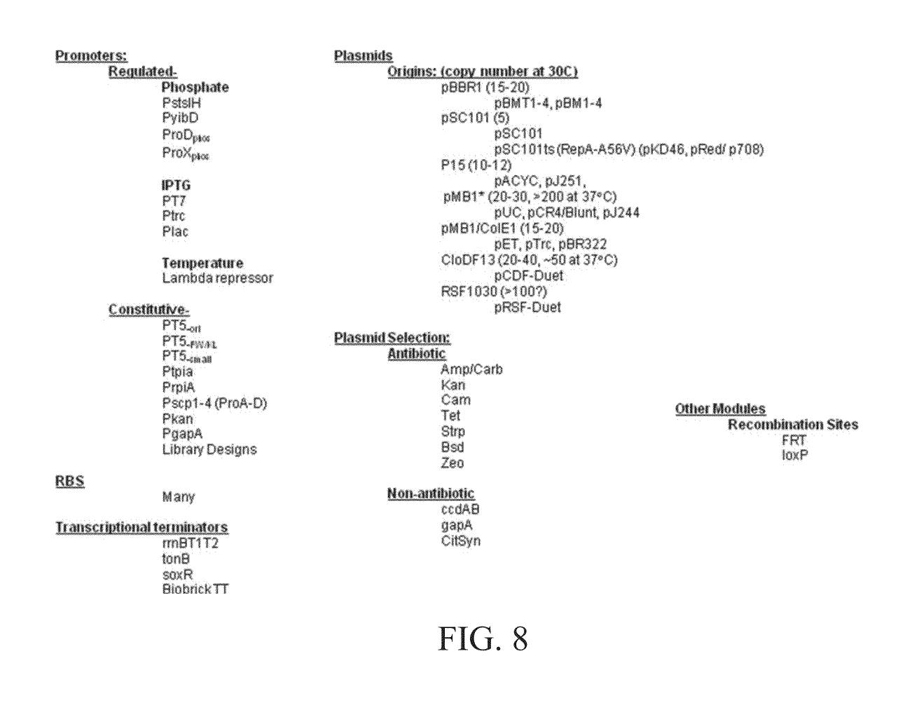

Multicopy plasmids can be used for the expression of recombinant genes in Escherichia coli. Examples of include multicopy plasmids include high-copy, medium-copy and low-copy plasmids (see FIG. 8). The high copy number is generally desired for maximum gene expression. However, the metabolic burden effects can result from multiple plasmid copies could prove to be detrimental for maximum productivity in certain metabolic engineering applications by adding significant metabolic burden to the system.

The low-copy plasmids for example, pBR322 is based on the original ColE1 replicon and thus has a copy number of 15-20. The pACYC series of plasmids are based on the p15A replicon, which has a copy number of 18-22, whereas pSC101 has even a lower copy number around 5, and BACs are maintained at one copy per cell. Such low copy plasmids may be useful in metabolic engineering applications, particularly when one or more of the substrates used in the recombinant pathway are required for normal cellular metabolism and can be toxic to the cell at high levels.

However, the used of high-copy plasmids may be useful in enhanced cellular metabolism contexts. The mutant ColE1 replicon, as found in the pUC series of plasmids produces a copy number of 500-700 as a result of a point mutation within the RNAII regulatory molecule.

There are transcription and translation vectors. Transcription vectors are utilized when the DNA to be cloned has an ATG start codon and a prokaryotic ribosome-binding site. Translation vectors contain an efficient ribosome-binding site and, therefore, it is not necessary for the target DNA to contain one. This is particularly useful in cases where the initial portion of the gene may be cleaved in an effort to improve solubility. Another consideration when choosing a transcription or translation vector is the source of the DNA to be expressed. Prokaryotic genes usually have a ribosome-binding site that is compatible with the host E. coli translation machinery, whereas eukaryotic genes do not. Normal prokaryotic gene expression may be enhanced by use of an engineered promoter and ribosome-binding site.

Promoters

A promoter is a region of DNA that initiates transcription of a particular gene. In bacteria, transcription is initiated by the promoter being recognized by RNA polymerase and an associated sigma factor, which are often brought to the promoter site by an activator protein's binding to its own DNA binding site located by the promoter.

Promoter selection is an important factor when designing an expression plasmid system. A promoter is located upstream of the ribosome-binding site. Owing to the fact that many heterologous protein products are toxic to the cell, the promoter can be regulated so that the heterologous protein is expressed at the appropriate amount and time to reduced the burden on the cell host.

Historically, the most commonly used promoters have been the lactose (lac) and tryptophan (trp) promoters. These two promoters were combined to create the hybrid promoters tac and trc that are also commonly used. Other common promoters are the phage lambda promoters, the phage T7 promoter (T7), and the alkaline phosphatase promoter (phoA).

Promoters can be constitutive and inducible. Constitutive promoter is active in all circumstances in the cell, while regulated or inducible promoter become active in response to specific stimuli. In addition the strength of the promoter can also differ. A strong promoter has a high frequency of transcription and generates the heterologous protein as 10-30% of the total cellular protein production (for examples see FIG. 8). Chemically-inducible promoters that can be used in various aspects of the invention include but are not limited to promoters whose transcriptional activity is regulated by the presence or absence of alcohol, tetracycline, steroids, metal and other compounds. Physically-inducible promoters that can be used in various aspects of the invention include but are not limited to including promoters whose transcriptional activity is regulated by the presence or absence of light and low or high temperatures.

In order to be an inducible promoter, the promoter should be initially be completely repressed to transcription and then transcription induced with the addition of an inducer to allow expression at the time that expression is desired in the host cell. Alternatively, an inducible promoter may be responsive to the lack of a substance, such as inorganic phosphate, such that the absence of inorganic phosphate will allow expression at the time that expression is desired in the host cell (for examples see FIG. 8).

Ribosome Binding Sites

A Ribosome Binding Sites (RBS) is an RNA sequence upstream of the start codon that affects the rate at which a particular gene or open reading frame (ORF) is translated. One can tailor an RBS site to a particular gene. Ribosome Binding Sites (RBSs) are typically short sequences, often less than 20 base pairs. Various aspects of RBS design are known to affect the rate at which the gene is translated in the cell. The RBS module can influences the translation rate of a gene largely by two known mechanisms. First, the rate at which ribosomes are recruited to the mRNA and initiate translation is dependent on the sequence of the RBS. Secondly, the sequence of the RBS can also affect the stability of the mRNA in the cell, which in turn affects the number of proteins. Through the use of genetic expression modules the expression of desired genes, such as genes encoding enzymes in the biosynthetic pathway for 3-HP, can be tailored activity either at the transcriptional and translational level.

One can access the registry RBS collection as a starting point for designing an RBS <<http://partsregistry.org/Ribosome_Binding_Sites/Catalog>>. The Registry has collections of RBSs that are recommended for general protein expression in E. coli and other prokaryotic hosts. In addition, each family of RBSs has multiple members covering a range of translation initiation rates. There are also several consensus RBS sequence for E. coli have been described. However, it is important to keep in mind the known RBS functions and mechanisms in a larger context. For example, in certain contexts the RBS can interact with upstream sequences, such as sequence that comprise the promoter or an upstream ORF. In other contexts, the RBS may interact with downstream sequences, for example the ribosome enzyme binds jointly to the RBS and start codon at about the same time. These potential interactions should be considered in the overall RBS sequence design. The degree of secondary structure near the RBS can affect the translation initiation rate. This fact can be used to produce regulated translation initiation rates.

The Shine-Dalgarno portion of the RBS is critical to the strength of the RBS. The Shine-Dalgarno is found at the end of the 16s rRNA and is the portion that binds with the mRNA and includes the sequence 5′-ACCUCC-3′. RBSs will commonly include a portion of the Shine-Dalgarno sequence. One of the ribosomal proteins, S1, is known to bind to adenine bases upstream from the Shine-Dalgarno sequence. As a result, the RBS can be made stronger by adding more adenines in the sequence upstream of the RBS.

When considering the design of the spacing between the RBS and the start codon, it is important to think of the aligned spacing rather than just the absolute spacing. While the Shine-Dalgarno portion of the RBS is critical to the strength of the RBS, the sequence upstream of the Shine-Dalgarno sequence is also important. Note that the promoter may add some bases onto the start of the mRNA that may affect the strength of the RBS by affecting S1 binding. Computer programs that design RBS sequence to match protein coding sequences, desired upstream sequences including regulatory mRNA sequences, and account of secondary structure are known [Salis, Mirsky, and Voight, Nature Biotechnology 27: 946-950, 2009] and were used to optimize RBSs for the ACCase subunit genes as described in (see EXAMPLE 3).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

### Example 1

### Salt Inhibition Studies in E. coli

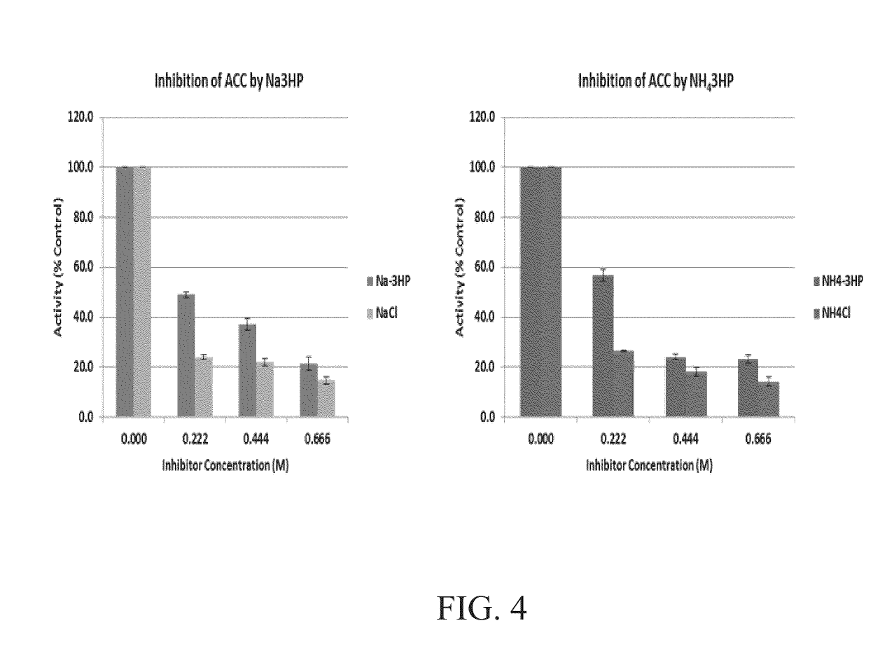

The activity of ACCase complex, a critical enzyme in the conversion of acetyl-CoA to malonyl-CoA, the immediate precursor for 3-HP, is severely inhibited by salt. Dose-dependent effects on ACCase activity was observed in the presence of NaCl, NH₄Cl, Na-3-HP, or NH₄-3-HP such that salt levels near 0.44M resulted in decreasing the activity of the ACCase enzyme by approximately 80%, while salts of 3-HP levels near 0.66M decreased the activity of the ACCase enzyme by approximately 80% relative to control (FIG. 4). Levels of greater than 0.66M (60 g/L) are expected to be present for commercially viable commercial production of 3-HP.

### Example 2

### ACCase from Halophilic Organism

Halophilic organisms, such as Halomonas elongata, are found in environments with high salt concentrations and, in general, have a salt internal concentration >2.5-3M. It is hypothesized that enzymes derived from any salt-tolerant species should be more resistant to enzyme inhibition by salts, such as 3-HP. Further, these enzymes that have greater salt tolerance should in turn have extended production under high salt conditions than enzymes with lower salt tolerance.

Accordingly, the genes encoding the accA, accB, accC, accD of H. elongata described in Table 1 were synthesized for expression in E. coli using codons optimized for this organism and supplied individually on pUC57 plasmids without promoters. Synthetic operons comprising the subunits were assembled using the Gibson assembly method.

TABLE 1
Accession numbers for genes encoding ACCase subunits
from Halomonas elongata

| Gene | Accession number | SEQ ID NO. |
|---|---|---|
| accA | YP_003898857.1 | SEQ ID NO. 1, 2 |
| accB | YP_003897250.1 | SEQ ID NO. 3, 4 |
| accC | YP_003897249.1 | SEQ ID NO. 5, 6 |
| accD | YP_003897309.1 | SEQ ID NO. 7, 8 |

Each gene was amplified by PCRs with Pfu Ultra II HS using the manufacturer's instructions, and the PCR products were purified using the Zymo PCR Cleanup kit. Concentrations of products were measured using the Nanodrop spectophotometer. The Gibson Assembly kit (NEB) was used to construct plasmids expressing the ACCase subunit genes as directed by the manufacturer. The effect of NH₄-3-HP and NH₄Cl on H. elongata ACCase was tested and compared to the E. coli ACCase. As shown in FIG. 4, whereas the E. coli ACCase is significantly inhibited by the salts, the ACCase from the halophile is less affected by either NH₄-3-HP or by NH₄Cl. This result indicated that use of the H. elongata ACCase in 3-HP production strains would in beneficial in relieving 3-HP inhibition of the conversion of acetyl-CoA to malonyl-CoA, a critical step in the pathway.

### Example 3

### RBS-Optimized Genes

Enzyme expression is regulated at transcriptional and translational levels in prokaryotes. Ribosome Binding Sites (RBS) are 15 nucleotide segments which are known to control the level of protein expression in microorganisms. To enhance H. elongata ACCase expression various customized RBS were constructed and optimized for E. coli translation expression. Table 2 shows the RBS sequences used to increase expression of the individual subunits.

TABLE 2
Table 2. RBS sequences used to enhance expression of
H. elongate ACCase subunits.

| H. elongata | | | | |
|---|---|---|---|---|
| ACC | | | | |
| expression | Modified RBS sequences preceeding ATG (underlined) | | | |
| plasmid | He_accD | He_accA | He_accC | He_accB |
| Parent 2-4 | 5′- | 5′- | 5′- | 5′- |
| | GCGTAGTAAAGGA | CAATTTATTTAAGGA | GAAATTTCATACC | GGAAGAACAAGGG |
| | GGTAACATATG | GGACTCTTAAGATG | ACAGGCGAAGGAG | GTGTACATG |
| | | | GAAAAACCATG | |
| B2 | Same as 2-4 | Same as 2-4 | Same as 2-4 | 5′- |
| | | | | ggaagaattaagg |
| | | | | gggacaaggggga |
| | | | | ataATG |
| 13A | 5′- | Same as 2-4 | Same as 2-4 | |
| | gcgtagtagccggg | | | |
| | tgataaggagccgt | | | |
| | aacATG | | | |
| 14C | 5′- | Same as 2-4 | Same as 2-4 | Same as 2-4 |
| | gcgtagtagctgat | | | |
| | ataaaaggaggtaa | | | |
| | cggATG | | | |
| 15C | Same as 2-4 | 5′- | Same as 2-4 | Same as 2-4 |
| | | caatttatttttgtt | | |
| | | cacccaaggagtatt | | |
| | | gctaATG | | |
| 17C | Same as 2-4 | 5′- | Same as 2-4 | Same as 2-4 |
| | | caatttatttaccga | | |
| | | aataaaaggagggat | | |
| | | gcgaATG | | |
| 35C | 5′- | 5′- | Same as 2-4 | Same as 2-4 |
| | gcgtagtagccggg | caatttatttttgtt | | |
| | tgataaggagccgt | cacccaaggagtatt | | |
| | aacATG | gctaATG | | |
| 36C | 5′- | 5′- | Same as 2-4 | Same as 2-4 |
| | gcgtagtagccggg | caatttatttaccga | | |
| | tgataaggagccgt | aataaaaggagggat | | |
| | aacATG | gcgaATG | | |
| 36C-8 | 5′- | 5′- | Same as 2-4 | 5′- |
| | gcgtagtagccggg | caatttatttaccga | | ggaagaattaagg |
| | tgataaggagccgt | aataaaaggagggat | | gggacaaggggga |
| | aacATG | gcgaATG | | ataATG |
| 72B | 5′- | 5′- | 5′- | 5′- |
| | gcgtagtagccggg | caatttatttaccga | TCTTCCCACAACA | GAAATTTCATACC |
| | tgataaggagccgt | aataaaaggagggat | CTGGCGGACTCCA | ACAGGCGAAGGAG |
| | aacATG | gcgaATG | TCATG | GAAAAACCATG |
| 105F | 5′- | 5′- | 5′- | 5′- |
| | gcgtagtagccggg | caatttatttttgtt | TCTTCCCACAACA | GAAATTTCATACC |
| | tgataaggagccgt | cacccaaggagtatt | CTGGCGGACTCCA | ACAGGCGAAGGAG |
| | aacATG | gctaATG | TCATG | GAAAAACCATG |

The expression performance of the RBS-optimized H. elongata ACCases was evaluated by 3-HP production in a 96-well format, each in triplicate wells, and the averaged results shown in Table 3. Specific 3HP production is shown as g/L per OD₆₀₀. As may be seen in Table 3, enhancing the efficiency of the RBS in strains B2, 35C, and 72 B clearly resulted in increased malonyl-CoA production leading to increased 3-HP production. It is evident from these results that combinations of enhanced RBS's before each of the individual genes accA, accB, accC, and accD may result in strains with even higher ACCase expression and activity.

TABLE 3
Improvement in 3-HP production by RBS-optimized expression
of H. elongata ACCase subunits.

| H. elongata ACCase | 3HP |
|---|---|
| expression plasmid | (g/I.OD) |
| Parent 2-4 | 0.06 |
| B2 | 0.81 |
| 13A | 0.01 |
| 14C | 0.54 |
| 15C | 0.14 |
| 17C | 0.08 |
| 35C | 0.68 |
| 36C | 0.31 |
| 36C-8 | 0.31 |
| 72B | 0.57 |
| 105F | 0.19 |

### Example 4

### Coordinated Expression by Subunit Fusions

In nature ACCase subunit genes from prokaryotes such as E. coli and H. elongata have been shown to have a quaternary structure: accA₂:accD₂:accB₄:accC₂. However, the intrinsic levels of the ACCase subunit genes are too low for optimal production. Therefore, for optimal production it is ideal to have overexpression to be coordinated in a similar manner.

Expression of the genes encoding each ACCase subunit is regulated at transcriptional and translational levels. Coordinated overexpression of the ACCase subunit genes, accA, accB, accC, accD should give better ACCase activity. Examples of fusions of accC-B proteins exist in bacteria. It is hypothesized that coordinated overexpression may be achieved by fusion of subunit genes should ensures equimolar expression of the subunit genes at the optimal time.

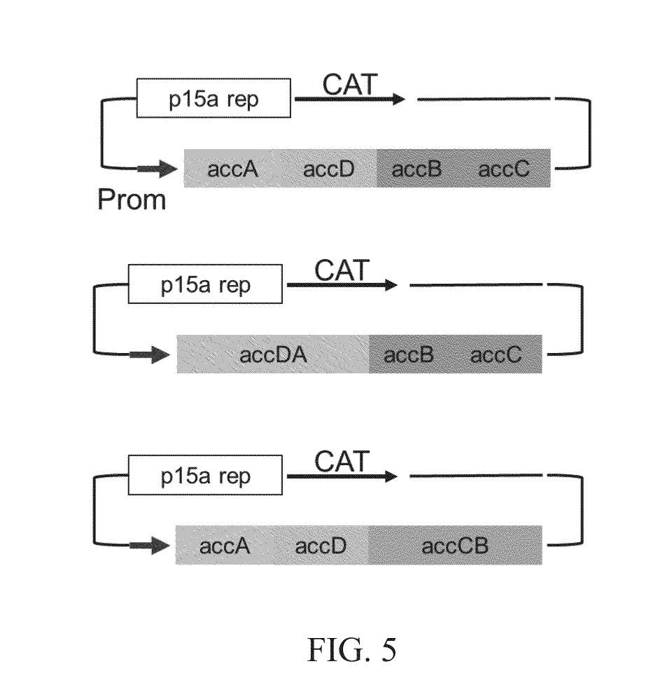

The following ACCase subunit gene fusion were constructed and the constructs overexpressed in E. coli: (A) Control ABCD, (B) fusion of accC-B (SEQ ID NO. s 9, 10) subunit genes as seen in bacteria, (C) fusion of accD-A subunit genes using a flexible 15-amino acid linker (Linker sequence LSGGGGSGGGGSGGGGSGGGGSAAA; SEQ ID NO. s 11, 12) as depicted in FIG. 5.

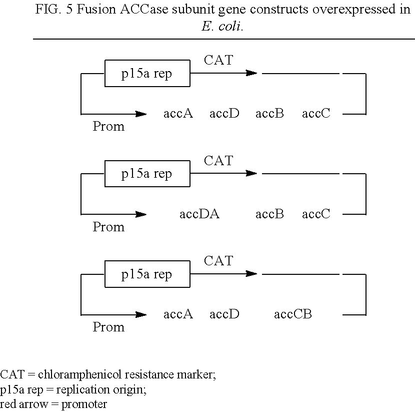

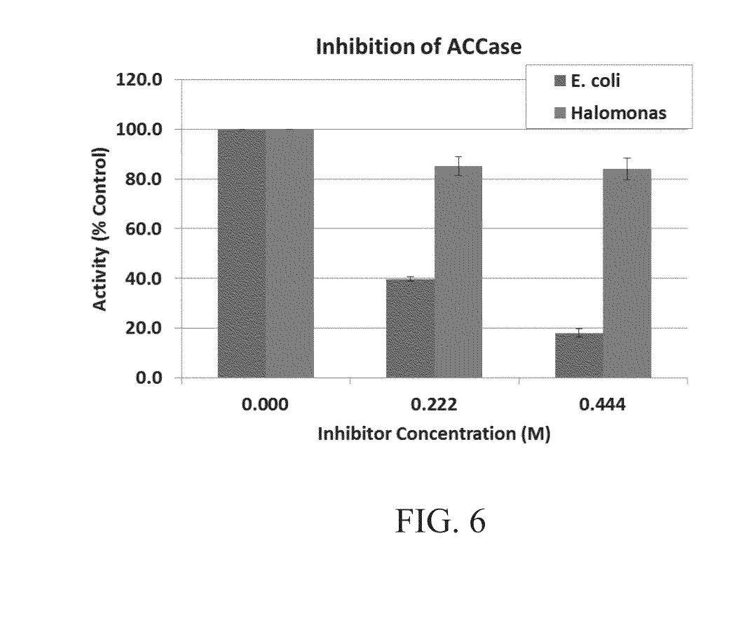

The performance of the ACC fusions were tested for their ACCase activity and for various 3-HP production metrics in Table 4. ACCase activity was determined in cell lysates using an assay for malonyl-CoA production as described in [Kroeger, Zarzycki, and Fuchs, Analytical Biochem. 411:100-105, 2011]. Production of 3-HP was determined in cells co-transformed with a plasmid bearing the genes encoding the malonyl-CoA reductase from S. tokadaii and E. coli ydfG providing a 3-HP dehydrogenase to complete the metabolic pathway from malonyl-CoA to 3HP. These results show that the strain with the fused accDA genes had higher average specific productivity of 3-HP compared to the parental strain in which the overexpressed ACCase is not fused. FIG. 6 shows that the benefit of the accDA fusion were also manifested in 3-HP production in fermentors with environmental controls of nutrient feed, pH, aeration, and temperature.

TABLE 4
Table 4: ACC Fusions and ACCase activity

| | | Avg specific | Avg specific | |
|---|---|---|---|---|
| | | prodn rate | prodn rate | ACCase specific |
| Strain | | (g/gDCW · h) at | (g/gDCW · hr) at | activity at TS + 6 |
| designation | Plasmid | TS + 6 | TS + 20 | (U/mg) |
| BX3_783 | Parent (unfused ACCase) | 0.160 | 0.146 | 0.057 |
| BX3_829 | No ACC | 0.069 | 0.062 | 0.000 |
| BX3_837 | EC ACC DA fusion | 0.209 | 0.201 | 0.054 |

### Example 5

### 3-HP Exporter

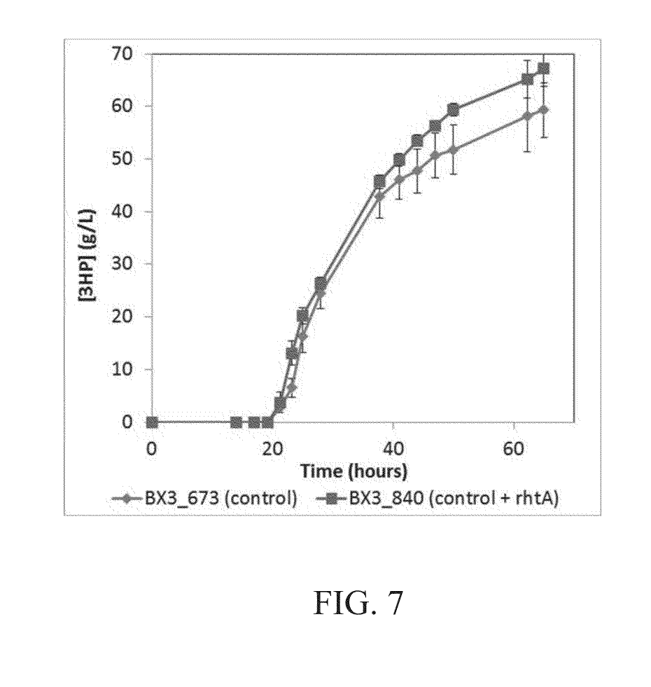

Growth inhibition has been demonstrated for E. coli strains grown in the presence of 3-HP at levels as low as 20 g/L. To produce high titers of 3-HP the production host is required to balance production with overcoming inhibition. A known chemical exporter from E. coli that has been previously characterized for homoserine transport, rhtA, was evaluated for increased production of 3-HP. A mutant version of the exporter, rhtA(P2S) (SEQ ID NO. 30 nucleic acid, SEQ ID NO. 31 protein) was synthesized behind the PtpiA promoter and inserted into the pTRC-PyibD-MCR plasmid behind a terminator using the Gibson Assembly kit (NEB) according to manufacturer's instructions. The effects of overexpression of rhtA were evaluated in 1 L fermentation compared to the control plasmid without rhtA. As shown in FIG. 7, overexpression of rhtA resulted in a significant improvement in 3HP titer compared to the control production strain. Construction of plasmids expressing another putative transporter, ydcO (SEQ ID NO. 32 nucleic acid, SEQ ID NO. 33 protein) is carried out in the same manner.

### Example 6

### Bicarbonate Importer (Prophetic)

Increased import of bicarbonate to increase availability of bicarbonate for the ACCase reaction will increase production of malonyl-CoA and hence products derived metabolically from malonyl-CoA, such as 3-HP. The gene encoding the bicA bicarbonate transporter (SEQ ID NO. 13) of Synechococcus sp. was synthesized using codons optimized for expression in E. coli (SEQ ID NO. 14) and expressed using the E. coli tal promoter in a strain cotransformed with plasmids encoding ACCase and MCR functions. Production of 3-HP by this strain is compared to that achieved by a control strain without overexpressed bicA.

What is claimed is:

1. A genetically modified organism capable of producing a chemical product of interest, wherein the genetic modification includes introduction of nucleic acid sequences coding for polynucleotides encoding a malonyl-CoA reductase gene that is mutated such that the encoded malonyl CoA reductase enzyme has enhanced activity at lower temperatures.

2. The genetically modified organism of claim 1 wherein the mutated malonyl-CoA reductase enzyme has enhanced activity at about 20° C. to about 44° C.

3. The genetically modified organism of claim 1 wherein the mutated malonyl-CoA reductase enzyme has enhanced activity at about 30° C. to about 37° C.

4. The genetically modified organism of claim 1 wherein the mutated malonyl-CoA reductase enzyme has enhanced activity at about 32° C. to about 38° C.

5. The genetically modified organism of claim 1, 2, 3, or 4 wherein the mutated malonyl-CoA reductase enzyme is derived from Sulfolobus tokodaii.

6. A genetically modified organism capable of producing a chemical product of interest, wherein the genetic modification includes introduction of nucleic acid sequences coding for polynucleotides encoding salt-tolerant enzymes.

7. The genetically modified organism of claim 6 wherein said polynucleotides encode for one or more enzymes from salt-tolerant organisms that are homologs of a key enzyme selected from the group consisting of: Sucrose-6-phosphate hydrolase (cscA from E. coli), glucose-6-phosphate isomerase (pgi from E. coli), fructokinase (cscK from E. coli), fructose-1,6-bisphosphatase (yggF from E. coli), fructose 1,6-bisphosphatase (ybhA from E. coli), fructose 1,6-bisphosphatase II (glpX from E. coli), fructose-1,6-bisphosphatase monomer (fbp from E. coli), 6-phosphofructokinase-1 monomer (pfkA from E. coli), 6-phosphofructokinase-2 monomer (pfkB from E. coli), fructose bisphosphate aldolase monomer (fbaB from E. coli), fructose bisphosphate aldolase monomer (fbaA from E. coli), triose phosphate isomerase monomer (tpiA), glyceraldehyde 3-phosphate dehydrogenase-A monomer (gapA from E. coli), phosphoglycerate kinase (pgk), 2,3-bisphosphoglycerate-independent phosphoglycerate mutase (gpmM from E. coli), 2,3-bisphosphoglycerate-dependent or tdcE (from E. coli), phosphoglycerate mutase (gpmA), enolase (eno from E. coli), phosphoenolpyruvate carboxylase (ppc from E. coli), malate dehydrogenase (mdh), fumarase A (fum from E. coli), fumarase B (fumB), fumarase C (fumC from E. coli), phosphoenolpyruvate synthetase (ppsA from E. coli), pyruvate kinase I monomer (pykF from E. coli), pyruvate kinase II monomer (pykA from E. coli), fumarate reductase (frdABCD from E. coli), lipoamide dehydrogenase (lpd from E. coli), pyruvate dehydrogenase (aceE from E. coli), pyruvate dehydrogenase (aceF from E. coli), pyruvate formate-lyase (pflB from E. coli), acetyl-CoA carboxylase (accABCD from E. coli), malonyl CoA reductase (mcr), 3HP dehydrogenase (mmsB, NDSD, or ydfG), and malonate semialdehyde reductase (nemA, rutE from E. coli).

8. The genetically modified organism of claim 7 wherein said homologs include enzymes that have at least 99%, 98%, 97%, 96%, 95%, 94%, 93%, 92%, 91%, 90%, 89%, 88%, 87%, 86%, 85%, 84%, 83%, 82%, 81% or 80% overall amino acid or nucleotide identity to said key enzyme.

9. The genetically modified organism of claim 7 wherein said homologs include enzymes that have at least 99%, 98%, 97%, 96%, 95%, 94%, 93%, 92%, 91%, 90%, 89%, 88%, 87%, 86%, 85%, 84%, 83%, 82%, 81% or 80% overall amino acid or nucleotide identity to the essential protein function domains of said key enzyme.

10. The genetically modified organism of claim 7 wherein said homologs include enzymes that have at least 99%, 98%, 97%, 96%, 95%, 94%, 93%, 92%, 91%, 90%, 89%, 88%, 87%, 86%, 85%, 84%, 83%, 82%, 81% or 80% overall amino acid or nucleotide identity to the essential binding amino acids within an essential protein function domain of said key enzyme.

11. A genetically modified non-salt-tolerant organism capable of producing a chemical product of interest, wherein the genetic modification includes introduction of nucleic acid sequences coding for polynucleotides encoding an acetyl-CoA carboxylase from a salt-tolerant organism.

12. The genetically modified organism of claim 7, or 11 wherein said salt-tolerant organism is selected from the group consisting of Halomonas elongata, Salinibacter rubur, and Halobacterium species (Archaea).

13. A genetically modified organism capable of producing a chemical product of interest, wherein the genetic modification includes introduction of nucleic acid sequences coding for polynucleotides encoding a gene that codes for a protein that facilitates the exportation of a chemical product of interest or the export of an inhibitory chemical from within the cell to the extracellular media.

14. The genetically modified organism of claim 13 wherein said gene is within a gene family selected from the group consisting of: major facilitator superfamily (MFS), ATP-binding cassette superfamily (ABC), small multidrug resistance family (SMR), resistance-nodulation-cell division superfamily (RND), and multi antimicrobial extrusion protein family (MATE).

15. The genetically modified organism of claim 13 wherein said gene is a solvent tolerant transporter.

16. The genetically modified organism of claim 15 wherein said solvent tolerant transporter transports a chemical selected from the group consisting of: bromoacetate, butanol, and isobutanol.

17. The genetically modified organism of claim 13 wherein said gene is selected from the group consisting of acrD, bcr, cusA, dedA, eamA, eamB, eamH, emaA, emaB, emrB, emrD, emrKY, emrY, garP, gudP, hsrA, leuE, mdlB, mdtD, mdtG, mdtL, mdtM, mhpT, rhtA, rhtB, rhtC, thtB, yahN, yajR, ybbP, ybiF, ybjJ, ycaP, ydcO, yddG, ydeD, ydhC, ydhP, ydjE, ydjI, ydjK, yeaS, yedA, yeeO, yegH, yfcJ, yfiK, yhjE, yidE, yiJE, yjil, yjiJ, yjiO, ykgH, ypjD, ytfF, and ytfL homologs.

18. The genetically modified organism of claim 13 that is encoded for more than one exporter gene.

19. The genetically modified organism of claim 13 wherein a tig gene is deleted from the genome of the organism.

20. A genetically modified organism capable of producing a chemical product of interest, wherein the genetic modification includes introduction of nucleic acid sequences coding for polynucleotides encoding a gene that encodes a protein that (1) facilitates the importation from the extracellular media to within the cell of a reactant, precursor, or metabolite used in the organism's production pathway for producing a chemical product of interest; or (2) inhibits the exportation of said reactant, precursor, or metabolite from within the cell membrane to the media.

21. The genetically modified organism of claim 20 wherein the reactant, precursor or metabolite is carbon dioxide or bicarbonate.

22. The genetically modified organism of claim 21 wherein said gene is selected from the group consisting of bicA from Synechococcus species, ychM gene product of E. coli, and yidE gene product of E. coli.

23. A method of producing 3-HP using the genetically modified organisms of claim 1, 6, 13, or 20, wherein the organism's reaction pathway converts sugar to malonyl-CoA through a series of intermediate steps, converts malonyl-CoA to malonate semialdehyde, and converts malonate semialdehyde to 3-HP.

24. A method for producing a chemical for a consumer product, said method comprising:
combining a carbon source and a genetically modified host cell;
culturing said genetically modified host cell under suitable conditions to sustain continuous cell proliferation of said host cell, wherein said genetically modified host cell has enhanced conversion of said carbon source to a chemical for a consumer product.

25. The method of claim 24, wherein said method further comprises culturing said genetically modified host cell under suitable conditions to sustain continuous cell proliferation of said host cell.

26. The method of claim 24, wherein said genetically modified host cell is further modified for increased conversion of said carbon source to acetyl-CoA.

27. The method of claim 24, wherein said genetically modified host cell is further modified for increased conversion of said carbon source to malonyl-CoA.

28. The method of claim 24, wherein said genetically modified host cell is further modified for increased conversion of said carbon source to acetyl-CoA and malonyl-CoA.

29. The method of claim 26, wherein said further modification comprises a decrease in production of ethanol, lactate, acetate production or combination thereof in said host cell.

30. The method of claim 24, wherein said genetically modified host cell is further modified with an inducible gene that shuts off the flux of malonyl-CoA reductase to the fatty acid production pathway.

31. The method of claim 24, wherein said genetically modified host cell is further modified with an inducible gene that shuts off the flux of malonyl-CoA reductase to the fatty acid production pathway and decreases ethanol, lactate, acetate production or combination thereof in said host cell.

32. The method of claim 31, wherein said genetically modified host cell is further modified with an inducible genetic module that shuts off aceEF, lpd, pta, ackA, adhE, fabI, fabB, or fabD, or combinations thereof.

33. The method of claim 32, wherein said inducible genetic module is induced by chemical or temperature induction.

34. The method of claim 24, wherein said genetically modified host cell is further modified with malonyl-CoA reductase enzyme that has a dehydrogenase domain that enhances malonate semialdehyde or 3-HP production.

35. The method of claim 34, wherein said malonyl-CoA reductase enzyme is monofunctional.

36. The method of claim 35, wherein said monofunctional malonyl-CoA reductase enzyme has at least 90% identity to the malonyl-CoA reductase derived from Sulfolobus tokodaii, Chloroflexus aggregans, or Oscillochloris trichoides.

37. The method of claim 36, wherein said monofunctional malonyl-CoA reductase is further comprised of an inducible genetic module.

38. The method of claim 37, wherein said inducible genetic module is induced by chemical or temperature induction.

39. The method of claim 34, wherein said malonyl-CoA reductase enzyme is bifunctional.

40. The method of claim 39, wherein said bifunctional malonyl-CoA reductase enzyme has at least 90% identity to the malonyl-CoA reductase derived from Chloroflexus aurantiacus.

41. The method of claim 39, wherein said bifunctional malonyl-CoA reductase is further comprised of an inducible genetic module.

42. The method of claim 41, wherein said inducible genetic module is induced by chemical or temperature induction.

43. The method of claim 34, wherein said dehydrogenase domain enhances said malonyl-CoA reductase enzyme to be less reversible or irreversible.

44. The method of claim 43, wherein said dehydrogenase domain is rutE or nemA.

45. The method of claim 34, wherein said dehydrogenase domain use a redundant other than NADPH.

46. The method of claim 45, wherein said dehydrogenase domain use the NADH redundant.

47. The method of claim 46, wherein said dehydrogenase domain is mmsB.

48. The method of claim 45, wherein said dehydrogenase domain uses the falvin redundant.

49. The method of claim 48, wherein said dehydrogenase domain is rutE and nemA.

50. The method of claim 43, wherein said dehydrogenase domain enhances said malonyl-CoA reductase enzyme to be more tolerant to 3-hydroxypropionic acid inhibition.

51. The method of claim 50, wherein said dehydrogenase domain is ydfG or NAD+-dependent serine dehydrogenase.

52. (canceled)

53. (canceled)

54. The method of claim 24, wherein said genetically modified host cell further comprises acetyl-CoA carboxylase.

55. (canceled)

56. The method of claim 24, wherein said genetically modified host cell further comprises malonyl-CoA reductase enzyme derived from a halophilic organism.

57. The method of claim 56, wherein said halophilic organism is from the genera Halomonase.

58. The method of claim 24, wherein said genetically modified host cell is further modified with the genes encoding at least one protein that has at least 90% homology of Table 2.

59. The method of claim 24, wherein said genetically modified host cell is further modified with at least one efflux transporter, capable of exporting 3-HP ions.

60. The method of claim 59, wherein said efflux transporter, is selected from the group: MFS exporter, ABC exporter, SMR exporter, RND exporter, MATE exporter, amino acid exporters, and solvent tolerance transporters or a combination thereof.

61. The method of claim 59, wherein said efflux transporter is selected from the group: acrD, bcr, cusA, dedA, eamA, eamB, eamH, emaA, emaB, emrB, emrD, emrKY, emrY, garP, gudP, hsrA, leuE, mdlB, mdtD, mdtG, mdtL, mdtM, mhpT, rhtA, rhtB, rhtC, thtB, yahN, yajR, ybbP, ybiF, ybjJ, ycaP, ydcO, yddG, ydeD, ydgE, yddG, ydhC, ydhP, ydiN, ydiM, ydjE, ydjI, ydjK, yeaS, yedA, yeeO, yegH, yggA, yfcJ, yfiK, yhjE, yidE, yigK, yigJ, yijE, yjil, yjiJ, yjiO, ykgH, ypjD, ytfF, ytfL or homologs thereof.

62. The method of claim 24, wherein said host cell is a selected from gram-negative bacterium, gram-positive bacterium, chemolithotrophic bacteria, yeast or algae.

63. The method of claim 62, wherein said gram-negative bacterium from the genera Escherichia.

64. The method of claim 62, wherein said gram-positive bacterium from the genera Bacillus.

65. The method of claim 62, wherein said yeast is from the genera Saccharomyces.

66. The method of claim 24, wherein said chemical is 3-hydroxypropionic acid or a derivative of 3-HP, 1,4-butanediol, butanol, isobutanol, polyketide chemical product, or C4-C18 fatty acid chain.

67. The method of claim 24, wherein said chemical is converted to acrylic acid, acrylates, 1,3-propanediol, malonic acid, ethyl-3-hydroxypropionate, ethyl ethoxy propionate, propiolactone, acrylamide, or acrylonitrile to make consumer products.

68. The method of claim 24, wherein said chemical is oligomerized or polymerized to form polyacrylic acid, methyl acrylate, acrylamide, acrylonitrile, propiolactone, ethyl 3-HP, ethyl acrylate, n-butyl acrylate, hydroxypropyl acrylate, hydroxyethyl acrylate, isobutyl acrylate, 2-ethylhexyl acrylate, and acrylic acid ester to which an alkyl or aryl addition may be made, and/or to which halogens, aromatic amines or amides, and aromatic hydrocarbons may be added to make consumer products.

69. A genetically modified organism capable of producing a chemical product of interest, wherein the genetic modification includes introduction of nucleic acid sequences coding for polynucleotides encoding one or more of the following:
(1) an acetyl-CoA carboxylase gene with one or more of its subunits fused together in the genetic structure of the organism;
(2) an acetyl-CoA carboxylase gene having a predefined stoichiometric ratio of each of the four ACCase subunits relative to one another;
(3) a monofunctional malonyl-CoA reductase gene capable of catalyzing the conversion of malonyl-CoA to malonate semialdehyde and one or more genes encoding one or more of the following enzymes: ydfG, mmsB, NDSD, rutE, and nemA;
(4) a monofunctional malonyl-CoA reductase gene capable of catalyzing the conversion of malonyl-CoA to malonate semialdehyde and one or more genes encoding one or more enzymes capable of converting malonate semialdehyde keto form to 3-HP, and one or more genes encoding one or more enzymes capable of converting either the malonate semialdehyde enol form or the malonate semialdehyde hydrated form to 3-HP;
(5) a monofunctional malonyl-CoA reductase enzyme fused to a dehydrogenase enzyme that is either: (a) primarily not NADPH-dependent, (b) primarily NADH-dependent, (c) primarily flavin-dependent, (d) less susceptible to 3-HP inhibition at high concentration, and/or (e) catalyzes a reaction pathway to 3-HP that is substantially irreversible;
(6) a monofunctional malonyl-CoA reductase enzyme fused to one or more dehydrogenase enzymes;
(7) a malonyl-CoA reductase gene that is mutated to enhance its activity at lower temperatures;
(8) salt-tolerant enzymes;
(9) a gene that facilitates the exportation of a chemical product of interest or the export of an inhibitory chemical from within the cell to the extracellular media;
(10) a gene that facilitates the importation from the extracellular media to within the cell of a reactant, precursor, and/or metabolite used in the organism's production pathway for producing a chemical product of interest;
(11) a gene encoding an enzyme in a biosynthetic pathway for converting the carbon source to the chemical product, wherein the gene is encoded into the organism using promoters that are activated by phosphate depletion;
(12) a gene selected from the group consisting of mcr, mmsB, ydfG, rutE, nemA, NDSD, and genes that encode individual or fused subunits of ACCase, wherein the gene is encoded into the organism using a promoter selected from the group consisting of amn, tktB, xasA, yibD, ytfK, pstS, phoH, phnC, and other phosphate-regulated genes;
(13) a gene that is mutated to become activated or deactivated at a given temperature range;
(14) a gene that is mutated such that as a result of a change in the organisms temperature the mutated gene: (1) becomes active and serves a key function in the conversion of the carbon source to the chemical product; or (2) becoming inactive and turns off a branch pathway or other competitive pathway that prevents or limits the pathway leading to the conversion of the carbon source to the chemical product; and
(15) a temperature sensitive mutated gene selected from the group consisting of fabI, fabB, and fabD.
